SEQUENCE LISTING (6 sequences)

SEQ ID NO: 1 - DNA, 16 bp, Artificial Sequence
  SYNTHETIC
tttgggggaaggattc

SEQ ID NO: 2 - DNA, 18 bp, Artificial Sequence
  SYNTHETIC
tcacggaatccttccccc

SEQ ID NO: 3 - protein, 8 aa, Escherichia coli
LEVLFQGP

SEQ ID NO: 4 - DNA, 10 bp, Artificial Sequence
  SYNTHETIC
ttttttttgc

SEQ ID NO: 5 - DNA, 19 bp, Artificial Sequence
  SYNTHETIC
tcaccctcgtacgactctt

SEQ ID NO: 6 - DNA, 29 bp, Artificial Sequence
  SYNTHETIC
gcaaaaaaaaaagagtcgtacgagggtga

(19) United States
(12) Patent Application Publication
(10) Pub. No.: US 2016/0075689 A1
(43) Pub. Date: Mar. 17, 2016

(54) POLYMERASE, ENDONUCLEASE, AND HELICASE INHIBITORS AND METHODS OF USING THEREOF

(71) Applicants: Narsimha Reddy PENTHALA, Little Rock, AR (US); Peter CROOKS, Little Rock, AR (US); Robert EOFF, Little Rock, AR (US); Grace COGGINS, Little Rock, AR (US); Leena MADDUKRI, Little Rock, AR (US); Jessica H. HARTMAN, Little Rock, AR (US); Dae Song JANG, Little Rock, AR (US); Alexei BASNAKIAN, Little Rock, AR (US); Suja AARATTUTHODIYIL, Little Rock, AR (US); Kevin RANEY, Little Rock, AR (US); THE BOARD OF TRUSTEES OF THE UNIVERSITY OF ARKANSAS, Little Rock, AR (US)

(72) Inventors: Narsimha Reddy Penthala, Little Rock, AR (US); Peter Crooks, Little Rock, AR (US); Robert Eoff, Little Rock, AR (US); Grace Coggins, Little Rock, AR (US); Leena Maddukuri, Little Rock, AR (US); Jessica H. Hartman, Little Rock, AR (US); Dae Song Jang, Little Rock, AR (US); Alexei Basnakian, Little Rock, AR (US); Suja Aarattuthodiyil, Little Rock, AR (US); Kevin Raney, Little Rock, AR (US)

(73) Assignee: The Board of Trustees of the University of Arkansas, Little Rock, AR (US)

(21) Appl. No.: 14/786,331

(22) PCT Filed: Apr. 23, 2014
(86) PCT No.: PCT/US14/35169

§ 371 Date: Oct. 22, 2015

Related U.S. Application Data
(60) Provisional application No. 61/815,063, filed on Apr. 23, 2013.
(60) Provisional application No. 61/868,879, filed on Aug. 22, 2013.
(60) Provisional application No. 61/901,715, filed on Nov. 8, 2013.
(60) Provisional application No. 61/901,708, filed on Nov. 8, 2013.

(51) Int. Cl. C07D 403/06

(52) U.S. Cl. CPC C07D 403/06

(57) ABSTRACT

Inhibitors of DNA damage polymerases, endonucleases, and helicases are provided. In particular, compounds comprising Formula (I) are described.

### CROSS-REFERENCE TO RELATED APPLICATIONS

This application claims the benefit of PCT Application PCT/US2014/035169, filed Apr. 23, 2014, which claims the benefit of U.S. Provisional Patent Application No. 61/815,063, filed Apr. 23, 2013, U.S. Provisional Patent Application No. 61/868,879, filed Aug. 22, 2013, U.S. Provisional Application No. 61/901,715, filed Nov. 8, 2013 and U.S. Provisional Patent Application No. 61/901,708 filed Nov. 8, 2013, each of the disclosures of which are hereby incorporated by reference in their entirety.

### GOVERNMENTAL RIGHTS

This invention was made with government support under Federal Grant GM103429 awarded by the National Institute of General Medical Sciences and by Federal Grant R00 GM084460 awarded by the National Institutes of Health. The government has certain rights in the invention.

### FIELD OF THE INVENTION

The invention describes novel compounds with activity as polymerase inhibitors, endonuclease inhibitors, and helicase inhibitors.

### BACKGROUND OF THE INVENTION

The invention relates to novel N-alkyl and N-aroyl-1H-indol-3-yl methylene-barbituates or 2-thiobarbituates that are biologically active and may be useful in a variety of contexts.

The process of replicating deoxynucleic acids (DNA) in a timely manner is perturbed by exogenous and endogenous factors, including DNA adducts and natural replication fork barriers, such as G-quadruplex forming sequences. Perturbed DNA replication induces replication stress response (RSR) mechanisms that recruit specialized DNA polymerases to sites of replication stress. These so-called RSR polymerases assist replication fork progression by moving error-prone DNA synthesis past the offending lesion instead of performing repair. RSR polymerases are up-regulated in some cancers, contributing to the progression of the disease by promoting increased genomic instability. In addition, cancer therapies that act to limit tumor growth through the induction of DNA damage in cancer cells are often rendered ineffective through the stimulation of DNA repair mechanisms, which results in resistance of cancers to the damaging effects of the compound.

Further, DNA fragmentation is a limiting and necessary mechanism of cell death and is catalyzed by a group of enzymes called “apoptotic endonucleases.” One of the most active representatives of this group is Endonuclease G (EndoG), a nuclear DNA-coded mitochondrial enzyme that relocates to the nucleus and fragments DNA during apoptosis. Currently, there are no pharmaceutically viable chemical inhibitors of EndoG. Such inhibitors would be useful for protection of normal tissues from various injuries, including irradiation, chemical/drug poisoning, hypoxia, or physical injury. Inhibitors of EndoG endonuclease would also be useful for increasing resistance of normal tissues surrounding tumors when DNA-damaging and cell death-inducing chemotherapeutics are used to promote cell death in cancer cells.

Finally, around 170 million people worldwide are infected with HCV that may lead to liver cirrhosis and hepatocellular carcinoma and is the major reason for liver transplantation. No vaccine is currently available. The standard treatment, consisting of a combination of interferon alpha with ribavirin plus a protease inhibitor such as telaprevir is effective but is extremely expensive and causes severe side effects. Telaprevir and boceprevir (NS3-4A protease inhibitors), were recently approved for the treatment of chronic hepatitis C patients. The triple combination therapy with interferon, ribavirin, and telaprevir exhibits side effects including hemolytic anemia, fatigue, flu-like symptoms, birth defects, and depression. Furthermore, the emergence of drug-resistant viruses is a serious problem with therapies that use antiviral compounds. For these reasons, there is an urgent need to develop more effective and better tolerated treatments.

The development of antiviral agents directly targeting the viral life cycle seems to be the most promising therapeutic strategy, as it should block HCV replication and, thus, the spread of infection. This goal could be achieved by direct inhibition of viral enzymes involved in the replication process. Since microbes synthesize their NA genomes in a template dependent manner, in addition to DNA or RNA polymerases, a helicase is required replication. Helicases unwind duplex NA structures and are promising antiviral drug targets because their enzymatic activities are essential for viral genome replication, transcription, and translation. NS3 helicase is an enzyme indispensable for HCV replication and appears to be an attractive target for development of HCV-specific antiviral therapies. Another advantage is that NS3 helicase does not possess close homologues among human cancer cellular enzymes. Its inhibitors could be used together with inhibitors of other viral proteins in a cocktail, preventing HCV from escaping the treatment by the emergence of drug-resistant mutants. Inhibition of helicase activity could be achieved by inhibiting binding of the enzyme to the NA substrate, NTP binding and hydrolysis, and NTP-hydrolysis-dependent unwinding of the duplex substrate.

There remains a need for compounds for the inhibition of polymerase, endonuclease, and helicase.

### BRIEF DESCRIPTION OF THE FIGURES

The application file contains at least one photograph executed in color. Copies of this patent application publication with color photographs will be provided by the Office upon request and payment of the necessary fee.

FIG. 1A and FIG. 1B depict a diagram and a plot showing the assay used to screen for polymerase inhibitors. (FIG. 1A) Polymerase activity separates a short TAMRA-labeled oligonucleotide from its BHQ2-labeled complement. Fluorescence emission at λₑₘ=598 nm is monitored over time. (FIG. 1B) Mean (±standard deviation) of hpol η¹⁻⁴³⁷ activity plotted as a function of time. The inset shows the slope of the initial portion of the velocity curve that was used to estimate the rate of polymerase-catalyzed strand displacement: v₀=10.2±0.4 nM min⁻¹.

FIG. 2A, FIG. 2B, FIG. 2C and FIG. 2D graphically depict the identification of potential small-molecule inhibitors of hpol η. Hpol η activity was measured in the presence of 320 novel compounds. Four 96-well plates (shown in panels (FIG. 2A)-(FIG. 2D) containing the 320 compounds were tested. The rate of product formation in the presence of the compounds was normalized against the rate of product formation for the DMSO control experiment. The experiments were repeated in triplicate and the mean (±standard deviation) is shown. Potential inhibitors were identified as compounds that inhibited polymerase activity more than one standard deviation from the mean of the control experiment (red bars).

FIG. 3A, FIG. 3B and FIG. 3C graphically depict the determination of IC₅₀ for ITBA-3 mediated inhibition of hpol q activity. (FIG. 3A) The chemical structure of ITBA-3. (FIG. 3B) Hpol η (10 nM) activity was monitored using the fluorescence-based assay in the presence of increasing amounts of ITBA-3: DMSO control (black), 1 μM (blue), 5 μM (cyan), 10 μM (green), 25 μM (orange), 50 μM (red), 100 μM (magenta) and 250 μM (purple). (FIG. 3C) Hpol η activity was plotted as a function of the log of inhibitor concentration and fit to equation 1 described in the materials and methods section of the Examples to determine the IC₅₀ value. The mean (±standard deviation) of the three data sets is shown.

FIG. 4 graphically depicts ITBA-3 specificity for inhibition of hpol η. The IC₅₀ values for inhibition of different polymerases by ITBA-3 are shown. The mean (±standard deviation) of the three data sets is shown.

FIG. 5A and FIG. 5B depict structure activity relationships for ITBA derivatives and inhibition of hpol η. (FIG. 5A) Chemical structure of the ITBA scaffold. (FIG. 5B) Graph showing the structure-activity relationships for inhibition of hpol n activity by ITBA derivatives described in Table 4. Hpol η activity was measured in the presence of either DMSO or 50 μM of the indicated ITBA derivative.

FIG. 6A and FIG. 6B depict the determination of the IC₅₀ value for ITBA-17 mediated inhibition of hpol η. (FIG. 6A) Chemical structure of ITBA-17. (FIG. 6B) Hpol η activity was measured in the presence of increasing amounts of ITBA-17 to be roughly half that of ITBA-3.

FIG. 7A, FIG. 7B and FIG. 7C depict the validation of ITBA-12 as an inhibitor of hpol η activity. (FIG. 7A) Hpol q-catalyzed displacement of the TAMRA-labeled oligonucleotide from the BHQ2-labeled template was monitored over time in the presence of DMSO (black circles), 10 μM (yellow squares), 20 μM (orange triangles) and 60 μM (red inverted triangles) ITBA-12. (FIG. 7B) The rate of product formation for the reactions shown in panel A was determined by linear regression and is plotted for each reaction. Increasing amounts of ITBA-12 produced a pronounced decrease in the rate of product formation. (FIG. 7C) Hpol η (2 nM) catalyzed extension of the FAM-16mer/18-mer primer-template DNA (200 nM) was allowed to proceed in the presence of a mixture of all four dNTPs (1 mM each) and MgCl₂ (10 mM). The products were separated by using denaturing PAGE (16% polyacrylamide/7 M urea). Inhibition of polymerase activity is most clear at 60 μM ITBA-12.

FIG. 8A and FIG. 8B depict two graphs showing anti-EndoG activity of compounds from chemical library with compounds used at a concentration of (FIG. 8A) 0.1 μM, and at a concentration of (FIG. 8B) 1 μM.

FIG. 9 depicts an agarose gel used for testing the potential EndoG inhibitors from the primary screen in a plasmid incision assay. The substrate, supercoiled plasmid DNA in 1% DMSO (lane 1) and water (lane 4), is converted by EndoG into open circular and linear DNAs (lane 2). Conversion of supercoiled plasmids to open circle plasmid by EndoG is inhibited in the presence of a positive control inhibitor ZnCl₂ (lane 3) as well as selected inhibitors PNR-3-80 (lane 7) and PNR-3-82 (lane 8).

FIG. 10A and FIG. 10B graphically depict the determination of IC₅₀ of compounds PNR-3-80 and PNR-3-82. (FIG. 10A) A plot of EndoG activity as a function of PNR-3-80 compound concentration. (FIG. 10B) A plot of EndoG activity as a function of PNR-3-82 compound concentration.

FIG. 11A and FIG. 11B graphically depict specificity of compounds PNR-3-80 and PNR-3-82 against EndoG compared to activity against DNase I. (FIG. 11A) A plot of EndoG (red curve) activity and DNase I activity (blue curve) as a function of PNR-3-80 compound concentration. (FIG. 11B) A plot of EndoG (red curve) activity and DNase I activity (blue curve) as a function of PNR-3-82 compound concentration.

FIG. 12A and FIG. 12B graphically depict modulation of alternative splicing of nucleic acid sequences encoding DNase I by inhibitors of EndoG. Relative levels of nucleic acid sequences encoding EndoG (grey bars), the full length DNase I isoform (red bars), and the Δ4DNase I isoform (blue bars) in ZR-75-1 cells treated with PNR-3-80 (FIG. 12A), and PNR-3-82 (FIG. 12B).

FIG. 13A and FIG. 13B graphically depict screening of the helicase inhibitors using the fluorescence based helicase assay. (FIG. 13A) Helicase catalyzes the unwinding of FAM-labeled DNA from its compliment. The resulting increase in fluorescence is monitored over time and plotted using Graph Pad Prisim software. The slope of the initial part of the plot was used to calculate helicase activity. (FIG. 13B) NS3 helicase activity in the presence of the compounds (20 μM) was plotted. The data represent the average of three separate experiments with standard deviations.

FIG. 14A, FIG. 14B and FIG. 14C show analysis of ITBA-3-79, ITBA-3-82, and ITBA-3-85 mediated inhibition of NS3 helicase. (FIG. 14A) The chemical structures of the compounds are shown, (FIG. 14B) Helicase activity was quantitated using a gel-based assay in the presence of ITBA-3-79, ITBA-3-82, and ITBA-3-85. (FIG. 14C) Determination of the IC₅₀ mediated inhibition of NS3 helicase activity for ITBA-3-79, ITBA-3-82, and ITBA-3-85. Helicase activity was plotted as a function of the log of inhibitor concentration to determine the IC₅₀ value. The mean±standard deviation of three data sets is shown.

FIG. 15A and FIG. 15B show determination of the ATPase and protease activities in the presence of ITBA-3-79, ITBA-3-82 and ITBA-3-85. (FIG. 15A) The ATPase activity of NS3 (50 nM) was analyzed using a coupled spectrophotometric assay. (FIG. 15B) For the protease assay, 50 nM NS3-4A and 100 nM substrate (Ac-Asp-Glu-Asp-EDANS-Glu-Glu-Abu-L-Lactoyl-Ser-Lys DABCYL-NH2) was used. The emission spectra of EDANS and the absorption spectra of DABCYL overlap making the peptide internally quenched. Cleaving of the substrate by the protease results in an increase in fluorescence that can be measured (λₑₓ−355 nm; λₑₘ−500 nm).

FIG. 16A shows NS3 helicase activity in the presence of the compounds (25 μM) was plotted. The mean±standard deviation of three data sets is shown. (FIG. 16B) Native PAGE images of the unwinding of 2 μM 15T22 bp DNA by 100 nM NS3 in the presence of ITBA-3-79, ITBA-3-82 or ITBA-3-85 (25 μM) for the time indicated. The unwinding reaction was stopped by the addition of 400 mM EDTA. The ssDNA product forms over time and is separated from dsDNA substrate by 20% native PAGE. Radioactivity was visualized using a Phosphor Imager.

FIG. 17A, FIG. 17B, FIG. 17C and FIG. 17D show the effects of PNR-3-80 and PNR-3-82 on (FIG. 17A) RNase; (FIG. 17B) Protease; (FIG. 17C) LDH, and (FIG. 17D) SOD. No inhibiting activity was found for these non-nuclease enzymes.

FIG. 18 shows the effect of exposing cisplatin (60 μM) to 22Rv1 cells, which naturally express EndoG in the presence or absence of P-NR-30. P-NR-30 showed complete inhibition of Cisplatin-induced cell death compared to control (without inhibitor).

FIG. 19A and FIG. 19B show the results of tests to confirm cytoprotective properties of the compounds. PC3 cells were transferred with EndoG gene bound to cyan fluorescent protein (CFP). (FIG. 19A) shows the blue/cyan fluorescence of the resulting EndoG-expressing cells. (FIG. 19B) shows the results of exposing intact PC3 and EndoG-expressing PC3 cells with Docetaxel (80 μM) in the presence or absence of inhibitors PNR-3-80 and PNR-3-82 (50 μM) each. Cell death was measured by TUNEL assay.

### DETAILED DESCRIPTION OF THE INVENTION

Compounds capable of inhibiting DNA repair polymerase, endonucleases, and helicases have been discovered. The compounds may advantageously be useful for treating or preventing disease. The invention also encompasses a process for preparing and using a compound of the invention.

### I. Compounds

Compounds of the invention generally comprise Formula (I):

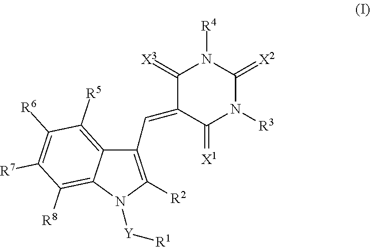

wherein:
X¹, X², and X³ are each independently selected from the group consisting of oxygen, sulfur, and sulfene;
Y is selected from the group consisting of CH₂, carbonyl, sulfide, sulfone, and sulfoxide;
R¹ is selected from the group consisting of hydrocarbyl, substituted hydrocarbyl, cyano and COOCH₃;
R², R³, and R⁴ are each independently selected from the group consisting of hydrogen, hydrocarbyl, and substituted hydrocarbyl; and
R⁵, R⁶, R⁷, and R⁸ are each independently selected from the group consisting of hydrogen, hydrocarbyl, substituted hydrocarbyl, trifluoromethyl, halogen, cyano, nitro, amidine, amino, carboxyl, ester, alkylalkylamino, dialkylamino, hydroxyl, alkoxy, and arylalkoxy.

In some embodiments for compounds comprising Formula (I), X¹ and X³ are together selected from the group consisting of oxygen, sulfur, and sulfene. In some alternatives of the embodiments, X¹ and X³ are sulfur. In other alternatives of the embodiments, X¹ and X³ are sulfene. In exemplary alternatives of the embodiments, X¹ and X³ are oxygen.

In some embodiments for compounds comprising Formula (I), R⁵ and R⁸ may together be selected from the group consisting of hydrogen, hydrocarbyl, substituted hydrocarbyl, halogen, cyano, nitro, amidine, amino, carboxyl, ester, alkylalkylamino, dialkylamino, hydroxyl, alkoxy, and arylalkoxy. In some exemplary alternatives of the embodiments, R⁵ and R⁸ are hydrogen.

For each of the foregoing embodiments for compounds comprising Formula (I), R² may be selected from the group consisting of hydrogen, methyl, phenyl, and substituted phenyl. In some alternatives of the embodiment, R² is methyl. In other alternatives of the embodiment, R² is phenyl. In yet other alternatives of the embodiment, R² is substituted phenyl. In preferred alternatives of the embodiment, R² is hydrogen.

In each of the foregoing embodiments for compounds comprising Formula (I), R³ and R⁴ may together be selected from the group consisting of hydrogen, hydrocarbyl, and substituted hydrocarbyl. In some exemplary alternatives of the embodiments, R³ and R⁴ are hydrogen.

For each of the foregoing embodiments for compounds comprising Formula (I), X² is oxygen. In other embodiments, X² is sulfene. In preferred embodiments, X² is sulfur.

For each of the foregoing embodiments for compounds comprising Formula (I), R¹ may be selected from the group consisting of phenyl, substituted phenyl, biphenyl, substituted biphenyl, naphthyl, substituted naphthyl, and cyano.

In some embodiments, compounds of the invention comprise a compound of Formula (II):

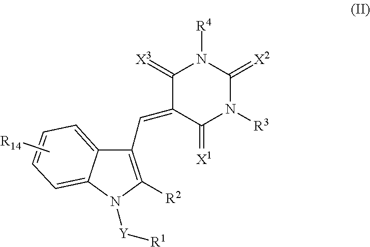

wherein:
X¹, X², and X³ are each independently selected from the group consisting of oxygen, sulfur, and sulfene;
Y is selected from the group consisting of CH₂, carbonyl, sulfide, sulfone, and sulfoxide;
R¹ is selected from the group consisting of hydrocarbyl, substituted hydrocarbyl, cyano, and COOCH₃;
R², R³, and R⁴ are each independently selected from the group consisting of hydrogen, hydrocarbyl, and substituted hydrocarbyl; and
R¹⁴ is selected from the group consisting of hydrogen, halogen, trifluoromethyl, methoxy and COOCH₃.

TABLE 1

| R¹⁴ | R¹ | X¹ and X³ | X² |
|---|---|---|---|
| hydrogen | C₆H₅ | sulfur or oxygen | sulfur or oxygen |
| chlorine | C₆H₅ | sulfur or oxygen | sulfur or oxygen |
| bromine | C₆H₅ | sulfur or oxygen | sulfur or oxygen |
| OCH₃ | C₆H₅ | sulfur or oxygen | sulfur or oxygen |
| hydrogen | 4-F—C₆H₄ | sulfur or oxygen | sulfur or oxygen |
| chlorine | 4-F—C₆H₄ | sulfur or oxygen | sulfur or oxygen |
| bromine | 4-F—C₆H₄ | sulfur or oxygen | sulfur or oxygen |
| OCH₃ | 4-F—C₆H₄ | sulfur or oxygen | sulfur or oxygen |
| hydrogen | 4-OCH₃—C₆H₄ | sulfur or oxygen | sulfur or oxygen |
| chlorine | 4-OCH₃—C₆H₄ | sulfur or oxygen | sulfur or oxygen |
| bromine | 4-OCH₃—C₆H₄ | sulfur or oxygen | sulfur or oxygen |
| OCH₃ | 4-OCH₃—C₆H₄ | sulfur or oxygen | sulfur or oxygen |
| hydrogen | 4-CN—C₆H₄ | sulfur or oxygen | sulfur or oxygen |
| chlorine | 4-CN—C₆H₄ | sulfur or oxygen | sulfur or oxygen |
| bromine | 4-CN—C₆H₄ | sulfur or oxygen | sulfur or oxygen |
| OCH₃ | 4-CN—C₆H₄ | sulfur or oxygen | sulfur or oxygen |
| hydrogen | 4-COOCH₃—C₆H₄ | sulfur or oxygen | sulfur or oxygen |
| chlorine | 4-COOCH₃—C₆H₄ | sulfur or oxygen | sulfur or oxygen |
| bromine | 4-COOCH₃—C₆H₄ | sulfur or oxygen | sulfur or oxygen |
| OCH₃ | 4-COOCH₃—C₆H₄ | sulfur or oxygen | sulfur or oxygen |
| hydrogen | 2-Br—C₆H₄ | sulfur or oxygen | sulfur or oxygen |
| chlorine | 2-Br—C₆H₄ | sulfur or oxygen | sulfur or oxygen |
| bromine | 2-Br—C₆H₄ | sulfur or oxygen | sulfur or oxygen |
| OCH₃ | 2-Br—C₆H₄ | sulfur or oxygen | sulfur or oxygen |
| hydrogen | 1-naphthyl | sulfur or oxygen | sulfur or oxygen |
| chlorine | 1-naphthyl | sulfur or oxygen | sulfur or oxygen |
| bromine | 1-naphthyl | sulfur or oxygen | sulfur or oxygen |
| OCH₃ | 1-naphthyl | sulfur or oxygen | sulfur or oxygen |
| hydrogen | 2-naphthyl | sulfur or oxygen | sulfur or oxygen |
| chlorine | 2-naphthyl | sulfur or oxygen | sulfur or oxygen |
| bromine | 2-naphthyl | sulfur or oxygen | sulfur or oxygen |
| OCH₃ | 2-naphthyl | sulfur or oxygen | sulfur or oxygen |
| hydrogen | CN | sulfur or oxygen | sulfur or oxygen |
| chlorine | CN | sulfur or oxygen | sulfur or oxygen |
| bromine | CN | sulfur or oxygen | sulfur or oxygen |
| OCH₃ | CN | sulfur or oxygen | sulfur or oxygen |
| hydrogen | COOCH₃ | sulfur or oxygen | sulfur or oxygen |
| chlorine | COOCH₃ | sulfur or oxygen | sulfur or oxygen |
| bromine | COOCH₃ | sulfur or oxygen | sulfur or oxygen |
| OCH₃ | COOCH₃ | sulfur or oxygen | sulfur or oxygen |

In some embodiments for compounds comprising Formula (II):
X¹ and X³ are oxygen;
Y is carbonyl;
X² is sulfur;
R² is hydrogen;
R¹ is selected from the group consisting of phenyl, 2-bromophenyl, 4-fluorophenyl, 4-methoxy-phenyl, 4-COOCH₃-phenyl, 2-naphtyl, 1-naphthyl, cyano, and COOCH₃; and
R¹⁴ is selected from the group consisting of chlorine, bromine, and methoxy.

In some embodiments, compounds of the invention comprise a compound of Formula (III):

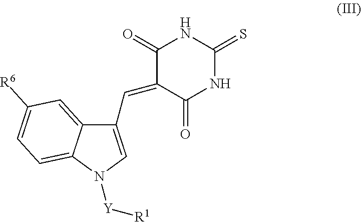

wherein:
R¹ is selected from the group consisting of hydrocarbyl, substituted hydrocarbyl, and cyano; and
R⁶ is selected from the group consisting of hydrogen, hydrocarbyl, substituted hydrocarbyl, halogen, cyano, nitro, amidine, amino, carboxyl, ester, alkylalkylamino, dialkylamino, hydroxyl, alkoxy, and arylalkoxy.

In some embodiments for compounds comprising Formula (III):
R⁶ is selected from the group of hydrogen, halogen and methoxy;
Y is carbonyl; and
R¹ is selected from the group consisting of phenyl, 2-bromophenyl, 4-fluorophenyl, 4-methoxy-phenyl, 2-naphtyl, 4-COOCH₃-phenyl, 1-naphthyl, cyano, and COOCH₃.

In some embodiments, compounds of the invention comprise the compounds of Formula (IV):

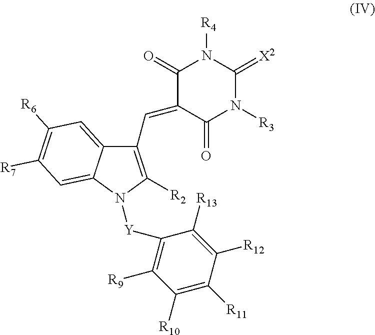

wherein:
X² is selected from the group consisting of oxygen, sulfur, and sulfene;
Y is selected from the group consisting of CH₂, carbonyl, sulfide, sulfone, and sulfoxide;
R², R³, and R⁴ are each independently selected from the group consisting of hydrogen, hydrocarbyl, and substituted hydrocarbyl;
R⁶ and R⁷ are each independently selected from the group consisting of hydrogen, hydrocarbyl, substituted hydrocarbyl, halogen, cyano, nitro, amidine, amino, carboxyl, ester, alkylalkylamino, dialkylamino, hydroxyl, alkoxy, and arylalkoxy; and
R⁹, R¹⁰, R¹¹, R¹², and R¹³ are each independently selected from the group consisting of hydrogen, hydrocarbyl, substituted hydrocarbyl, trifluoromethyl, halogen, cyano, nitro, amidine, amino, carboxyl, ester, alkylalkylamino, dialkylamino, hydroxyl, alkoxy, and arylalkoxy.

In some embodiments for compounds comprising Formula (IV):
Y is carbonyl;
X² is sulfur; and
R², R³ and R⁴ are hydrogen;
R⁶ and R⁷ are each independently selected from the group consisting of hydrogen, hydrocarbyl, substituted hydrocarbyl, halogen, cyano, nitro, amidine, amino, carboxyl, ester, alkylalkylamino, dialkylamino, hydroxyl, alkoxy, and arylalkoxy; and
R⁹, R¹⁰, R¹¹, R¹², and R¹³ are each independently selected from the group consisting of hydrogen, hydrocarbyl, substituted hydrocarbyl, trifluoromethyl, halogen, cyano, nitro, amidine, amino, carboxyl, ester, alkylalkylamino, dialkylamino, hydroxyl, alkoxy, and arylalkoxy.

In other embodiments, compounds of the invention comprise a compound of Formula (V):

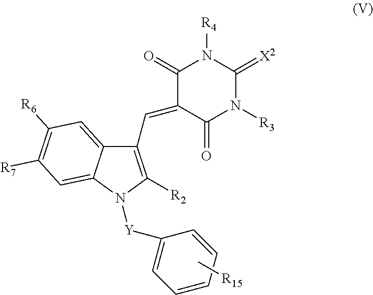

wherein:
X² is selected from the group consisting of oxygen, sulfur, and sulfene;
Y is selected from the group consisting of CH₂, carbonyl, sulfide, sulfone, and sulfoxide;
R², R³, and R⁴ are each independently selected from the group consisting of hydrogen, hydrocarbyl, and substituted hydrocarbyl;
R⁶ and R⁷ are each independently selected from the group consisting of hydrogen, hydrocarbyl, substituted hydrocarbyl, halogen, cyano, nitro, amidine, amino, carboxyl, ester, alkylalkylamino, dialkylamino, hydroxyl, alkoxy, and arylalkoxy; and
R¹⁵ is selected from the group consisting of hydrogen, hydrocarbyl, substituted hydrocarbyl, trifluoromethyl, halogen, cyano, nitro, amidine, amino, carboxyl, ester, alkylalkylamino, dialkylamino, hydroxyl, alkoxy, and arylalkoxy.

In some embodiments for compounds comprising Formula (V):
Y is carbonyl;
X² is sulfur;
R², R³ and R⁴ are hydrogen;
R⁶ and R⁷ are each independently selected from the group consisting of hydrogen, halogen, and methoxy; and
R¹⁵ is selected from the group of hydrogen, halogen, cyano, methoxy, and COOCH₃.

In yet other embodiments, compounds of the invention comprise a compound of Formula (VI):

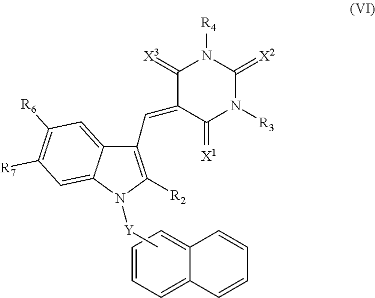

wherein:
X¹, X², and X³ are each independently selected from the group consisting of oxygen, sulfur, and sulfene;
Y is selected from the group consisting of CH₂, carbonyl, sulfide, sulfone, and sulfoxide;
R², R³, and R⁴ are each independently selected from the group consisting of hydrogen, hydrocarbyl, and substituted hydrocarbyl; and
R⁶ and R⁷ are each independently selected from the group consisting of hydrogen, hydrocarbyl, substituted hydrocarbyl, halogen, cyano, nitro, amidine, amino, carboxyl, ester, alkylalkylamino, dialkylamino, hydroxyl, alkoxy, and arylalkoxy.

In some embodiments for compounds comprising Formula (VI):
X¹ and X³ are oxygen;
Y is carbonyl;
X² is sulfur; and
R², R³and R⁴ are hydrogen; and
R⁶ and R⁷ are each independently selected from the group consisting of hydrogen, halogen, and methoxy.

In yet other embodiments, compounds of the invention comprise a compound of Formula (VII):

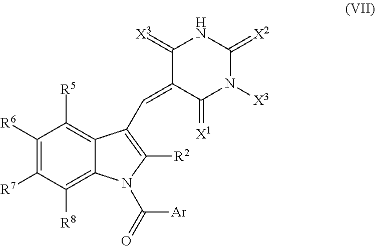

wherein:
Ar is substituted or unsubstituted aryl;
X¹, X², and X³ are each independently selected from the group consisting of oxygen, sulfur, and sulfene;
R², R³, and R⁴ are each independently selected from the group consisting of hydrogen, hydrocarbyl, and substituted hydrocarbyl; and
R⁵, R^{6′} R⁷, and R⁸ are each independently selected from the group consisting of hydrogen, hydrocarbyl, substituted hydrocarbyl, halogen, cyano, nitro, amidine, amino, carboxyl, ester, alkylalkylamino, dialkylamino, hydroxyl, alkoxy, and arylalkoxy.

In some embodiments for compounds comprising Formula (VII):
X¹ and X³ are oxygen;
X² is sulfur; and
R², R³and R⁴ are hydrogen; and
R^{6′} R⁷, and Ware each independently selected from the group consisting of hydrogen, halogen, and methoxy.

In exemplary embodiments, compounds of the invention comprise a compound selected from the group of compounds in Table 2.

TABLE 2

| | |
|---|---|
| (I)(a) (ITBA-3) | 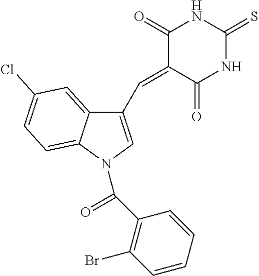 [SMILES: O=C1NC(=S)NC(=O)C1=Cc1cn(C(=O)c2ccccc2Br)c2ccc(Cl)cc12] |
| (I)(b) | 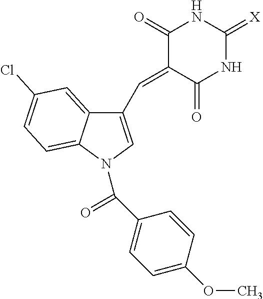 |
| (I)(c) | 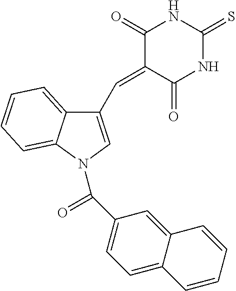 [SMILES: O=C1NC(=S)NC(=O)C1=Cc1cn(C(=O)c2ccc3ccccc3c2)c2ccccc12] |
| (I)(d) (ITBA-17) | 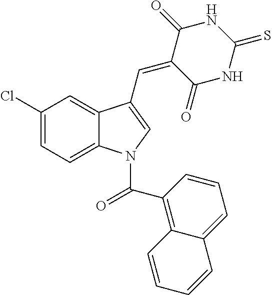 [SMILES: O=C1NC(=S)NC(=O)C1=Cc1cn(C(=O)c2cccc3ccccc23)c2ccc(Cl)cc12] |
| (I)(e) (PNR-3-80) | 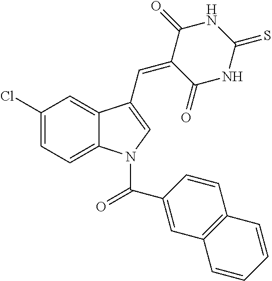 [SMILES: O=C1NC(=S)NC(=O)C1=Cc1cn(C(=O)c2ccc3ccccc3c2)c2ccc(Cl)cc12] |
| (I)(f) | 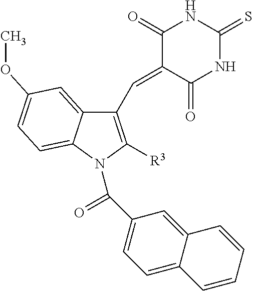 |
| (I)(g) ITBA-3-79 | 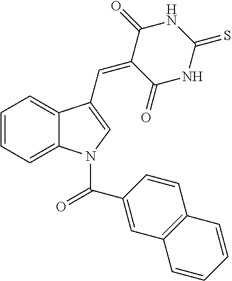 [SMILES: O=C1NC(=S)NC(=O)C1=Cc1cn(C(=O)c2ccc3ccccc3c2)c2ccccc12] |
| (I)(h) (PNR-3-82) (ITBA-3-82) | 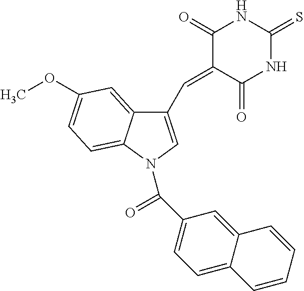 [SMILES: COc1ccc2c(c1)c(C=C1C(=O)NC(=S)NC1=O)cn2C(=O)c1ccc2ccccc2c1] |
| (I)(i) ITBA-3-85 | 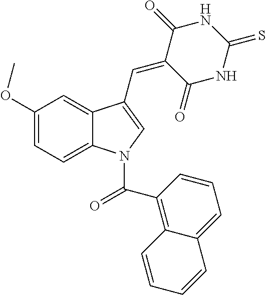 [SMILES: COc1ccc2c(c1)c(C=C1C(=O)NC(=S)NC1=O)cn2C(=O)c1cccc2ccccc12] |

### (a) DNA Repair

Compounds of the invention are capable of inhibiting DNA repair polymerase enzymes. As will be recognized by those skilled in the art, DNA repair polymerase enzymes may be any polymerase enzyme that may be used in a process by which a cell identifies and corrects DNA damage that may be caused by metabolic factors such as reactive oxygen species, or environmental factors such as ultraviolet and other radiation frequencies, toxins, mutagenic chemicals, viruses, and DNA damaging chemotherapeutic agents. DNA repair polymerase enzymes may, for instance, be required for short-patch base excision repair essential for repairing alkylated bases, oxidized bases, or abasic sites, non-homologous end-joining essential for rejoining DNA double-strand breaks, and DNA repair by translesion synthesis. In some embodiments, compounds of the invention inhibit a polymerase enzyme required for short-patch base excision repair. In other embodiments, compounds of the invention inhibit a polymerase enzyme required for non-homologous end-joining. In yet other embodiments, compounds of the invention inhibit a polymerase enzyme required for translesion synthesis.

Non-limiting examples of DNA repair polymerases include family X polymerases such as polymerase sigma (pol σ), polymerase beta (pol β), polymerase lambda (pol λ), and polymerase mu (pol μ), and family Y polymerases such as polymerase eta (pol η), polymerase iota (pol ι), and polymerase kappa (pol κ). In some embodiments, compounds of the invention inhibit pol σ. In other embodiments, compounds of the invention inhibit pol μ. In yet other embodiments, compounds of the invention inhibit pol ι. In other embodiments, compounds of the invention inhibit pol κ. In some preferred embodiments, compounds of the invention inhibit pol β. In exemplary embodiments, compounds of the invention inhibit pol η.

Compounds of the invention may be capable of inhibiting one or more than one DNA repair polymerase. For instance, a compound may be capable of inhibiting 1, 2, 3, 4, 5, or more DNA repair polymerases. Preferably, a compound is capable of inhibiting 1, 2, or 3 DNA repair polymerases. In exemplary embodiments, a compound of the invention is capable of inhibiting pol β and pol η.

Any method of measuring polymerase activity may be used to measure inhibition of polymerase activity by compounds of the invention. Non limiting examples of polymerase activity assays that may be used to measure inhibition of polymerase activity by compounds of the invention may include polymerase-catalyzed displacement of labeled oligonucleotide and primer extension assays, and may be as described in the examples and in, e.g., Yamanaka et al., 2012, PLoS One 7:e45032, and Dorjsuren et al., 2009, Nucleic Acids Res. 37:e128, the disclosures of which are incorporated herein in their entirety.

In general, titration curves measuring the ability of a compound to inhibit polymerase activity may be performed to determine the IC₅₀. In some embodiments, the IC₅₀ of a compound may be less than about 100, 90, 80, 70, 60, 50, 40, 30, 20, or about 10 μM. In other embodiments, the IC₅₀ of a compound may be less than about 50, 45, 40, 35, 30, 25, 20, 15, 10, 5, or about 1 μM. In yet other embodiments, the IC₅₀ of a compound may be less than about 30, 29, 28, 27, 26, 25, 24, 23, 22, 21, 20, 19, 18, 17, 16, 15, 14, 13, 12, 11, 10, 9, 8, 7, 6, 5, 4, 3, 2, or about 1 μM. In preferred embodiments, the IC₅₀ of a compound may be less than about 30 μM. In other preferred embodiments, the IC₅₀ of a compound may be about 14, 15, 16, or about 17 μM.

Alternatively, percent activity of a compound may be determined by measuring polymerase activity in the presence of the compound, and comparing the polymerase activity to a control polymerase activity as determined in the absence of the compound. In some embodiments, percent activity of a 50 μM concentration of a compound may be less than about 40, 30, 20, 10, or about 5%. In preferred embodiments, percent activity of a 50 μM concentration of a compound may be less than about 25, 20, 15, 10, 5, or about 1%.

Activity of a compound of the invention may also be determined by determining modulation by the compound of survival of a cell contacted with a DNA damaging chemotherapeutic agent. As described in Section IV(a) below, a tumor cell expressing a DNA damaging polymerase may be resistant to a DNA damaging chemotherapeutic. As such, contacting such a tumor cell with a compound of the invention may attenuate the resistance of the tumor cell to the DNA damaging chemotherapeutic.

While not wishing to be bound by theory, it is believed that compounds of the invention may inhibit polymerase activity by inhibiting binding of nucleotide substrate to the polymerase.

### (b) Endonuclease Inhibition

Compounds of the invention are capable of inhibiting endonuclease enzymes. As will be recognized by those skilled in the art, endonuclease enzymes are enzymes that cleave the phosphodiester bond within a polynucleotide chain. In general, compounds of the invention are capable of inhibiting endonucleases normally active during apoptosis. Non-limiting examples of apoptotic endonucleases include endonuclease G (EndoG) and deoxyribonuclease I (DNase I). In preferred embodiments, compounds of the invention inhibit EndoG activity.

Any method of measuring endonuclease activity may be used to measure inhibition of endonuclease activity by compounds of the invention. In general, methods of measuring endonuclease activity include any method that may be used to measure cleavage of a phosphodiester bond within a polynucleotide chain. As will be recognized by those of skill in the art, methods of measuring endonuclease activity can and will vary depending on the type of endonuclease, and whether the activity is measured in vitro, in vivo, or ex vivo. Non-limiting examples of endonuclease activity assays that may be used to measure inhibition of an apoptotic endonuclease by compounds of the invention may include labeled nucleic acid probes, plasmid incision assays, assays based on DNA fragmentation, or assays based on nucleic acid amplification.

In some embodiments, endonuclease activity is measured using a plasmid incision assay. In a preferred embodiment, endonuclease activity is measured using a plasmid incision assay as described in the Examples. In other embodiments, endonuclease activity is measured using labeled nucleic acid probes. In a preferred embodiment, endonuclease activity is measured using labeled nucleic acid probes as described in the examples and in US provisional patent filed Oct. 19, 2012, Ser. No. 61/716,097, the disclosure of which is incorporated herein in its entirety.

In general, titration curves measuring the ability of a compound to inhibit endonuclease activity may be performed to determine the IC₅₀. In some embodiments, the IC₅₀ of a compound may be less than about 50, 45, 40, 35, 30, 25, 20, 15, 10, 5, or about 1 μM. In other embodiments, the IC₅₀ of a compound may be less than about 1, 0.9, 0.8, 0.7, 0.6, 0.5, 0.4, 0.3, 0.2, or about 0.1 μM. In preferred embodiments, the IC₅₀ of a compound may be less than about 1 μM. In other preferred embodiments, the IC₅₀ of a compound may be about 0.9, 0.89, 0.88, 0.87, 0.86, 0.85, 0.84, 0.83, 0.82, 0.81, 0.8, 0.79, 0.78, 0.77, 0.76, 0.75, 0.74, 0.73, 0.72, 0.71, 0.7, 0.69, 0.68, 0.67, 0.66, 0.65, 0.64, 0.63, 0.62, 0.61, 0.6, 0.59, 0.58, 0.57, 0.56, 0.55, 0.54, 0.53, 0.52, 0.51, 0.5, 0.49, 0.48, 0.47, 0.46, 0.45, 0.44, 0.43, 0.42, 0.41, 0.4, 0.39, 0.38, 0.37, 0.36, 0.35, 0.34, 0.33, 0.32, 0.31, or about 0.3 μM. In exemplary embodiments, the IC₅₀ of a compound may be about 0.75, 0.74, 0.73, 0.72, 0.71, 0.7, 0.69, 0.68, 0.67, 0.66, 0.65, 0.64, 0.63, 0.62, 0.61, 0.6, 0.59, 0.58, 0.57, 0.56, 0.55, 0.54, 0.53, 0.52, 0.51, or about 0.5 μM.

In some embodiments, compounds of the invention specifically inhibit the activity of EndoG endonuclease. For instance, a compound of the invention may be about 1, 2, 3, 4, or 5 orders of magnitude more active against EndoG than against other endonucleases such as DNase I. In some embodiments, compounds of the invention are about two orders of magnitude more active against EndoG than against DNase I.

The IC₅₀ of a compound of the invention against EndoG may also be about 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, or about 200 times lower than the IC₅₀ of the compound against DNase I. In some embodiments the IC₅₀ of a compound of the invention against EndoG is about 10, 11, 12, 13, 14, 15, 16, 17, 18, 19, 20, 21, 22, 23, 24, or about 25 times lower than the IC₅₀ of the compound against DNase I. In other embodiments the IC₅₀ of a compound of the invention against EndoG is about 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, or about 125 times lower than the IC₅₀ of the compound against DNase I.

Compounds of the invention may also be used to modulate transcription and alternative splicing of nucleic acid sequences encoding DNase I by inhibiting EndoG. A skilled artisan will appreciate that in addition to cleaving damaged DNA, EndoG also preferentially cleaves noncanonical structures of DNA, triplex DNA, and R-loops that appear during transcription. As such, when compounds of the invention inhibit EndoG activity in a cell expressing DNase I, compounds may also inhibit expression of DNase I by inhibiting transcription and/or modulating alternative splicing of nucleic acids encoding DNase I. In some embodiments, a compound of the invention modulates alternative splicing of DNase I. In some embodiments, a compound of the invention promotes or increases expression of full-size, mature DNase I. In some embodiments, a compound of the invention reduces or decreases expression of the Δ4 DNase I isoform.

### (c) Helicase Inhibition

Compounds of the invention are capable of inhibiting helicase. As will be recognized by one of skill in the art, helicases are enzymes that unwind nucleic acid structures including DNA and RNA. In particular, compounds of the invention are capable of inhibiting viral helicase, such as NS3 helicases.

Any method of measuring helicase activity may be used to measure inhibition of helicase activity by compounds of the invention. In general, methods of measuring helicase may include fluorescent methods that show helicase-catalyzed displacement of a fluorescently-labeled oligonucleotide. As will be recognized by those of skill in the art, methods of measuring helicase activity can and will vary depending on the type of helicase, and whether the activity is measured in vitro, in vivo, or ex vivo.

Such assays can be used to determine the IC₅₀ for a compound. In some embodiments, the IC₅₀ of a compound may be less than about 50, 45, 40, 35, 30, 25, 20, 15, 10, 5, or about 1 μM. In other embodiments, the IC₅₀ of a compound may be less than about 1, 0.9, 0.8, 0.7, 0.6, 0.5, 0.4, 0.3, 0.2, or about 0.1 μM. In preferred embodiments, the IC₅₀ of a compound may be less than about 30 μM. In other preferred embodiments, the IC₅₀ of a compound may be about 15, about 16, about 17, about 18, about 19, about 20, about 21, about 22, about 23, about 24, about 25, about 26, about 27, about 28, about 29, about 30, about 31 about 32, about 33 about 34, about 35, about 36, or about 40 μM. In exemplary embodiments, the IC₅₀ of a compound may be less than about 30, less than about 29, less than about 28, less than about 27, less than about 26, less than about 25, less than about 24, less than about 23, less than about 22, less than about 21, or less than about 20.

In some embodiments, the compounds of the invention specifically inhibit the activity of viral helicases. For instance, a compound of the invention may be about 1, 2, 3, 4, or 5 orders of magnitude more active against viral helicase than human helicases. In other embodiments, the compound of the invention may be 1, 2, 3, 4, or 5 times more active against NS3 helicase than other helicases.

### II. Process for Making Compounds

As will be appreciated by a skilled artisan, the synthetic route used to produce compounds comprising Formula (I) can and will vary without departing from the scope of the invention. In one aspect of the invention, compounds comprising Formula (I) may be made in accordance with Reaction Scheme 1 shown below. Referring to Reaction Scheme 1, a compound comprising Formula (I) may be made via aldol condensation of compound A and compound B to produce a compound of the invention comprising Formula (I).

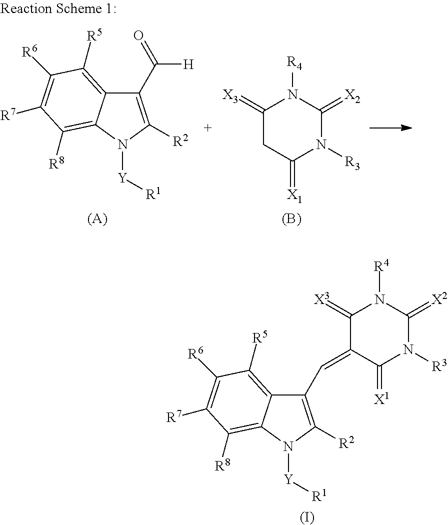

wherein:
X¹, X², X³, Y, R¹, R², R³, R⁴, R⁵, R⁶, R⁷, and R⁸ are as described in Section I.
   (a) Synthesis of Formula (I) from Compound (A)

In one embodiment, the disclosure provides a method for making the compound of Formula (I). The method comprises contacting a compound of Formula (I) with a compound of Formula (B). The compounds of Formula (A) and (B) have the above structures, and may be substituted as described in Section (I).

The mole to mole ratio of the compound comprising Formula (A) to the compound comprising Formula (B) can range over different embodiments of the invention. In one embodiment, the ratio of the compound comprising Formula (A) to the compound comprising Formula (B) varies from about 0.9:1 to about 1:10. In some embodiments, the mole to mole ratio of the compound comprising Formula (A) to the compound comprising Formula (B) is about 1:1 to about 1:1.5. In various embodiments, the mole to mole ratio of the compound comprising Formula (A) to the compound comprising Formula (B) is about 1:1, about 1:1.1, about 1:1.2, about 1:1.3, about 1:1.4, or about 1:1.5. In an exemplary embodiment, the mole to mole ratio of the compound comprising Formula (A) to the compound comprising Formula (B) is 1:1.

The reaction is preferably carried out in a solvent and is more preferably carried out in an organic solvent. The solvent may be chosen without limitation from including alkane and substituted alkane solvents (including cycloalkanes) alcohol solvents, halogenated solvents, aromatic hydrocarbons, esters, ethers, ketones, and combinations thereof. Non-limiting examples of suitable organic solvents are acetonitrile, acetone, allyl alcohol, benzene, butyl acetate, chlorobenzene, chloroform, chloromethane, cyclohexane, cyclopentane, dichloromethane (DCM), dichloroethane, diethyl ether, dimethoxyethane (DME), dimethylformamide (DMF), dimethyl sulfoxide (DMSO), dioxane, ethanol, ethyl acetate, ethylene dichloride, ethylene bromide, formic acid, fluorobenzene, heptane, hexane, isobutylmethylketone, isopropanol, isopropyl acetate, N-methylpyrrolidone, methanol, methylene bromide, methylene chloride, methyl iodide, methylethylketone, methyltetrahydrofuran, pentyl acetate, propanol, n-propyl acetate, sulfolane, tetrahydrofuran (THF), tetrachloroethane, toluene, trichloroethane, water, xylene and combinations thereof. In exemplary embodiments, the solvent is an alcohol solvent. In one preferred embodiment, the solvent is methanol.

The amount of time over which the reaction is conducted may also vary within different embodiments. In some embodiments, the reaction may be conducted over a period of 2 hours to 8 hours. In particular embodiments, the reaction is carried out for about 2 hours, about 3 hours, about 4 hours, about 5 hours, about 6 hours, about 7 hours, or about 8 hours. In preferred embodiments, the reaction is conducted for about 4 hours.

The temperature may vary over different embodiments, in some embodiments the temperature may range from about 50° C. to about 120° C. In particular embodiments the temperature may range from about 50° C. to about 60° C., from about 60° C. to about 70° C., from about 70° C. to about 80° C.from about 80° C. to about 90° C., from about 90° C. to about 100° C., from about 100° C. Without 110° C., or from about 110° C. to about 120° C.

The synthesized compounds may be used in their crude form or they may be purified. The compounds may be purified by any suitable method known in the art including through column chromatography, crystallization, distillation, extraction, and the like. In one preferred embodiment, the compounds are recrystallized from a solvent. The purity and identity of the compounds may be verified through X-ray crystallography, ¹H NMR, or ¹³C NMR, for example.

Other methods of aldol condensation are known in the art, and may be as described in, for example, Singh et al., 2009, 19:3054-3058, which is incorporated herein by reference in its entirety. Compound (I) may be synthesized as described in the Examples.

(b) Synthesis of Compound (VII) from Compound 4

In still another aspect, the invention provides a process for producing compound (VII) from the compound comprising Formula (C). The process comprises Step A and Step B as shown below:

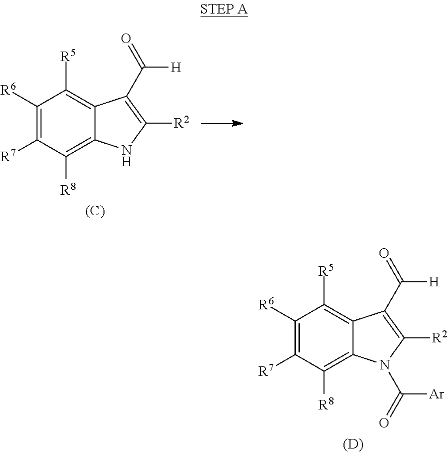

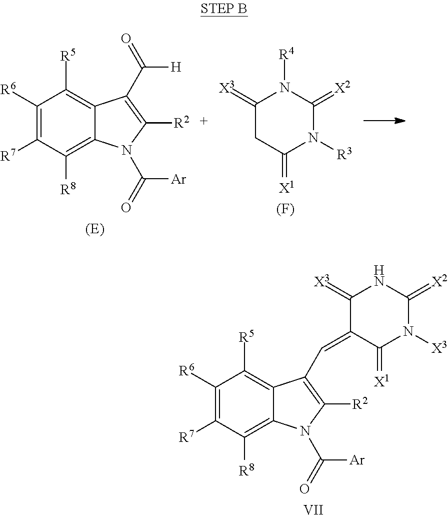

The transformation from the compound comprising Formula (C) to the compound comprising Formula (5) is generally conducted under phase-transfer catalytic (PTC) conditions. In one embodiment, the compound comprising Formula (C) is reacted with simple and substituted aroyl halides in the presence of a triethyl benzyl ammonium chloride (TEBA) catalyst to facilitate the transformation from the compound comprising Formula (C) to the compound comprising Formula (D). Any phase transfer catalyst can be used to accomplish this step, including, but not limited to quaternary ammonium salts, quaternary phosphonium salts, tertiary amines, quaternary arsonium salts, polyethylene glycols, cryptates, crown ethers. The particular phase catalyst may be chosen from, but is not limited to methyltrioctylammonium chloride, tetrabutylammonium bromide, tetrabutylammonium hydrogen sulfate, methyltributylammonium chloride, benzyltriethyl ammonium chloride, triethylamine, tributylamine, trioctylamine, tetrabutylphosphonium bromide, hexadecyltributlyphosphonium bromide, tetraphenylphosphonium bromide, 18-crown-6, dibenzo-18-crown-6, benzo-15-crown-5, polyethylene glycol with a molecular weight in the range of 300 to 3000, the dimethyl and dibutyl ethers of such polyethylene glycols, and tris(3,6-dioxaheptyl)amine (also known as TDA-1).

The phase transfer medium is generally consists of water and a polar solvent that is immiscible with water. In one preferred embodiment, the solvent for phase transfer catalysis is a mixture of dichloromethane and aqueous NaOH.

The compound comprising Formula (E) may be reacted to form a compound comprising Formula (I) as shown in Step B above, and as described in Section (II)(a).

### III. Pharmaceutical Composition

In another aspect, the invention encompasses a composition comprising compounds of the invention. Compounds may be as described in Section (I).

Compounds may be incorporated into pharmaceutical compositions suitable for administration. Such compositions typically comprise a compound of the invention and a pharmaceutically acceptable carrier. As used herein, the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with a compound, use thereof in the compositions is contemplated. Supplementary active compounds may also be incorporated into the compositions.

A pharmaceutical composition of the invention may be formulated to be compatible with its intended route of administration. Examples of routes of administration include parenteral, e.g., intravenous, intradermal, subcutaneous, oral (e.g., inhalation), transdermal (topical), transmucosal, and rectal administration. Formulation of pharmaceutical compositions is discussed in, for example, Hoover, John E., Remington's Pharmaceutical Sciences, Mack Publishing Co., Easton, Pa. (1975), and Liberman, H. A. and Lachman, L., Eds., Pharmaceutical Dosage Forms, Marcel Decker, New York, N.Y. (1980).

Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH may be adjusted with acids or bases, such as hydrochloric acid or sodium hydroxide. Parenteral preparations may be enclosed in ampoules, disposable syringes or multiple dose vials made of glass or plastic.

Pharmaceutical compositions suitable for injectable use may include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL (BASF; Parsippany, N.J.), or phosphate buffered saline (PBS). In all cases, a composition may be sterile and may be fluid to the extent that easy syringeability exists. A composition may be stable under the conditions of manufacture and storage and may be preserved against the contaminating action of microorganisms such as bacteria and fungi. The carrier may be a solvent or dispersion medium containing, for example, water, ethanol, polyol (for example, glycerol, propylene glycol, and liquid polyetheylene glycol, and the like), and suitable mixtures thereof. The proper fluidity may be maintained, for example, by the use of a coating such as lecithin, by the maintenance of the required particle size in the case of dispersion, and by the use of surfactants. Prevention of the action of microorganisms may be achieved by various antibacterial and antifungal agents, for example, parabens, chlorobutanol, phenol, ascorbic acid, thimerosal, and the like. In many cases, it may be preferable to include isotonic agents, for example, sugars, polyalcohols such as mannitol, sorbitol, or sodium chloride, in the composition. Prolonged absorption of the injectable compositions may be brought about by including in the composition an agent which delays absorption, for example, aluminum monostearate and gelatin.

Sterile injectable solutions may be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying, which yields a powder of the active ingredient plus any additional desired ingredient from a previously sterile-filtered solution thereof.

Oral compositions generally may include an inert diluent or an edible carrier. Oral compositions may be enclosed in gelatin capsules or compressed into tablets. For the purpose of oral therapeutic administration, the active compound may be incorporated with excipients and used in the form of tablets, troches, or capsules. Oral compositions may also be prepared using a fluid carrier for use as a mouthwash, wherein the compound in the fluid carrier is applied orally and swished and expectorated or swallowed. Pharmaceutically compatible binding agents and/or adjuvant materials may be included as part of the composition. The tablets, pills, capsules, troches, and the like, may contain any of the following ingredients, or compounds of a similar nature: a binder such as microcrystalline cellulose, gum tragacanth or gelatin; an excipient such as starch or lactose; a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. For administration by inhalation, the compounds are delivered in the form of an aerosol spray from a pressured container or dispenser which contains a suitable propellant, e.g., a gas such as carbon dioxide, or a nebulizer.

Systemic administration may also be by transmucosal or transdermal means. For transmucosal or transdermal administration, penetrants appropriate to the barrier to be permeated are used in the formulation. Such penetrants are generally known in the art, and may include, for example, for transmucosal administration, detergents, bile salts, and fusidic acid derivatives. Transmucosal administration may be accomplished through the use of nasal sprays or suppositories. For transdermal administration, the active compounds are formulated into ointments, salves, gels, or creams as generally known in the art. Compounds may also be prepared in the form of suppositories (e.g., with conventional suppository bases such as cocoa butter and other glycerides) or retention enemas for rectal delivery.

Compounds may be prepared with carriers that will protect a compound against rapid elimination from the body, such as a controlled release formulation, including implants and microencapsulated delivery systems. Biodegradable, biocompatible polymers may be used, such as ethylene vinyl acetate, polyanhydrides, polyglycolic acid, collagen, polyorthoesters, and polylactic acid. Methods for preparation of such formulations will be apparent to those skilled in the art. These may be prepared according to methods known to those skilled in the art, for example, as described in U.S. Pat. No. 4,522,811.

### IV. Method of Treating a Tumor

In another aspect, the invention encompasses a method of treating a tumor, the method comprising contacting a tumor cell with a composition comprising a compound of Formula (I). In general, a tumor cell that may be treated with a compound of the invention expresses a DNA repair polymerase. As used herein, “treating” refers to arresting the growth of a tumor or to decreasing the mass of the tumor. A composition of the invention may be formulated and administered to a subject by several different means as described in Section III.

### (a) Contacting a Cell

In some embodiments, a tumor cell may be contacted by a composition of the invention in vivo in a subject. The term “subject,” as used herein, refers to an animal. The subject may be an embryo, a juvenile, or an adult. Suitable animals include vertebrates such as mammals, birds, reptiles, amphibians, and fish. Examples of suitable mammals include without limit rodents, companion animals, livestock, and primates. Non-limiting examples of rodents include mice, rats, hamsters, gerbils, and guinea pigs. Suitable companion animals include, but are not limited to, cats, dogs, rabbits, hedgehogs, and ferrets. Non-limiting examples of livestock include horses, goats, sheep, swine, cattle, llamas, and alpacas. Suitable primates include, but are not limited to, humans, capuchin monkeys, chimpanzees, lemurs, macaques, marmosets, tamarins, spider monkeys, squirrel monkeys, and vervet monkeys. Non-limiting examples of birds include chickens, turkeys, ducks, and geese. An exemplary subject is a human.

In other embodiments, a cell contacted by a composition of the invention is an in vitro cell line. In some alternatives of the embodiments, the cell line may be a primary cell line that is not yet described. Methods of preparing a primary cell line utilize standard techniques known to individuals skilled in the art. In other alternatives, a cell line may be an established cell′ line. A cell line may be adherent or non-adherent, or a cell line may be grown under conditions that encourage adherent, non-adherent or organotypic growth using standard techniques known to individuals skilled in the art. A cell line may be contact inhibited or non-contact inhibited. Methods of culturing cell lines are known in the art.

In some embodiments, a method of treating a tumor as described herein may be administered in combination with other tumor treatment options such as surgery, chemotherapy, radiation therapy, or radiofrequency ablation. For instance, a method of the invention may comprise contacting a tumor cell with compounds and compositions of the invention in combination with one or more chemotherapeutic agents that act through the induction of DNA damage. While not wishing to be bound by theory, contacting a tumor cell with compounds and compositions of the invention in combination with a chemotherapeutic agent that act through the induction of DNA damage may reduce the resistance of cancers to the damaging effects of the chemotherapeutic agent by inhibiting DNA repair polymerases responsible for limiting the damaging effects of the chemotherapeutic agents.

Suitable chemotherapeutic agents that act through the induction of DNA damage may be selected from the group consisting of DNA synthesis inhibitors, mitotic inhibitors, alkylating agents, and nitrosoureas. Examples of DNA synthesis inhibitors include, but are not limited to, daunorubicin and adriamycin. Examples of mitotic inhibitors include paclitaxel, docetaxel, vinblastine, vincristine, and vinorelbine. Examples of antimetabolites include 5-fluorouracil, capecitabine, 6-mercaptopurine, methotrexate, gemcitabine, cytarabine (ara-C), fludarabine, pemetrexed, cytosine arabinoside, methotrexate, and aminopterin. Examples of alkylating agents include busulfan, cisplatin, carboplatin, chlorambucil, cyclophosphamide, ifosfamide, dacarbazine (DTIC), mechlorethamine, melphalan, and temozolomide. Examples of nitrosoureas include carmustine (BCNU) and iomustine (CCNU). Examples of anthracyclines include daunorubicin, doxorubicin, epirubicin, idarubicin, and mitoxantrone.

### (b) Suitable Tumor Cells

The method of the invention may be used to treat a tumor. As used herein, a “tumor” refers to a malignant or benign solid tumor or a tumor cell. The tumor may be primary or metastatic; early stage or late stage. Non-limiting examples of tumors that may be treated include acute lymphoblastic leukemia, acute myeloid leukemia, adrenocortical carcinoma, AIDS-related cancers, AIDS-related lymphoma, anal cancer, appendix cancer, astrocytomas (childhood cerebellar or cerebral), basal cell carcinoma, bile duct cancer, bladder cancer, bone cancer, brainstem glioma, brain tumors (cerebellar astrocytoma, cerebral astrocytoma/malignant glioma, ependymoma, medulloblastoma, supratentorial primitive neuroectodermal tumors, visual pathway and hypothalamic gliomas), breast cancer, bronchial adenomas/carcinoids, Burkitt lymphoma, carcinoid tumors (childhood, gastrointestinal), carcinoma of unknown primary, central nervous system lymphoma (primary), cerebellar astrocytoma, cerebral astrocytoma/malignant glioma, cervical cancer, childhood cancers, chronic lymphocytic leukemia, chronic myelogenous leukemia, chronic myeloproliferative disorders, colon cancer, cutaneous T-cell lymphoma, desmoplastic small round cell tumor, endometrial cancer, ependymoma, esophageal cancer, Ewing's sarcoma in the Ewing family of tumors, extracranial germ cell tumor (childhood), extragonadal germ cell tumor, extrahepatic bile duct cancer, eye cancers (intraocular melanoma, retinoblastoma), gallbladder cancer, gastric (stomach) cancer, gastrointestinal carcinoid tumor, gastrointestinal stromal tumor, germ cell tumors (childhood extracranial, extragonadal, ovarian), gestational trophoblastic tumor, gliomas (adult, childhood brain stem, childhood cerebral astrocytoma, childhood visual pathway and hypothalamic), gastric carcinoid, hairy cell leukemia, head and neck cancer, hepatocellular (liver) cancer, Hodgkin lymphoma, hypopharyngeal cancer, hypothalamic and visual pathway glioma (childhood), intraocular melanoma, islet cell carcinoma, Kaposi sarcoma, kidney cancer (renal cell cancer), laryngeal cancer, leukemias (acute lymphoblastic, acute myeloid, chronic lymphocytic, chronic myelogenous, hairy cell), lip and oral cavity cancer, liver cancer (primary), lung cancers (non-small cell, small cell), lymphomas (AIDS-related, Burkitt, cutaneous T-cell, Hodgkin, non-Hodgkin, primary central nervous system), macroglobulinemia (Waldenström), glioblastomas, malignant fibrous histiocytoma of bone/osteosarcoma, medulloblastoma (childhood), melanoma, intraocular melanoma, Merkel cell carcinoma, mesotheliomas (adult malignant, childhood), metastatic squamous neck cancer with occult primary, mouth cancer, multiple endocrine neoplasia syndrome (childhood), multiple myeloma/plasma cell neoplasm, mycosis fungoides, myelodysplastic syndromes, myelodysplastic/myeloproliferative diseases, myelogenous leukemia (chronic), myeloid leukemias (adult acute, childhood acute), multiple myeloma, myeloproliferative disorders (chronic), nasal cavity and paranasal sinus cancer, nasopharyngeal carcinoma, neuroblastoma, non-Hodgkin lymphoma, non-small cell lung cancer, oral cancer, oropharyngeal cancer, osteosarcoma/malignant fibrous histiocytoma of bone, ovarian cancer, ovarian epithelial cancer (surface epithelial-stromal tumor), ovarian germ cell tumor, ovarian low malignant potential tumor, pancreatic cancer, pancreatic cancer (islet cell), paranasal sinus and nasal cavity cancer, parathyroid cancer, penile cancer, pharyngeal cancer, pheochromocytoma, pineal astrocytoma, pineal germinoma, pineoblastoma and supratentorial primitive neuroectodermal tumors (childhood), pituitary adenoma, plasma cell neoplasia, pleuropulmonary blastoma, primary central nervous system lymphoma, prostate cancer, rectal cancer, renal cell carcinoma (kidney cancer), renal pelvis and ureter transitional cell cancer, retinoblastoma, rhabdomyosarcoma (childhood), salivary gland cancer, sarcoma (Ewing family of tumors, Kaposi, soft tissue, uterine), Sezary syndrome, skin cancers (nonmelanoma, melanoma), skin carcinoma (Merkel cell), small cell lung cancer, small intestine cancer, soft tissue sarcoma, squamous cell carcinoma, squamous neck cancer with occult primary (metastatic), stomach cancer, supratentorial primitive neuroectodermal tumor (childhood), T-Cell lymphoma (cutaneous), testicular cancer, throat cancer, thymoma (childhood), thymoma and thymic carcinoma, thyroid cancer, thyroid cancer (childhood), transitional cell cancer of the renal pelvis and ureter, trophoblastic tumor (gestational), unknown primary site (adult, childhood), ureter and renal pelvis transitional cell cancer, urethral cancer, uterine cancer (endometrial), uterine sarcoma, vaginal cancer, visual pathway and hypothalamic glioma (childhood), vulvar cancer, Waldenström macroglobulinemia, and Wilms tumor (childhood).

In a preferred embodiment, the tumor may be a brain tumor, or a skin tumor.

### V. Methods of Inhibiting Endonuclease

In another aspect, the invention encompasses methods of using compounds of the invention or compositions comprising compounds of the invention in vitro, ex vivo, in vivo and in situ. Non-limiting examples of uses for compounds of the invention are described above in Section I. In general, the method comprises contacting a cell with a composition comprising a compound of Formula (I) under conditions effective to allow for internalization of the compound into the cell. In general, a cell that may be treated with a compound of the invention expresses EndoG. Advantageously, compounds of the invention are typically cell-permeable, given their low molecular weight. Also contemplated are derivatives of compounds of Formula (I) that have an increased ability to be internalized. Methods for promoting cell internalization are not in the art and include, but are not limited to, conjugation to a cell penetrating peptide (see for example Methods Mol Biol (2011) 683: 535-51, hereby incorporated by reference in its entirety). In a preferred embodiment, the compound is PNR-3-80. In another preferred embodiment, the compound is PNR-3-82.

In some embodiments, a cell may be contacted by a composition of the invention in vivo in a subject. Stated another way, a composition of the invention may be administered to a subject. A suitable amount of the composition should be administered to a subject. Though the amount can and will vary depending on several factors (for example, type of subject, route of administration, etc.), a suitable amount may be determined by experimentation. The term “subject,” as used herein, refers to an animal. The subject may be an embryo, a juvenile, or an adult. Suitable animals include vertebrates such as mammals, birds, reptiles, amphibians, and fish. Examples of suitable mammals include, without limit, rodents, companion animals, livestock, and primates. Non-limiting examples of rodents include mice, rats, hamsters, gerbils, and guinea pigs. Suitable companion animals include, but are not limited to, cats, dogs, rabbits, hedgehogs, and ferrets. Non-limiting examples of livestock include horses, goats, sheep, swine, cattle, llamas, and alpacas. Suitable primates include, but are not limited to, humans, capuchin monkeys, chimpanzees, lemurs, macaques, marmosets, tamarins, spider monkeys, squirrel monkeys, and vervet monkeys. Non-limiting examples of birds include chickens, turkeys, ducks, and geese. An exemplary subject is a human.

In other embodiments, a cell contacted by a composition of the invention is an in vitro cell line. In some alternatives of the embodiments, the cell line may be a primary cell line that is not yet described. Methods of preparing a primary cell line utilize standard techniques known to individuals skilled in the art. In other alternatives, a cell line may be an established cell line. A cell line may be adherent or non-adherent, or a cell line may be grown under conditions that encourage adherent, non-adherent or organotypic growth using standard techniques known to individuals skilled in the art. A cell line may be contact inhibited or non-contact inhibited. One skilled in the art will appreciate that any known cell type that can be cultured in vitro, even for a limited time, may be used in the method of the invention. Methods of culturing cell lines are known in the art. In certain embodiments, a cell may be processed into a cell lysate before contact with a composition of the invention. Typically, contacting an in vitro cell line with a composition of the invention

In other embodiments, a cell may be contacted by a composition of the invention ex vivo or in situ in a tissue sample or organ obtained from a subject. Non-limiting examples of suitable tissues includes connective tissue, muscle tissue, nervous tissue, and epithelial tissue. Non-limiting examples of suitable organs include organs of the cardiovascular system, digestive system, the endocrine system, the excretory system, the immune system, the nervous system, the reproductive system, the respiratory system. Tissue and organ samples may be cultured in vitro or ex vivo. They may also be biopsy samples or otherwise removed from a subject. In certain embodiments, a tissue or organ sample may be homogenized before contact with a composition of the invention.

In some embodiments, the invention encompasses a method of protecting a cell from various injuries that may induce cell death, the method comprising contacting a cell expressing EndoG with a composition comprising a compound of Formula (I). As used herein, “protecting a cell” refers to inhibiting cell death in a cell that has sustained an injury that may induce cell death. As will be recognized by individuals skilled in the art, cells may die through either of two distinct processes: necrosis or apoptosis. Necrosis is death due to unexpected and accidental cell damage and begins by cell swelling, followed by the appearance of holes in the plasma membrane and spilling of intracellular materials into the surrounding environment, causing inflammation. Apoptosis is programmed cell death and may not cause inflammation. Apoptosis is a process by mediated by an intracellular program and may include blebbing, cell shrinkage, nuclear fragmentation, chromatin condensation, and chromosomal DNA fragmentation. Remnants of apoptotic cells are rapidly engulfed by their neighbors and removed. A method of the invention may be used to protect a cell from various injuries that may induce any type of cell death, provided EndoG participates in the cell death process. In some embodiments, a method of the invention may be used to protect a cell from injuries that may induce necrotic cell death. In preferred embodiments, a method of the invention may be used to protect a cell from injuries that may induce programmed cell death.

Non-limiting examples of injuries that may induce cell death include chemical poisoning, drug poisoning, radiation, hypoxia, physical injury, and DNA-damaging and cell death-inducing chemotherapeutics that are used to promote cell death in cancer cells. Injuries that induce cell death may also arise spontaneously. In some embodiments, a method of the invention may protect a cell that has sustained an injury from chemical poisoning. In other embodiments, a method of the invention may protect a cell that has sustained an injury from drug poisoning. In yet other embodiments, a method of the invention may protect a cell that has sustained an injury from cell death-inducing chemotherapeutics that are used to promote cell death in cancer cells. In still other embodiments, a method of the invention may protect a cell that has sustained a spontaneous injury that may induce cell death.

In some embodiments, a method of protecting a cell as described herein may comprise administering a composition of the invention in combination with other treatment options. For instance, a method of protecting a cell as described herein may comprise administering a composition of the invention in combination with tumor treatment options such as surgery, chemotherapy, radiation therapy, or radiofrequency ablation. For instance, a method of the invention may comprise contacting a tumor cell, or a tissue comprising a tumor cell, with compounds and compositions of the invention in combination with one or more cell death-inducing chemotherapeutic agents. While not wishing to be bound by theory, contacting a tissue comprising a tumor cell with compounds and compositions of the invention in combination with a cell death-inducing chemotherapeutic agent that acts through the induction of DNA damage may protect normal cells surrounding the tumor cell, and may protect the normal cells from the damaging effects of the chemotherapeutic agent by inhibiting cell death.

Suitable cell death-inducing chemotherapeutics may be selected from the group consisting of DNA synthesis inhibitors, mitotic inhibitors, alkylating agents, and nitrosoureas. Examples of DNA synthesis inhibitors include, but are not limited to, daunorubicin and adriamycin. Examples of mitotic inhibitors include paclitaxel, docetaxel, vinblastine, vincristine, and vinorelbine. Examples of antimetabolites include 5-fluorouracil, capecitabine, 6-mercaptopurine, methotrexate, gemcitabine, cytarabine (ara-C), fludarabine, pemetrexed, cytosine arabinoside, methotrexate, and aminopterin. Examples of alkylating agents include busulfan, cisplatin, carboplatin, chlorambucil, cyclophosphamide, ifosfamide, dacarbazine (DTIC), mechlorethamine, melphalan, and temozolomide. Examples of nitrosoureas include carmustine (BCNU) and iomustine (CCNU). Examples of anthracyclines include daunorubicin, doxorubicin, epirubicin, idarubicin, and mitoxantrone.

In some embodiments, the invention encompasses a method of modulating alternative splicing of DNase I, the method comprises contacting a cell expressing EndoG with a composition comprising a compound of Formula (I). In other embodiments, the invention encompasses a method of decreasing expression of Δ4DNase I, the method comprises contacting a cell expressing EndoG with a composition comprising a compound of Formula (I).

### VI. Methods of Inhibiting Helicase

In another aspect, the invention encompasses methods of using compounds of the invention or compositions comprising compounds of the invention in vitro, ex vivo, in vivo and in situ. Non-limiting examples of uses for compounds of the invention are described above in Section I. In general, the method comprises contacting a cell with a composition comprising a compound of Formula (I) under conditions effective to allow for internalization of the compound into the cell. In general, a cell that may be treated with a compound of the invention expresses a helicase. Advantageously, compounds of the invention are typically cell-permeable, given their low molecular weight. Also contemplated are derivatives of compounds of Formula (I) that have an increased ability to be internalized. Methods for promoting cell internalization are not in the art and include, but are not limited to, conjugation to a cell penetrating peptide (see for example Methods Mol Biol (2011) 683: 535-51, hereby incorporated by reference in its entirety). In an preferred embodiment, the compound is PNR-3-80. In another preferred embodiment, the compound is PNR-3-82.

In some embodiments, a cell may be contacted by a composition of the invention in vivo in a subject. Stated another way, a composition of the invention may be administered to a subject. A suitable amount of the composition should be administered to a subject. Though the amount can and will vary depending on several factors (for example, type of subject, route of administration, etc.), a suitable amount may be determined by experimentation. The term “subject,” as used herein, refers to an animal. The subject may be an embryo, a juvenile, or an adult. Suitable animals include vertebrates such as mammals, birds, reptiles, amphibians, and fish. Examples of suitable mammals include, without limit, rodents, companion animals, livestock, and primates. Non-limiting examples of rodents include mice, rats, hamsters, gerbils, and guinea pigs. Suitable companion animals include, but are not limited to, cats, dogs, rabbits, hedgehogs, and ferrets. Non-limiting examples of livestock include horses, goats, sheep, swine, cattle, llamas, and alpacas. Suitable primates include, but are not limited to, humans, capuchin monkeys, chimpanzees, lemurs, macaques, marmosets, tamarins, spider monkeys, squirrel monkeys, and vervet monkeys. Non-limiting examples of birds include chickens, turkeys, ducks, and geese. An exemplary subject is a human.

In other embodiments, a cell contacted by a composition of the invention is an in vitro cell line. In some alternatives of the embodiments, the cell line may be a primary cell line that is not yet described. Methods of preparing a primary cell line utilize standard techniques known to individuals skilled in the art. In other alternatives, a cell line may be an established cell line. A cell line may be adherent or non-adherent, or a cell line may be grown under conditions that encourage adherent, non-adherent or organotypic growth using standard techniques known to individuals skilled in the art. A cell line may be contact inhibited or non-contact inhibited. One skilled in the art will appreciate that any known cell type that can be cultured in vitro, even for a limited time, may be used in the method of the invention. Methods of culturing cell lines are known in the art. In certain embodiments, a cell may be processed into a cell lysate before contact with a composition of the invention. Typically, contacting an in vitro cell line with a composition of the invention

In other embodiments, a cell may be contacted by a composition of the invention ex vivo or in situ in a tissue sample or organ obtained from a subject. Non-limiting examples of suitable tissues includes connective tissue, muscle tissue, nervous tissue, and epithelial tissue. Non-limiting examples of suitable organs include organs of the cardiovascular system, digestive system, the endocrine system, the excretory system, the immune system, the nervous system, the reproductive system, the respiratory system. Tissue and organ samples may be cultured in vitro or ex vivo. They may also be biopsy samples or otherwise removed from a subject. In certain embodiments, a tissue or organ sample may be homogenized before contact with a composition of the invention.

In some embodiments, the invention encompasses a method of inhibiting helicase in a cell expressing helicase with a composition comprising a compound of Formula (I).

In still another embodiment, the invention encompasses methods of treating or preventing hepatitis C virus in a subject. The method involves vaccinating a subject with a composition of the invention as described in Section I. Prevention, as used herein, means a lowered risk for developing hepatitis C for a treatment group of subjects than for a control group of subjects.

Vaccine compositions of the invention may be formulated into pharmaceutical compositions and administered by a number of different means that may deliver a therapeutically effective dose. Such compositions may be administered orally, parenterally, by inhalation spray, rectally, intradermally, transdermally, or topically in dosage unit formulations containing conventional nontoxic pharmaceutically acceptable carriers, adjuvants, and vehicles as desired. Topical administration may also involve the use of transdermal administration such as transdermal patches or iontophoresis devices. The term parenteral as used herein includes subcutaneous, intravenous, intramuscular, or intrasternal injection, or infusion techniques. In preferred embodiments, vaccine compositions of the invention are formulated for intramuscular administration. Formulation of vaccines is discussed in, for example, Hoover, John E., Remington's Pharmaceutical Sciences, Mack Publishing Co., Easton, Pa. (1975), and Liberman, H. A. and Lachman, L., Eds., Pharmaceutical Dosage Forms, Marcel Decker, New York, N.Y. (1980).

Vaccine compositions suitable for injectable use may include sterile aqueous solutions (where water soluble) or dispersions and sterile powders for the extemporaneous preparation of sterile injectable solutions or dispersion. Suitable carriers include physiological saline, bacteriostatic water, Cremophor EL (BASF; Parsippany, N.J.), or phosphate buffered saline (PBS). In all cases, a composition may be sterile and may be fluid to the extent that easy syringeability exists. A composition may be stable under the conditions of manufacture and storage and may be preserved against the contaminating action of microorganisms such as bacteria and fungi. The carrier may be a solvent or dispersion medium containing, for example, water, ethanol, polyol (for example, glycerol, propylene glycol, and liquid polyetheylene glycol, and the like), and suitable mixtures thereof. The proper fluidity may be maintained, for example, by the use of a coating such as lecithin, by the maintenance of the required particle size in the case of dispersion, and by the use of surfactants. Prevention of the action of microorganisms may be achieved by various antibacterial and antifungal agents, for example, parabens, chlorobutanol, phenol, ascorbic acid, thimerosal, and the like. In many cases, it may be preferable to include isotonic agents, for example, sugars, polyalcohols such as mannitol, sorbitol, or sodium chloride, in the composition. Prolonged absorption of the injectable compositions may be brought about by including in the composition an agent which delays absorption, for example, aluminum monostearate and gelatin.

Sterile injectable solutions may be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying, which yields a powder of the active ingredient plus any additional desired ingredient from a previously sterile-filtered solution thereof.

### DEFINITIONS

The terms “DNA repair polymerase” and “DNA damage repair polymerase enzyme” are used herein interchangeably to describe polymerase enzymes expressed in a cell in response to DNA damage.

Unless otherwise indicated, the alkyl groups described herein are preferably lower alkyl containing from one to eight carbon atoms in the principal chain and up to 20 carbon atoms. They may be straight or branched chain or cyclic and include methyl, ethyl, propyl, isopropyl, butyl, hexyl and the like.

Unless otherwise indicated, the alkenyl groups described herein are preferably lower alkenyl containing from two to eight carbon atoms in the principal chain and up to 20 carbon atoms. They may be straight or branched chain or cyclic and include ethenyl, propenyl, isopropenyl, butenyl, isobutenyl, hexenyl, and the like.

The terms “hydrocarbon” and “hydrocarbyl” as used herein describe organic compounds or radicals consisting exclusively of the elements carbon and hydrogen. These moieties include alkyl, alkenyl, and alkynyl moieties. These moieties also include alkyl, alkenyl, and alkynyl moieties substituted with other aliphatic or cyclic hydrocarbon groups, such as alkaryl, alkenaryl and alkynaryl. Unless otherwise indicated, these moieties preferably comprise 1 to 20 carbon atoms.

The term “aromatic” as used herein alone or as part of another group denotes optionally substituted homo- or heterocyclic aromatic groups. These aromatic groups are preferably monocyclic, bicyclic, or tricyclic groups containing from 6 to 14 atoms in the ring portion. The term “aromatic” encompasses the “aryl” and “heteroaryl” groups defined below.

The term “aryl” or “Ar” as used herein alone or as part of another group denote optionally substituted homocyclic aromatic groups, preferably monocyclic or bicyclic groups containing from 6 to 12 carbons in the ring portion, such as phenyl, biphenyl, naphthyl, substituted phenyl, substituted biphenyl or substituted naphthyl. Phenyl and substituted phenyl are the more preferred aryl.

The “substituted phenyl” moieties described herein are phenyl moieties which are substituted with at least one atom other than hydrogen. Exemplary substituents include one or more of the following groups: hydrocarbyl, substituted hydrocarbyl, hydroxy, protected hydroxy, acyl, acyloxy, alkoxy, alkenoxy, alkynoxy, aryloxy, halogen, amido, amino, cyano, ketals, acetals, esters and ethers.

The “substituted biphenyl” moieties described herein are biphenyl moieties which are substituted with at least one atom other than hydrogen. Exemplary substituents include one or more of the following groups: hydrocarbyl, substituted hydrocarbyl, hydroxy, protected hydroxy, acyl, acyloxy, alkoxy, alkenoxy, alkynoxy, aryloxy, halogen, amido, amino, cyano, ketals, acetals, esters and ethers.

The “substituted naphthyl” moieties described herein are naphthyl moieties which are substituted with at least one atom other than hydrogen. Exemplary substituents include one or more of the following groups: hydrocarbyl, substituted hydrocarbyl, hydroxy, protected hydroxy, acyl, acyloxy, alkoxy, alkenoxy, alkynoxy, aryloxy, halogen, amido, amino, cyano, ketals, acetals, esters and ethers.

The term “carbonyl” as used herein alone or as part of another group denotes a group comprising a carbon oxygen double bond.

The term “heteroatom” shall mean atoms other than carbon and hydrogen.

The terms “heterocyclo” or “heterocyclic” as used herein alone or as part of another group denote optionally substituted, fully saturated or unsaturated, monocyclic or bicyclic, aromatic or non-aromatic groups having at least one heteroatom in at least one ring, and preferably 5 or 6 atoms in each ring. The heterocyclo group preferably has 1 or 2 oxygen atoms and/or 1 to 4 nitrogen atoms in the ring, and is bonded to the remainder of the molecule through a carbon or heteroatom. Exemplary heterocyclo groups include heteroaromatics as described below. Exemplary substituents include one or more of the following groups: hydrocarbyl, substituted hydrocarbyl, hydroxy, protected hydroxy, acyl, acyloxy, alkoxy, alkenoxy, alkynoxy, aryloxy, halogen, amido, amino, cyano, ketals, acetals, esters and ethers.

The term “heteroaryl” as used herein alone or as part of another group denote optionally substituted aromatic groups having at least one heteroatom in at least one ring, and preferably 5 or 6 atoms in each ring. The heteroaryl group preferably has 1 or 2 oxygen atoms and/or 1 to 4 nitrogen atoms in the ring, and is bonded to the remainder of the molecule through a carbon.

The “substituted hydrocarbyl” moieties described herein are hydrocarbyl moieties which are substituted with at least one atom other than carbon, including moieties in which a carbon chain atom is substituted with a hetero atom such as nitrogen, oxygen, silicon, phosphorous, boron, sulfur, or a halogen atom.

The “alkoxy or arylalkoxy” moieties described herein may include methoxy, ethoxy, benzyloxy, and substituted benzyloxy.

The term “halogen” as used herein, alone or as part of another group, refers to chlorine, bromine, fluorine, and iodine.

The term “lower alkyl” as used herein refers to straight or branched chain alkyl radicals having in the range of about 1 up to 4 carbon atoms.

The term “DMSO” as used herein refers to dimethyl sulfoxide.

As various changes could be made in the above compounds, products and methods without departing from the scope of the invention, it is intended that all matter contained in the above description and in the examples given below, shall be interpreted as illustrative and not in a limiting sense.

### EXAMPLES

The following examples are included to demonstrate preferred embodiments of the disclosure. It should be appreciated by those of skill in the art that the techniques disclosed in the examples that follow represent techniques discovered by the inventors to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Introduction for Examples 1-4

Efficient DNA replication is a barrier to genomic instability. The process of replicating DNA in a timely manner is perturbed by both exogenous and endogenous processes. DNA adducts and/or natural replication fork barriers, such as G-quadruplex forming sequences, can impede progress by inhibiting the replication machinery. In these instances of perturbed replication, there are cellular mechanisms in place that recruit specialized polymerases to sites of replication stress. These so-called replication stress response (RSR) polymerases assist replication fork progression during S-phase and participate in post-replication repair events that occur during the G2/M transition at sites where the replisome has collapsed and ssDNA gaps persist. Replication stress is a hallmark of cancer and many existing chemo- and radiotherapies act to limit tumor growth primarily through the induction of DNA damage, which impairs DNA synthesis and repair. Moreover, recent studies have shown that in some tumors, such as highly malignant brain tumors, markers of replication stress are constitutively activated prior to treatment with genotoxic agents. Up-regulation of specialized RSR polymerases in these tumors may contribute to the progression of the disease by promoting increased genomic instability, as has been demonstrated by examination of clinical specimens and through in vitro experiments with the human Y-family DNA polymerase kappa (hpol κ).

As indicated above, anti-cancer treatments that use DNA damage as a means of eliminating tumor cells are often rendered ineffective through the stimulation of DNA repair mechanisms or through other acquired mutations, which result in resistance to the damaging effects of the compound. Another way of acquiring resistance to genotoxic agents is through pathways that allow the cell to tolerate the DNA damage by performing translesion DNA synthesis (TLS) past the offending lesion instead of performing repair. DNA damage tolerance pathways are utilized when DNA adducts are not repaired prior to S-phase or when the repair mechanism requires a specialized polymerase to complete the repair process (e.g., nucleotide excision repair of cross-linked DNA). TLS is an important part of the replication stress response mediated by the RSR-associated ATR/Chk1 kinase signaling pathway. The nature of TLS is to bypass lesions that are often incapable of forming normal Watson-Crick base pairs and as such, is generally thought to be somewhat error-prone. Thus, activation of TLS pathways in response to anti-cancer treatments can directly contribute to cell survival in the face of DNA damage and simultaneously produce mutations associated with the development of resistance. The ability to specifically target these processes in tumor cells could be of great potential benefit.

The enzymes primarily responsible for DNA adduct bypass include the Y-family DNA polymerases (pols). These specialized polymerases exhibit unique structural and functional properties that allow for the successful copying of DNA adducts, while also making them targets for small-molecule inhibitors. The mis-regulation and mutation of Y-family pols has been observed in many tumor types. Importantly, recent studies have shown that Y-family polymerases, particularly human DNA polymerase eta (hpol η), participate in mechanisms that promote resistance to anti-cancer treatments, such as cisplatin and doxorubicin. As described in the examples below, inhibitors of DNA polymerase activity were identified by utilizing a previously reported fluorescence-based assay that measures polymerase-catalyzed strand displacement, which is dependent upon nucleotidyl transfer by the enzyme. A targeted collection of over 300 compounds that were designed to target nucleic acid-interacting proteins and enzymes were screened. Of these 300 compounds, one of the more potent inhibitors of DNA polymerase activity was found to contain an indole thio-barbituric acid (ITBA) chemical structure. A number of ITBA derivatives were then prepared to assess structure-activity relationships and steady-state kinetic analysis of the compound to determine the mechanism of polymerase inhibition.

### Materials and Methods for Examples 1-4

Materials.

All chemicals were molecular biology grade or better. All dNTPs were purchased from GE Healthcare Life Sciences (Piscataway, N.J.). All oligonucleotides used in this work were synthesized by either Integrated DNA technologies (Coralville, Iowa) or Biosearch Technologies (San Diego, Calif.) and purified by the manufacturer using HPLC, with analysis by matrix-assisted laser desorption time-of-flight MS. The primer sequence used in the gel-based extension assays and inhibition assays was 5′(FAM-TTT)-GGG GGA AGG ATT C-3′ (SEQ ID NO. 1). The template DNA sequence used in the gel-based extension assays and inhibition assays was 5′-TCA CGG AAT CCT TCC CCC-3′ (SEQ ID NO. 2).

Expression and Purification of Recombinant Proteins.

The pBG101plasmid was used to prepare constructs encoding human DNA polymerases η (amino acids 1-437), ι (amino acids 26-446) and κ (amino acids 19-526). The pBG101 vector encodes a 6×-histidine tag and a glutathione transferase (GST) fusion protein upstream of the polymerase-encoding region. A protease recognition sequence (SEQ ID NO. 3: LEVLFQGP) just upstream of the polymerase insert allows cleavage of the N-terminal affinity tags during purification. All the human polymerases used in the study were expressed in Escherichia coli (strain BL21 DE3) and purified in an identical manner. Briefly, pBG101 vector encoding the polymerases just downstream of 6×-Histidine and GST-tags was transformed into E. coli cells (BL21 (DE3) strain). Cells were grown at 37° C. and 250 rpm for three hours (OD₆₀₀=0.5-0.6), followed by induction for three hours (37° C. and 250 rpm) by addition of isopropyl 13-D-1-thiogalactopyranoside (1 mM), and finally harvested by centrifugation. Buffer containing 50 mM Tris-HCl (pH 7.4), 0.5 M NaCl, 10% glycerol (v/v), 5 mM 13-mercaptoethanol (13-ME), lysozyme (1 mg/ml), and a protease inhibitor cocktail (Roche, Basel, Switzerland) was added to the harvested pellet. The suspension was sonicated and supernatant recovered from an ultracentrifugation step (35,000 g, 1 h, 4° C.). After the removal of cellular debris by ultracentrifugation, the resulting clear lysate was loaded onto a 5 mL HisTrap column (GE Healthcare Life Sciences) followed by washing the column sequentially with 50 mM Tris-HCl (pH 7.3 at 22° C.) buffer containing 0.5 M NaCl, 5 mM 13-ME, 10% glycerol and 20 mM imidazole to remove non-specifically bound proteins. The remaining bound proteins were then eluted using a linear gradient from 60 mM to 400 mM imidazole. The eluted proteins were loaded onto a 2 mL GSTrap column (GE Healthcare Life Sciences) in 25 mM HEPES (pH 7.5) buffer containing 0.1 M NaCl, 5 mM 13-ME, and 10% glycerol. Cleavage of the GST tag was performed on the bound proteins by injecting a solution containing the PreScission protease (GE Healthcare Life Sciences) onto the column and allowing it to incubate overnight at 4° C. The G ST-tag-free proteins were eluted in the GSTrap running buffer and concentrated using an Amicon spin concentrator (MilliPore). The purity of each polymerase was analyzed by SDS-polyacrylamide gel electrophoresis. The highly pure proteins were stored at −80° C. in the HEPES buffer (pH 7.5) containing 0.1 M NaCl, 5 mM 13-ME, and 30% glycerol. The model B-family DNApolymerases, Dpo1 and Dpo4, from Sulfolobus solfataricus were expressed and purified as described previously in the art. Human DNA polymerase beta (hpol 13) was purchased from Enzymax (Lexington, Ky.). HIV-1 RT was kindly provided by Prof. F. Peter Guengerich (Vanderbilt University School of Medicine).

Fluorescence-Based Assay to Screen for Inhibition of DNA Polymerase Activity.

A library of 320 compounds targeted against nucleic acid interacting proteins was screened for inhibition of polymerase activity using an assay that monitors fluorescence from a 5-carboxytetramethylrhodamine (TAMRA) labeled oligonucleotide. In order to prepare the DNA for the experiment, a TAMRA-labeled reporter (or displaced) strand (SEQ ID NO. 4: 5′-TTT TTT TTG C-TAMRA-3′) and unlabeled primer strand (SEQ ID NO. 5: 5′-TCA CCC TCGTAC GAC TCT T-3′) were annealed to a Black Hole Quencher (BHQ)-labeled template strand (SEQ ID NO. 6: 5′BHQ2-GCAAAAAAAAAA GAG TCG TAC GAG GGT GA-3′) in a solution containing 10 mM Tris (pH 8.0), 50 mM NaCl, 2 mM MgCl₂, and dH2O. The template (T), primer (P) and displaced strand (D) oligonucleotides were mixed in a 1:1.5:1.5 (T:P:D) molar ratio for annealing. After an incubation period of three minutes at 95° C., the DNA was allowed to slowly cool to room temperature overnight.

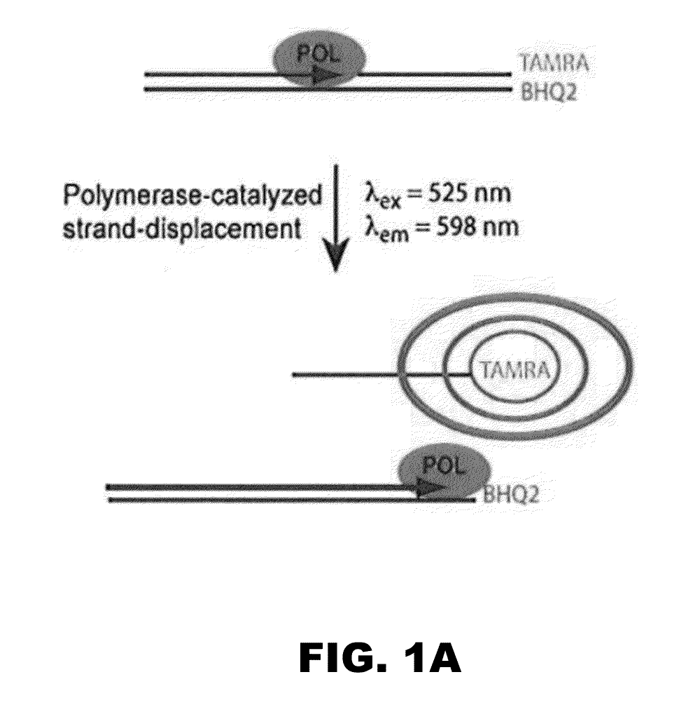

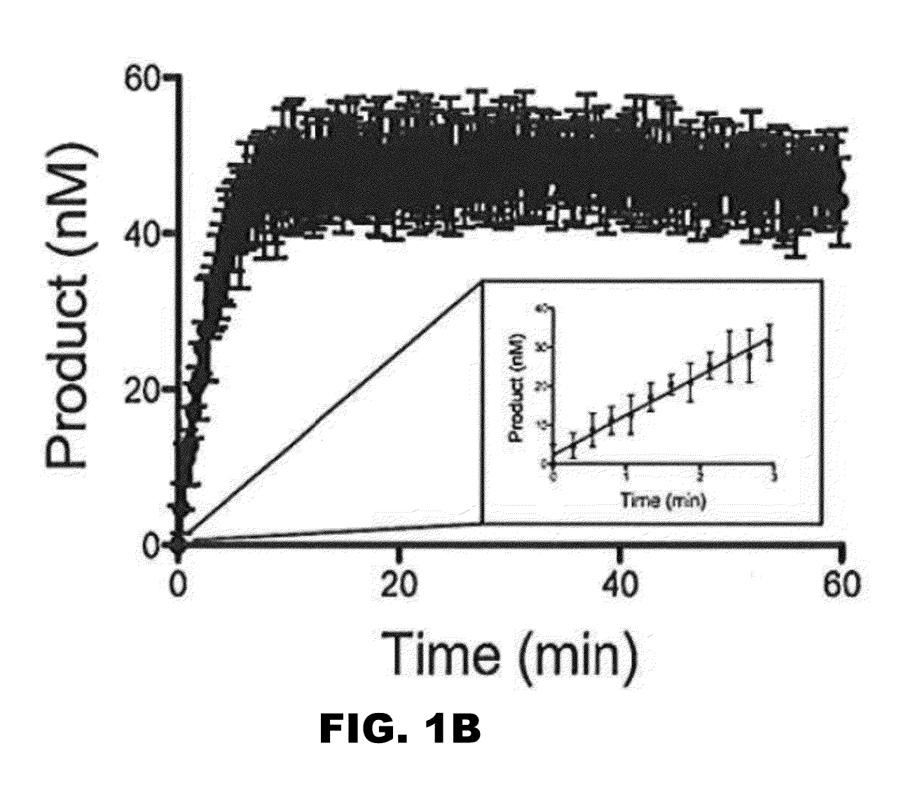

The fluorescence-based assay used to screen for polymerase inhibitors measures polymerase-catalyzed displacement of a TAMRA-labeled oligonucleotide (FIG. 1). For the initial screen, the experimental setup included 50 nM hpol η), 50 nM DNA, 6 μM compound, 100 μM dTTP and 1 mM MgCl₂. The reactions were performed in 50 mM Tris (pH 8.0) buffer containing 40 mM NaCl, 2 mM dithiothreitol, and 0.01% (v/v) Tween-20. The concentration of dimethyl sulfoxide (DMSO) was 3.5% (v/v) for the initial screen. The enzyme, the compounds (including a DMSO control) and dTTP were combined with the reaction buffer in individual wells of each half-plate and allowed to incubate for 5-10 minutes. The DNA substrate was subsequently added to initiate the reaction and the plate was read immediately using a BioTek SynergyH4 plate reader (λₑₓ=525 nm, λₑₘ=598 run). The final reaction volume was 200 μL. Fluorescence was monitored for 90 minutes for most reactions. The initial portion of the velocity curve was analyzed by linear regression to calculate an observed rate of product formation. For each data set, eight DMSO control experiments were averaged to obtain our measure of 100% activity. Rates of product formation in the presence of each compound were then divided by the rate of the DMSO control to produce a relative measure of polymerase activity (0 to 1, with 1 being no inhibition).

Compounds.

A series of novel substituted N-alkyl and N-aroyl-1H-indol-3-yl)methylene)-barbiturates or 2-thiobarbiturates indomethacin analogs were synthesized by aldol condensation of the appropriate N-substituted, simple and 2-methyl indole-3-carboxaldehydes with barbituric acid and thiobarbituric acid and its related compounds. The structure and purity of these derivatives was verified by ¹H and ¹³C-NMR spectroscopy. The indole-3-aldehyde and barbituric acid or thiobarbituric acid are stirred in methanol at room temperature for about 4-6 hours. The obtained yellow solid is filtered, washed with methanol, and dried under reduced pressure to afford the desired product.

Gel-Based Assay Measuring DNA Polymerase Activity.

In order to provide a second measure of enzyme inhibition, polymerase extension assays were performed. Briefly, hpol η (2 nM) was pre-incubated with FAM-13/18-mer primer-template DNA (100 μM) and either DMSO (final concentration=10%) or compound (6 μM, 13 μM and 60 μM; maintaining 10% DMSO for all experiments). Polymerase catalysis was initiated by the addition of dNTP (1 mM) and MgCl₂ (5 mM). The reaction was allowed to proceed at 37° C. for varying times and then terminated by the addition of 5 μL aliquots of the reaction mix to 25 μL of the quench solution (20 mM EDTA, 95% (v/v) formamide and 0.1% (w/v) bromophenol blue). The samples were separated using a 16% polyacrylamide/7M urea gel and the products analyzed using a Typhoon imager and ImageQuant™ software (GE Healthcare Life Sciences).

Determination of IC₅₀ Values for Individual DNA Polymerases.

In order to determine the IC₅₀ value for each enzyme, the fluorescence-based polymerase assay was repeated with increasing concentrations of inhibitor. The conditions varied slightly for each enzyme. With the exception of hpol ι, all IC₅₀ experiments were performed in 50 mM Tris HCl (pH 8.0) buffer containing 1 mM MgCl₂, 0.1 mM dTTP, 40 mM NaCl, 2 mM DTT, 0.01% (v/v) Tween-20 and 10% (v/v) DMSO. For experiments with hpol ι, KCl was substituted for NaCl and 0.25 mM MnCl₂ was substituted for MgCl₂. The concentration of inhibitor was 0, 1, 5, 10, 20, 30, 50, 75 and 100 μM. The enzyme and DNA concentrations were as follows: 10 nM hpol η, 50 nM DNA; 3 nM hpol κ, 60 nM DNA; 50 nM hpol ι, 50 nM DNA; 10 nM Dpo4, 50 nM DNA; 100 nM Dpo1, 50 nM DNA; 50 nM hpol β, 50 nM DNA; 50 nM HIV-1 RT, 50 nM DNA. The percent activity was plotted as a function of the log of inhibitor concentration and fit to a four-parameter logistic model (equation 1) using Prism software (Graph Pad, San Diego, Calif.):

$$y = bottom + \frac{( top - bottom )}{1 + {( x / {IC}_{50} )}^{slope}} \qquad Eq . 1$$

The experiments were performed in triplicate and the mean (±standard deviation) of the IC₅₀ values calculated for each data set is reported.

Steady-State Kinetic Analysis of DNA Polymerase Activity.

The steady-state kinetic parameters defining polymerase activity in the presence of inhibitor were determined using the fluorescence-based reporter assay. Hpol q activity was monitored in the presence of increasing concentrations of dTTP (1, 5, 10, 20, 30, 50, 75, and 100 μM). The measured relative fluorescence units (RFUs) were converted to a nanomolar quantity by calculating the total change in fluorescence observed between the start of the reaction and the time point at which the fluorescence change was maximal, and considering that change to be 100% of substrate converted to product. The percentage of substrate converted to product was multiplied by the concentration of dsDNA in the reaction mixture. Product formation was then plotted as a function of time, and by considering only the linear portion of each curve, velocities were calculated for each dTTP concentration. These were then plotted as a function of dTTP concentration, and fit to a hyperbola. After correcting for enzyme concentration, the steady-state kinetic parameters were obtained as described previously. The experiments were then repeated in the presence of inhibitor (10, 20 and 40 μM) to determine the effect of the small-molecule upon Michaelis-Menten kinetics.

### Example 1

### Identification of Small-Molecule Inhibitors of Hpol η

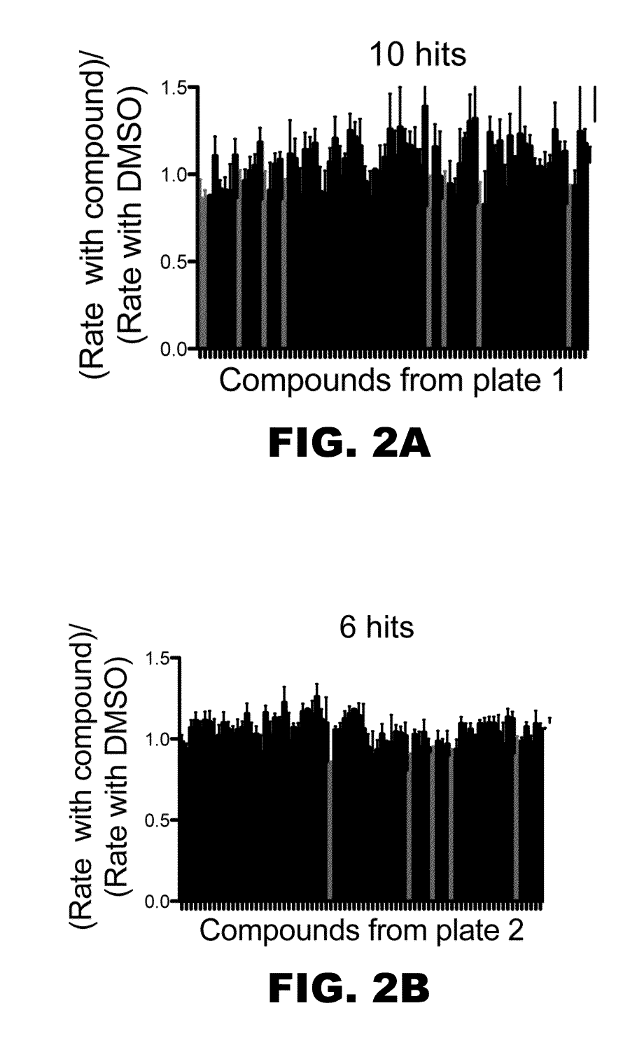

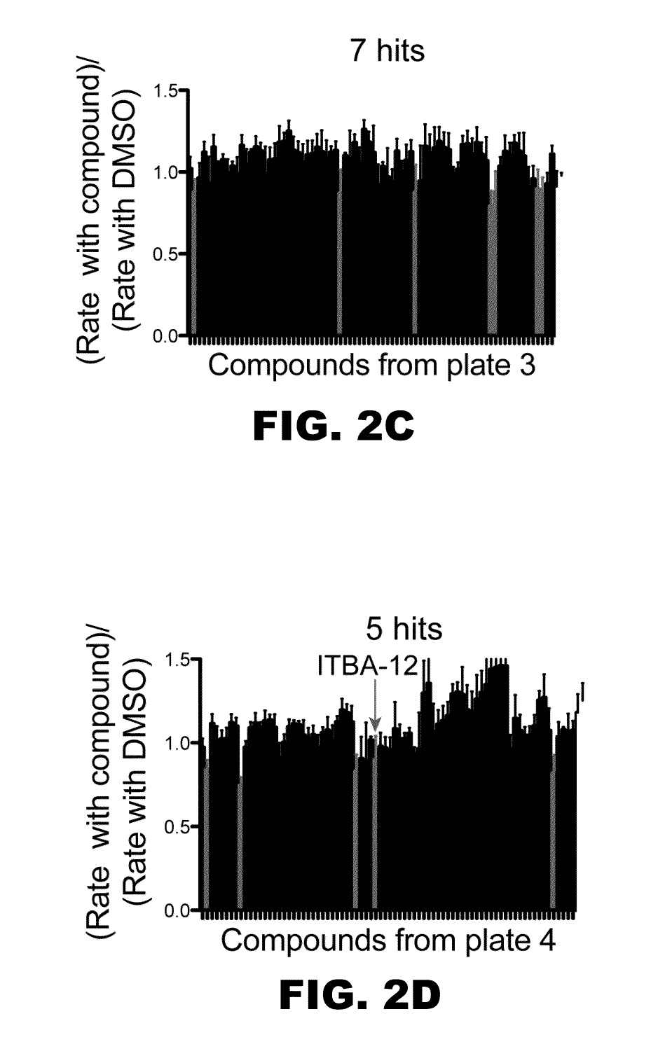

A small library of some 320 compounds was initially screened using a robust and quantitative assay that measures polymerase activity over time (FIG. 1A). The assay has been validated as a means of identifying small-molecule inhibitors of DNA polymerases of the Y-family of DNA polymerases and DNA polymerases of other DNA polymerase families (Yamanaka et al., 2012 PLoS One 7:e45032 and Dorjsuren et al., 2009, Nucleic Acids Res. 37:e128). The assay relies upon polymerase-catalyzed displacement of a fluorescently-labeled oligonucleotide and is reproducible (FIG. 1B). The initial screen to identify inhibitors of hpol n was performed with a final concentration of 6 μM compound. The experiments were performed in triplicate. The means and standard deviation for polymerase activity from all samples were calculated for each plate and compounds exhibiting a decrease in activity of greater than one standard deviation from the control experiment were considered as possible inhibitors (FIG. 2).

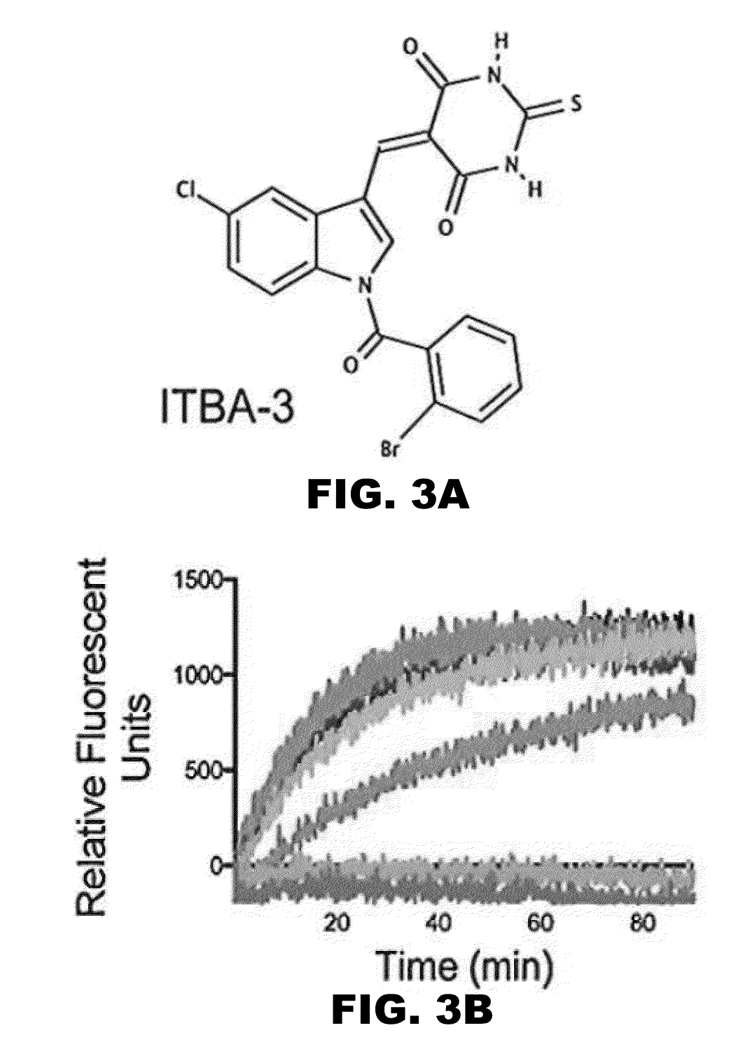

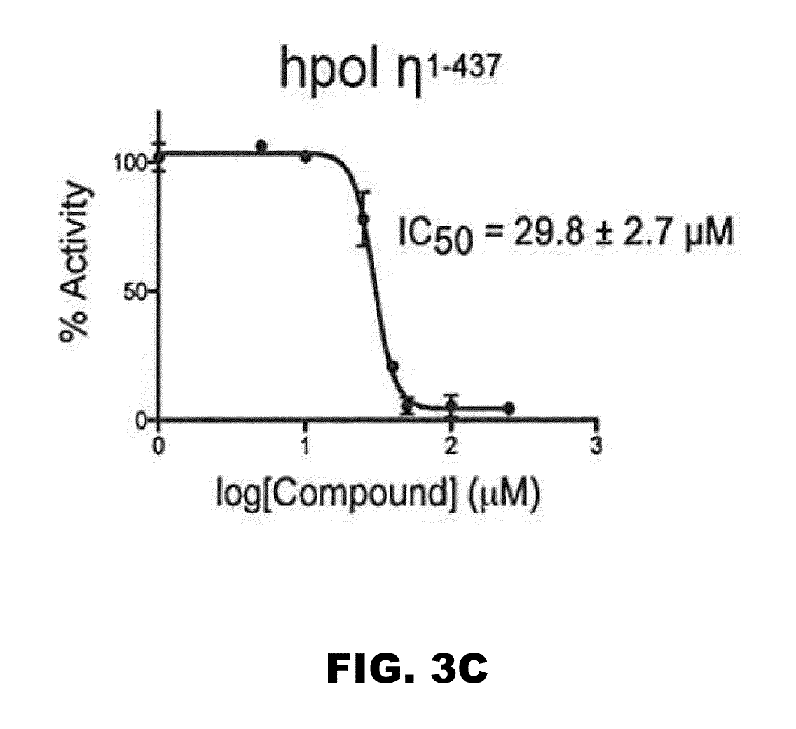

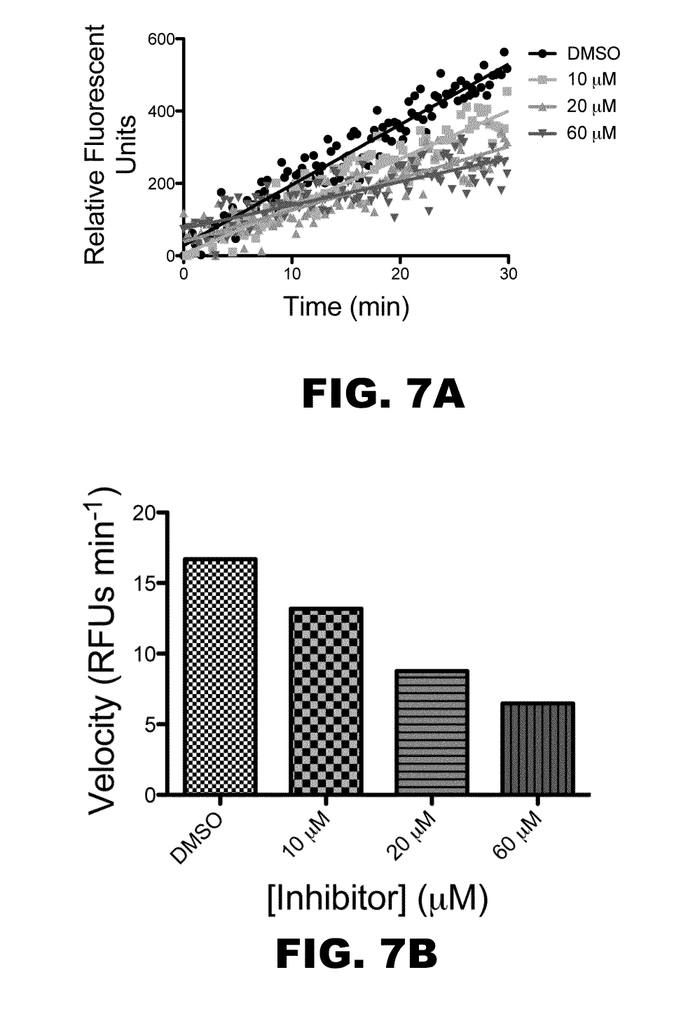

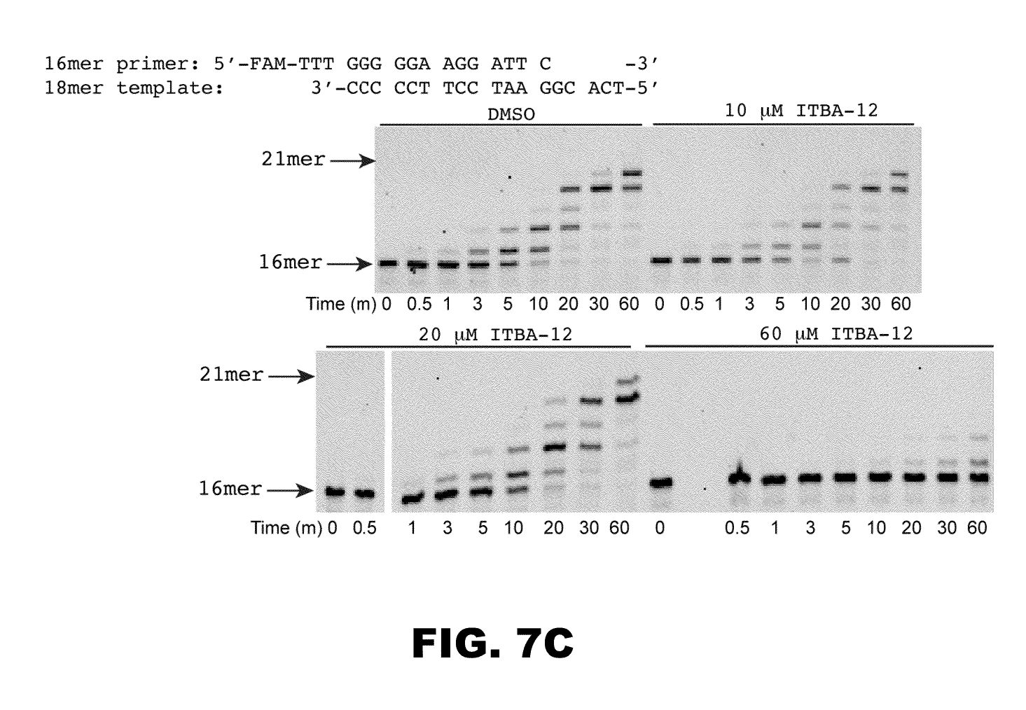

From this set of experiments, 28 potential polymerase inhibitors were identified. The success rate (˜9% of the compounds tested were found to inhibit polymerization) can be attributed in part to the targeted nature of the compound library. One of the compounds identified in the initial screen was (5-((1-(2-bromobenzoyl)-5-chloro-1H-indol-3-yl)methylene)-2-thioxodihydropyimidine-4,6-(1H,5H)-dione, which is an indole thio-barbituric acid (ITBA) derivative (ITBA-3, FIG. 3A). ITBA-3 was re-tested for polymerase inhibition by monitoring polymerase activity with the fluorescence assay at increasing concentrations of inhibitor (6 μM, 13 μM and 20 μM). A dose-dependent decrease in polymerase activity was observed using both fluorescence and gel-based analyses (FIG. 7) A more rigorous determination of the IC₅₀ value for ITBA-3 mediated inhibition of hpol q was performed (FIG. 3B). The measured IC₅₀ value for ITBA-3 was found to be 29.8±2.7 μM (FIG. 3C). From these results, it was determined that ITBA-3 is a reasonable starting point for the development of novel polymerase inhibitors.

### Example 2

### Determination of the In Vitro Specificity of ITBA-3 Against Different DNA Polymerases

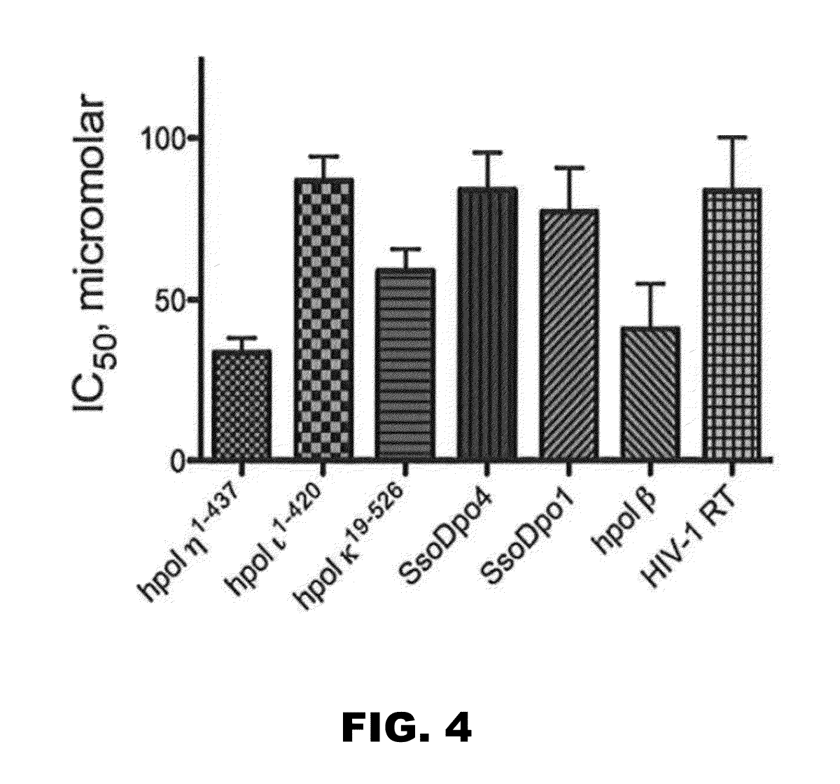

In order to determine the specificity of ITBA-3 against the Y-family member hpol η, the IC₅₀ values for inhibition of six other polymerases were measured (FIG. 4). It was found that hpol η exhibited the most potent inhibition by ITBA-3 when compared with the other polymerases tested. Of the other Y-family polymerases tested, only hpol κ showed an IC₅₀ value that was noticeably reduced relative to hpol ι and Dpo4 from Sulfolobus solfataricus. However, the IC₅₀ value for ITBA-3 inhibition of hpol κ is twice as high as that measured for hpol η, suggesting some discrimination between the Y-family enzymes tested here. Next, the model B-family polymerase Dpo1 from S. solfataricus was also tested for inhibition by ITBA-3, and the IC₅₀ value was determined to be near 80 μM. A similar value was observed for HIV-1 RT. Besides hpol η IC₅₀, only hpol β showed an IC₅₀ value below 50 μM, which is interesting since expression of this enzyme also appears to modulate the toxicity of drugs like cisplatin. Based on these results, it was concluded that ITBA-3 exhibits modest selectivity against hpol η.

### Example 3

### Mechanism of Hpol η Inhibition by ITBA-3

Next, the mechanism of polymerase inhibition by ITBA-3 was investigated. The Michaelis-Menten kinetic parameters describing hpol η activity were measured in the presence of increasing concentrations of inhibitor. By varying the concentration of dTTP in the reaction mixture, the turnover number (k_{cat}) and Michaelis constant (K_{m,dTTP}) for hpol η were determined in the absence of inhibitor and at three concentrations of ITBA-3 (Table 3). Increasing the amount of inhibitor in the reaction mixture clearly results in an increase in the Michealis constant but does not appear to affect the turnover number. These results are indicative of a competitive mode of inhibition by ITBA-3.

TABLE 3
Steady-state kinetic parameters for hpol q activity in the
presence of ITBA-3 and varying concentrations of dTTP.

| dTTP (μM) | kcat (min⁻¹) | Km, dTTP (μM) |
|---|---|---|
| — | 2.5 ± 0.2 | 6.5 ± 2.0 |
| 10 | 2.4 ± 0.1 | 6.0 ± 1.2 |
| 20 | 3.0 ± 0.3 | 18.3 ± 9.2 |
| 40 | 3.1 ± 0.2 | 19.8 ± 6.5 |

### Example 4

### Structure-Activity Relationships for Inhibition of Hpol η by ITBA Derivatives

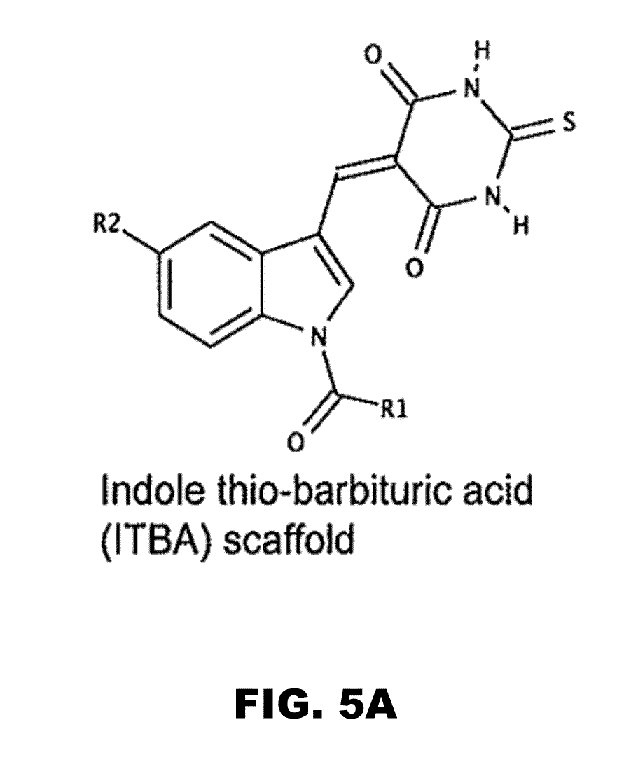

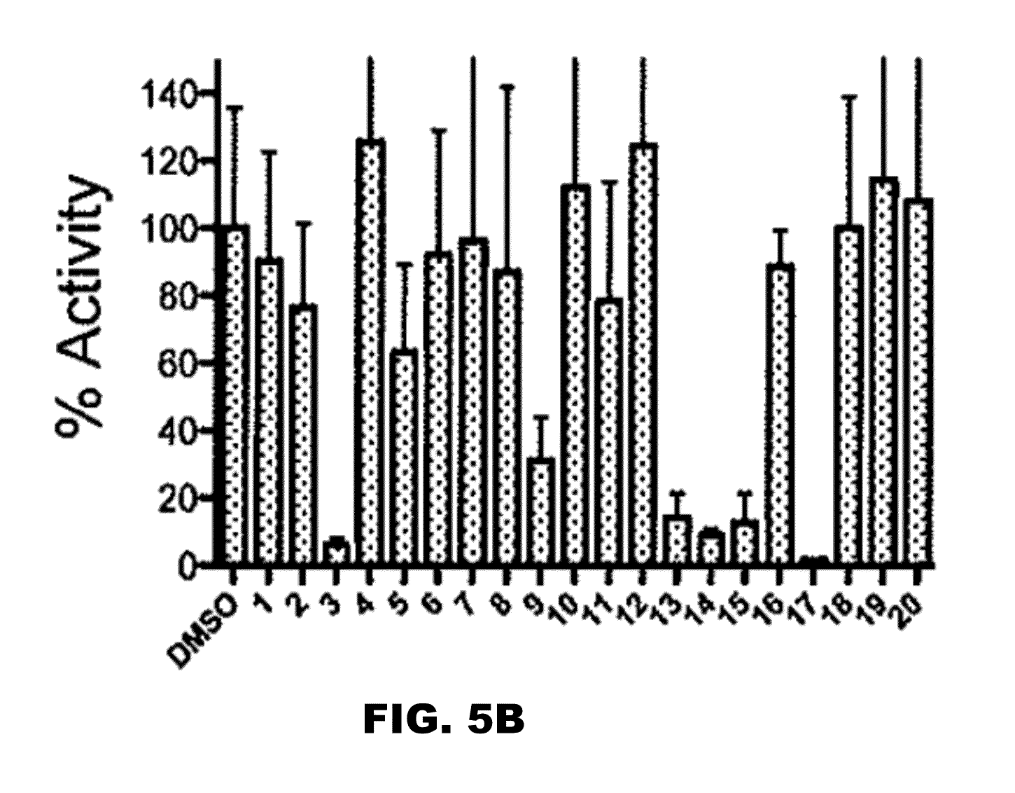

A series of ITBA compound 3 derivatives were prepared as described elsewhere in the materials and methods above. In total, 20 compounds derived from the ITBA scaffold shown FIG. 5A were tested for their ability to inhibit hpol η. R¹ and R² groups are as described in Table 4

TABLE 4
ITBA derivatives tested for structure-
activity relationship against hpol q

| Compound | R1 | R2 |
|---|---|---|
| 1 | phenyl | — |
| 2 | 2-bromophenyl | — |
| 3 | 2-bromophenyl | Cl |
| 4 | — | — |
| 5 | phenyl | Cl |
| 6 | — | — |
| 7 | phenyl | Br |
| 8 | — | — |
| 9 | 4-methoxy-phenyl | Cl |
| 10 | 4-methoxy-phenyl | Br |
| 11 | 4-methoxy-phenyl | methoxy |
| 12 | 4-fluoro-phenyl | — |
| 13 | 2-naphthyl | — |
| 14 | 2-naphthyl | Cl |
| 15 | 2-naphthyl | methoxy |
| 16 | 1-naphthyl | — |
| 17 | 1-naphthyl | Cl |
| 18 | 1-naphthyl | methoxy |
| 19 | 4-CN—C₆H₄ | — |
| 20 | 4-COOCH₃—C₆H₄ | — |

The 20 compounds were tested at a concentration of 50 μM and the percent activity relative to the control assay was plotted (FIG. 5B). The parent compound (ITBA-1) shows almost no inhibitory action at 50 μM. The addition of a bromine at the 2 position of the R¹ phenyl ring (ITBA-2) somewhat improves the inhibition of hpol η, but it is the addition of a chlorine atom at position 5 on the indole ring (R² of ITBA compound 3) that causes a dramatic improvement in activity against the polymerase (FIG. 5B, compound 3).

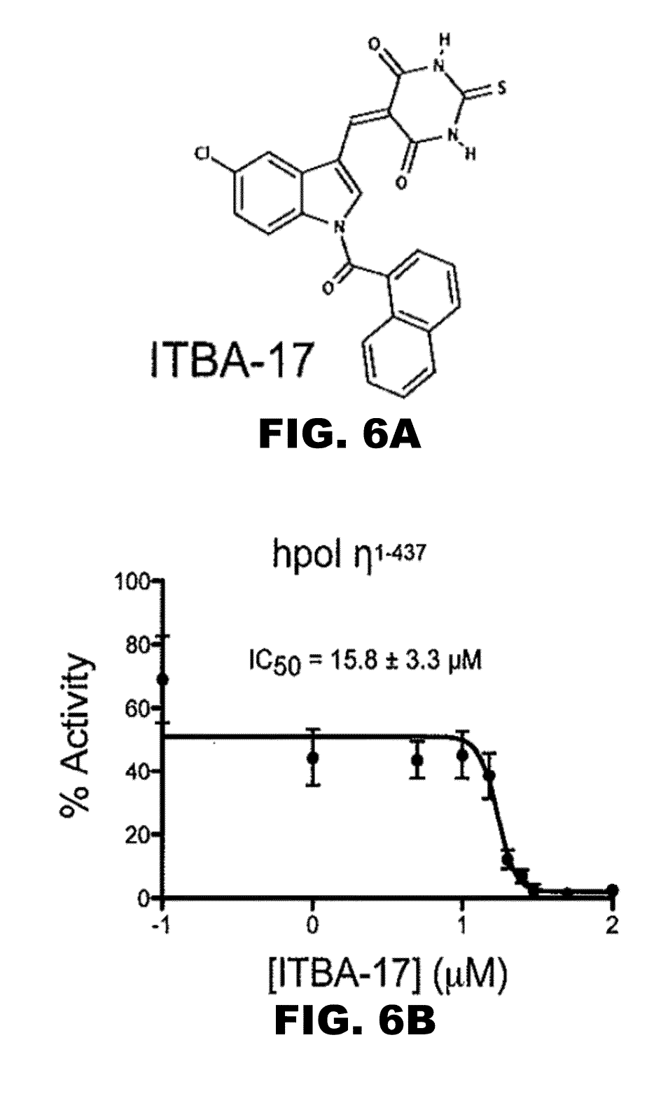

Removing the bromine from position 2 of the R¹ substituent attenuates the activity of compound 5, suggesting that the chlorine atom alone is not responsible for the increased potency of compound 3 (ITBA-3). However, flexibility in the identity of the R¹ substituent is indicated by compound 9 (ITBA-9), which has a 4-methoxy-substituted phenyl ring. Furthermore, the most potent ITBA derivatives prepared in our study possessed a naphthyl moiety as the R¹ substituent. Both 1-naphthyl and 2-naphthyl-substituted ITBA derivatives were compared. The three ITBA derivatives with a 2-naphthyl substituted moiety at the R¹ position each show roughly equal activity against hpol η (FIG. 5 compares results for ITBA compounds 13, 14 and 15). Substitution of chlorine at the 5 position of the indole ring (14) does show the greatest inhibitory action, but it is not markedly different from the unsubstituted R² position (13) or the methoxy substituted molecule (15). In contrast to these results, the 1-naphthyl substituted ITBA molecules (16, 17 and 18) display huge differences in the observed level of polymerase inhibition. Compound 17 was found to be the most potent inhibitor of hpol η activity identified in our study. The measured IC₅₀ value for ITBA compound 17 inhibition of hpol η was found to be 15.8±3.3 μM (FIG. 6), which is about half the value measured for ITBA compound 3.

### Conclusion for Examples 1-4

A library of novel compounds was screened to identify potential small molecule inhibitors of translesion DNA polymerases, such as hpol η. Of the 28 leads identified in the screen, ITBA-3 was determined early on to be a true polymerase inhibitor, as assessed by complementary assays (FIG. 7). ITBA-3 exhibits the most potency against hpol η, with a comparable IC₅₀ value for inhibition of another Y-family member, hpol κ and the X-family polymerase, hpol β. The mechanism of inhibition by ITBA-3 was probed by both steady-state kinetic analysis and by chemical modification of the ITBA scaffold. The competitive mode of inhibition suggests that ITBA may interfere with some aspect of dNTP binding. The top hits from in silico docking results with SwissDOCK localize ITBA-3 and ITBA17 to a pocket between the finger and little finger domains (data not shown). This pocket is also picked up when another small-molecule inhibitor of Y-family members, candesartan cilexitil, was docked using SwissDOCK. Additionally, a second small-molecule binding pocket was identified in the top 10 docking hits for the ITBA compounds, and the second pocket lies near the finger domain of Y-family polymerases (data not shown). Notably, the finger domain possesses residues that are crucial for stabilization of the incoming dNTP within the active site of all DNA polymerases, though the secondary structures defining the “finger” domain vary between polymerase families. It is possible that the ITBA molecules bind to both pockets. Alternatively, recent crystal structures with hpol n bound to cisplatin-modified DNA reported the identification of a second nucleotide binding site near Trp²⁹⁷, when crystals were soaked with high concentrations of dATP (>0.5 mM). The hydrophobic pocket identified in the crystal structure is located near the thumb domain of the protein and could interfere with conformational changes identified in this region for other Y-family members. Further structural characterization of ITBA-mediated inhibition of hpol η may be performed.

In addition to identifying a small molecule inhibitor of hpol η, structure-activity relationships were used to improve the potency of the ITBA compound initially identified. The presence of a chlorine atom at position 5 of the indole ring of ITBA appears to be necessary but not sufficient to impart the maximum inhibitory effect observed. The comparative improvement on polymerase inhibition by adding a naphthyl group at the R¹ substituent is also interesting. While the 2-naphthyl group appears to tolerate substitutions at the R² position without too much effect on activity (FIG. 5 compares ITBA compounds 13, 14 and 15), the 1-naphthyl R¹ substituent appears to be highly dependent upon the 5-chloro substitution on the indole ring (FIG. 5 compares ITBA compound 16, 17 and 18).

Further modification of the ITBA scaffold may improve the potency and specificity of the class of polymerase inhibitors identified herein. Experiments to determine whether these compounds can modulate cell survival in the face of DNA damaging agents may be performed.

### Introduction for Examples 5-9

Any type of cell death is characterized by nuclear DNA fragmentation, which is a limiting and necessary mechanism of cell death. After fragmentation of DNA, cell death becomes irreversible. DNA fragmentation is catalyzed by a group of enzymes called “apoptotic endonucleases.” One of the most active representatives of this group is Endonuclease G (EndoG), a nuclear DNA-coded mitochondrial enzyme that relocates to the nucleus and fragments DNA during apoptosis. EndoG has unique site-selectivity from which the enzyme acquired its name; EndoG initially attacks poly(dG).poly(dC) sequences in double-stranded DNA.

Genetic inactivation of EndoG (in knockout animals or cells) provides protection against various injuries. No specific inhibitors of EndoG have been identified in mammals; however Drosophila has a specific protein inhibitor of EndoG called EndoGI. The therapeutic value of this protein inhibitor is insignificant because it is expressed only in Drosophila, and because it is a protein, making its administration problematic. Inhibition of EndoG expression by siRNA for research purposes has been described.

Currently, there are no pharmaceutically viable chemical inhibitors of EndoG. Such inhibitors would be useful for protection of normal tissues from various injuries, including chemical/drug poisoning, hypoxia, or physical injury. The same inhibitors may also be applicable for promotion of cell death in cancer cells by increasing resistance of normal tissues surrounding tumors. The Examples presented herein describe the design and synthesis of a series of indomethacin analog small molecule inhibitors of EndoG. The activity of the compounds against EndoG in cells is also described.

### Example 5

### Screening for EndoG Inhibitors

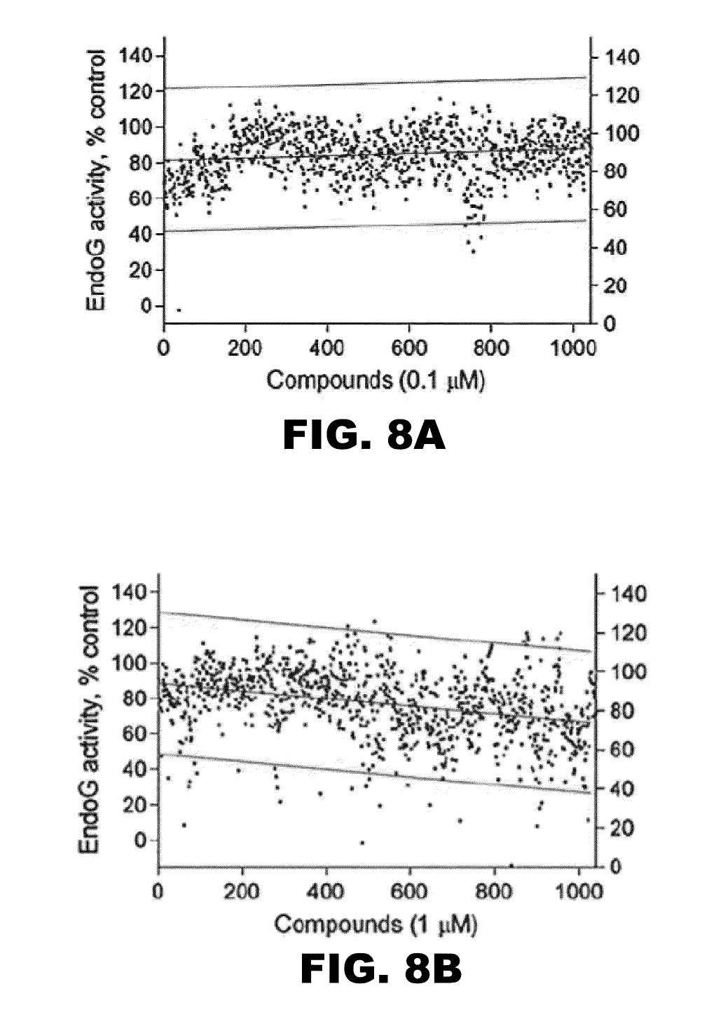

A chemical library containing 1,040 chemical compounds was prepared and screened utilizing a high throughput assay. Two different concentrations of test compounds (0.1 and 1 μM) were used in the assay to identify inhibitors. Compounds that showed 40% of control EndoG activity at the two compound concentrations were chosen for further analysis. The percent activity was determined from the middle trend line of control EndoG activity for all compounds tested. As expected, the lower concentration of compounds used in the screen (FIG. 8A) generated a smaller number of active compounds than the higher concentration of compounds (FIG. 8B).

### Example 6

### Confirmation of Anti-EndoG Activity

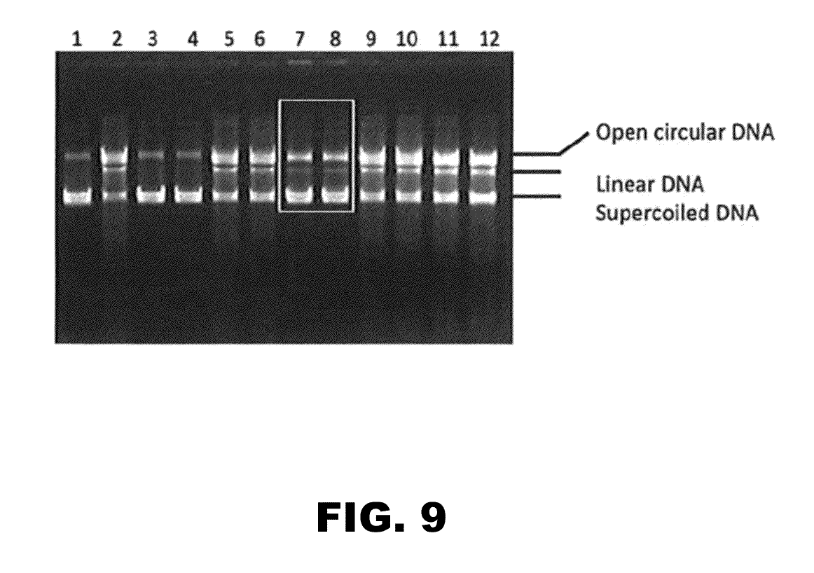

Anti-EndoG activity of compounds identified in the high throughput screen in Example 5 was confirmed using a plasmid incision assay (PIA) (FIG. 9). Using this assay, two potential EndoG inhibitors, PNR-3-80 and PNR-3-82 (lanes 7 and 8), significantly protected the plasmid DNA from degradation by EndoG. In the presence of the potential EndoG inhibitors (lanes 7 and 8), most of the supercoiled plasmid DNA remained uncleaved compared to the negative control (lane 2), and other candidate compounds (lanes 5, 6, 9, 10, 11, and 12).

### Example 7

### Determination of IC₅₀ Values of the EndoG Inhibitors

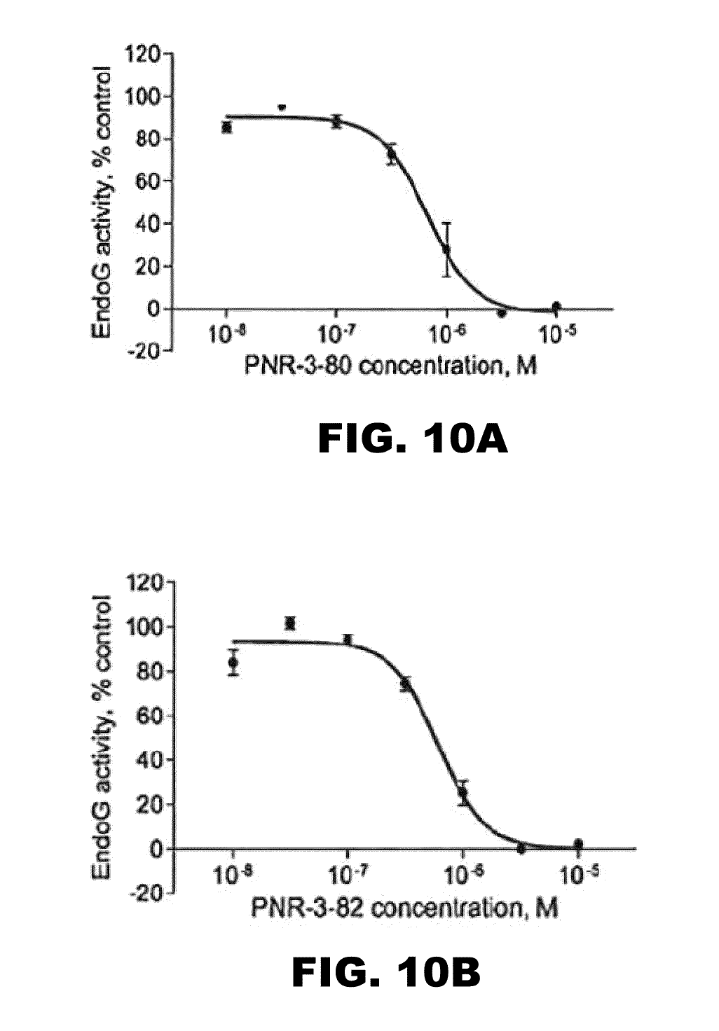

In order to determine the IC₅₀ of each anti-EndoG candidate compound of Example 6, the EndoG screening assay was repeated with increasing concentrations of inhibitor, and the IC₅₀ was determined. The IC₅₀ values of PNR-3-80 (0.671 μM) and PNR-3-82 (0.613 μM) were determined utilizing the EndoG screening assay (FIGS. 10A and 10B).

### Example 8

### Specificity of EndoG Inhibitors to Apotpotic Endonucleases

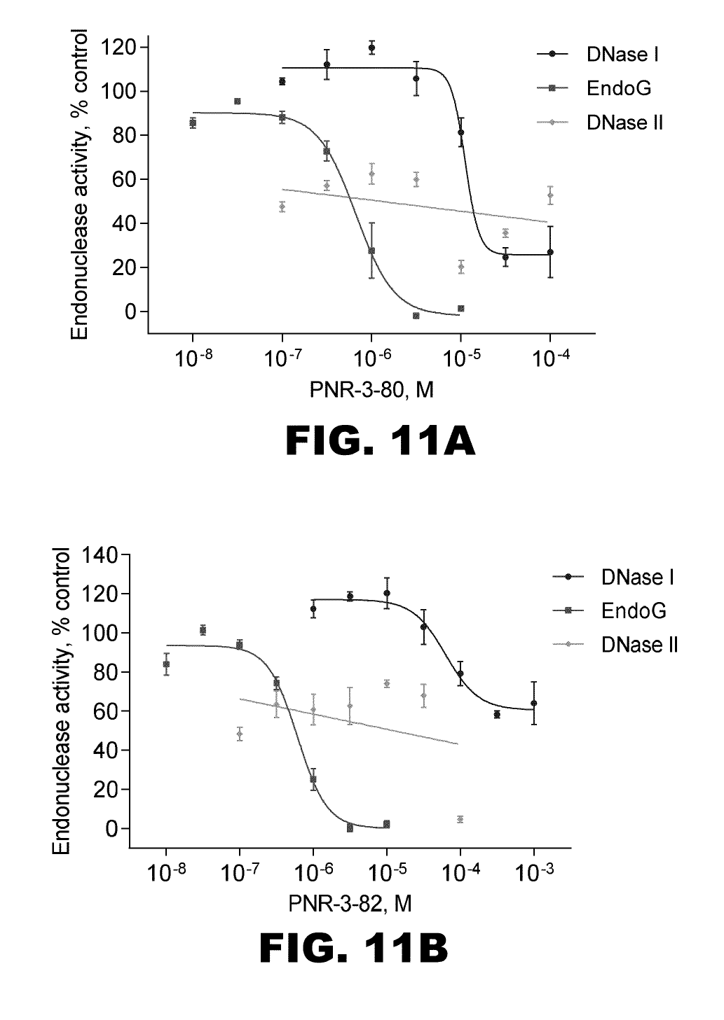

Specificity of a potential inhibitor is important for any in vitro or in vivo application, and generally defines the usefulness of an inhibitor. To determine specificity of the inhibitors for the two EndoG inhibitors, PNR-3-80 and PNR-3-82, their IC₅₀ values for EndoG were compared using the screening assay with the IC₅₀ values for two other endonucleases, DNase I and DNAse II (FIG. 11). These endonucleases were chosen because they represent the majority of endonuclease activity in most mammalian cells and because of their availability as pure enzymes. The data obtained showed that PNR-3-80 and PNR-3-82 were ˜17 and 104, respectively, times more specific to EndoG than DNase I. The inhibitors did not have any effect on DNase II activity.

The IC₅₀ value of PNR-3-80 against EndoG was about 17 times lower than the IC₅₀ value of the compound against DNaseI, whereas The IC₅₀ value of PNR-3-82 against EndoG was more than 100 times lower than the IC₅₀ value of the compound against DNase I (Table 5). The IC50 values of these compounds was also tested against DNase II as shown in FIG. 11.

TABLE 5

| | PNR-3-80 | PNR-3-82 |
|---|---|---|
| DNase I | 11.21 μM | 63.31 μM |
| EndoG | 0.67 μM | 0.61 μM |

### Example 9

### The EndoG Inhibitors Act as Modulators of Alternative Splicing and Transcriptional Regulators of DNase I Expression

To determine if inhibiting EndoG activity affects alternative splicing of nucleic acids encoding DNase I, ZR-75-1 human breast cancer cells were incubated with the two EndoG inhibitors described above for 24 hours. EndoG expression, as well as expression of two alternatively-spliced DNase I mRNAs, were measured by real-time RT-PCR. The alternatively-spliced DNase I mRNAs assayed in this example encode an active full-size mature DNase I, and the Δ4DNase I isoform. The Δ4DNase I isoform does not exhibit enzymatic endonuclease activity, but instead is a dominant-negative suppressor of the active DNase I.

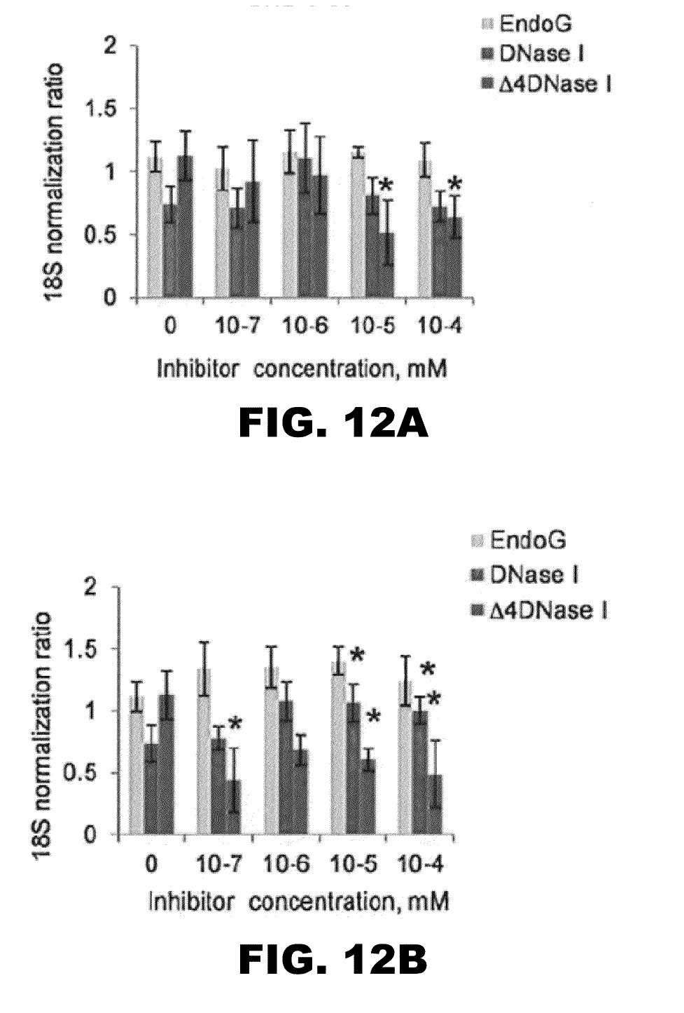

The RT-PCR results showed that EndoG inhibitors regulate alternative splicing of DNase I (FIG. 12). In the absence of EndoG inhibitor, RT-PCR results showed that both the full length and Δ4 isoforms of DNase I assayed were expressed in the cell. However, the higher expression of the Δ4DNase I isoform compared to the expression of the active DNase I isoform indicates that these cells expressed active DNase I, but that the activity of the active DNase I isoform may have been suppressed by the higher expression of the Δ4DNase I isoform. Expression of DNase I isoforms was reversed in cells treated with EndoG inhibitors. When cells are treated with EndoG inhibitors, nucleic acid sequences expressing active DNase I were expressed at a higher level than nucleic acid sequences encoding the Δ4DNase I isoform. The expression of EndoG was not affected.

### Conclusion for Examples 5-9

From the library of substituted N-naphthoylindolethiobarbituric acid analogs, two analogs were identified as inhibitors of EndoG. PNR-3-80 showed the most potency against EndoG with an IC₅₀ value of 0.671 μM, and another structurally related compound, PNR-3-82 exhibited an IC₅₀ value of 0.613 μM against EndoG. These compounds represent the first examples of small molecule indole analogs with EndoG inhibitory activity, and are regarded as important novel leads for the development of more potent and selective agents with therapeutic potential. An additional and potentially therapeutically important feature of the new inhibitors is that they regulate alternative splicing and overall expression of active DNase I.

### Experimental Procedure for Examples 5-9

### Compounds

A series of substituted N-alkyl and N-aroyl-1H-indol-3-yl)methylene)-barbiturates or 2-thiobarbiturates indomethacin analogs were synthesized using a two-step synthesis process as described in Reaction Scheme 2. In step (a), aromatic substituted N-benzoylindole-3-carboxaldehydes (2a-z) were synthesized in 85-90% yield by treating the appropriately substituted indole-3-carboxaldehyde (1a-d) with various substituted benzoyl halides under phase-transfer catalytic (PTC) conditions utilizing triethylbenzyl ammonium chloride (TEBA) and a mixture of dichloromethane in 50% w/v aqueous NaOH solution. A series of substituted N-alkyl and N-aroyl-1H-indol-3-yl)methylene)-barbiturates or 2-thiobarbiturates indomethacin analogs (3a-z) were synthesized by aldol condensation of the appropriate N-substituted 2-methyl indole-3-carboxaldehyde with either barbituric acid or thiobarbituric acid and its related compounds. (Reaction Scheme 2 and Table 6). The indole-3-aldehyde and barbituric acid or thiobarbituric acid are stirred in methanol at room temperature for about 4-6 hours. The structure and purity of these derivatives was verified by ¹H and ¹³C-NMR spectroscopy.

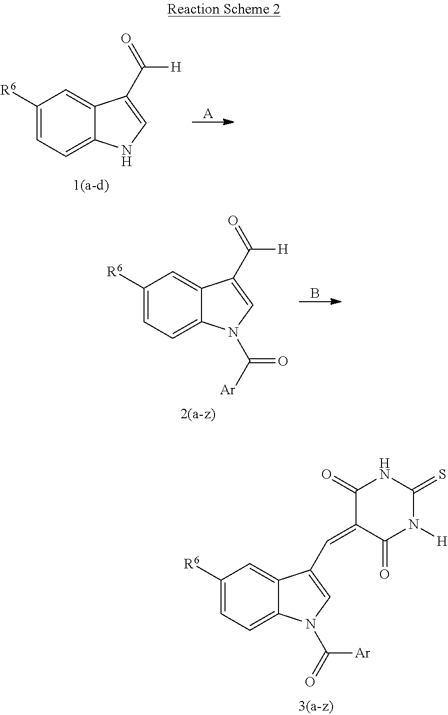

TABLE 6

| Structure No. | R⁶ | Ar |
|---|---|---|
| A | H | C₆H₅ |
| B | Cl | C₆H₅ |
| C | Br | C₆H₅ |
| D | OCH₃ | C₆H₅ |
| E | H | 4-F—C₆H₄ |
| F | Cl | 4-F—C₆H₄ |
| G | Br | 4-F—C₆H₄ |
| H | H | 4-OCH₃—C₆H₄ |
| I | Cl | 4-OCH₃—C₆H₄ |
| J | Br | 4-OCH₃—C₆H₄ |
| K | OCH₃ | 4-OCH₃—C₆H₄ |
| L | H | 4-CN—C₆H₄ |
| M | Cl | 4-CN—C₆H₄ |
| N | H | 4-COOCH₃—C₆H₄ |
| O | Cl | 4-COOCH₃—C₆H₄ |
| P | H | 2-Br—C₆H₄ |
| Q | Cl | 2-Br—C₆H₄ |
| R | Br | 2-Br—C₆H₄ |
| S | OCH₃ | 2-Br—C₆H₄ |
| T | H | 1-naphthyl |
| U | Cl | 1-naphthyl |
| V | Br | 1-naphthyl |
| W | OCH₃ | 1-naphthyl |
| X | H | 2-naphthyl |
| Y | Cl | 2-naphthyl |
| Z | OCH₃ | 2-naphthyl |

About 1,040 compounds were synthesized. All compounds had a MW of about 500 Da. Each compound was dissolved in DMSO in 96-well plates (Thermo, Rochester, N.Y.) to generate a 10 mM solution.

### High-Throughput EndoG Screening Assay

A reaction mixture was prepared in each well of a white 96-well plate (Costar, Corning, N.Y.) as follows: 0.25 μM Cy5.5-labeled oligonucleotide (described in US provisional patent filed Oct. 19, 2012, Ser. No. 61/716,097) 0.3 mM MgCl₂, 10 mM Tris-HCl, pH 7.4, 1 μl DMSO containing 5 or 50 ng of test compound, and nuclease-free water for a total reaction volume of 100 μl. The background (negative control) and uninhibited EndoG samples were measured using DMSO only, or DMSO containing recombinant EndoG (4 μg/ml), respectively. After addition of EndoG, fluorescence intensity was kinetically measured on a Bio-Tek Synergy 4.0 plate reader at 37° C. and mean velocity (mRFU/min) within 20 min was automatically calculated by the plate reader. The background was subtracted prior to the calculation of EndoG activity (%). The percent of EndoG activity was calculated from the mean velocity of a compound divided by the mean velocity of DMSO with recombinant EndoG, and the obtained value multiplied by 100.

### Plasmid Incision Assay (NA)

A reaction mixture was prepared containing 0.5 μg pECFP plasmid DNA, 2 mM CaCl₂, 5 mM MgCl₂, 10 mM Tris-HCl, pH 7.4, 0.5 mM dithiothreitol. The test compound [50 ng in DMSO (1 μl)] was added. Recombinant EndoG was then added to the final concentration of 200 ng/ml and the reaction was incubated for 1 h at 37° C. The reaction was terminated by adding 2 μl of 10 mM Tris-HCl, pH 7.4, 1% SDS, 25 mM EDTA, 7.2 mM bromophenol blue. The samples were run in 1% agarose gel in Tris-acetate-EDTA buffer (40 mM Tris, 20 mM acetic acid, 1 mM EDTA, pH 8), at 7 V/cm for 35 min, and DNA was stained with ethidium bromide. An EagleEye scanning densitometer (Stratagene, La Jolla, Calif.) was utilized to quantify the relative amount of endonuclease-treated plasmid DNA present in a covalently-closed circular (supercoiled) DNA, open circular DNA, or linear DNA, or in a digested form.

### Example 10

### Screening to Identify Inhibitors of HCV NS3 Helicase

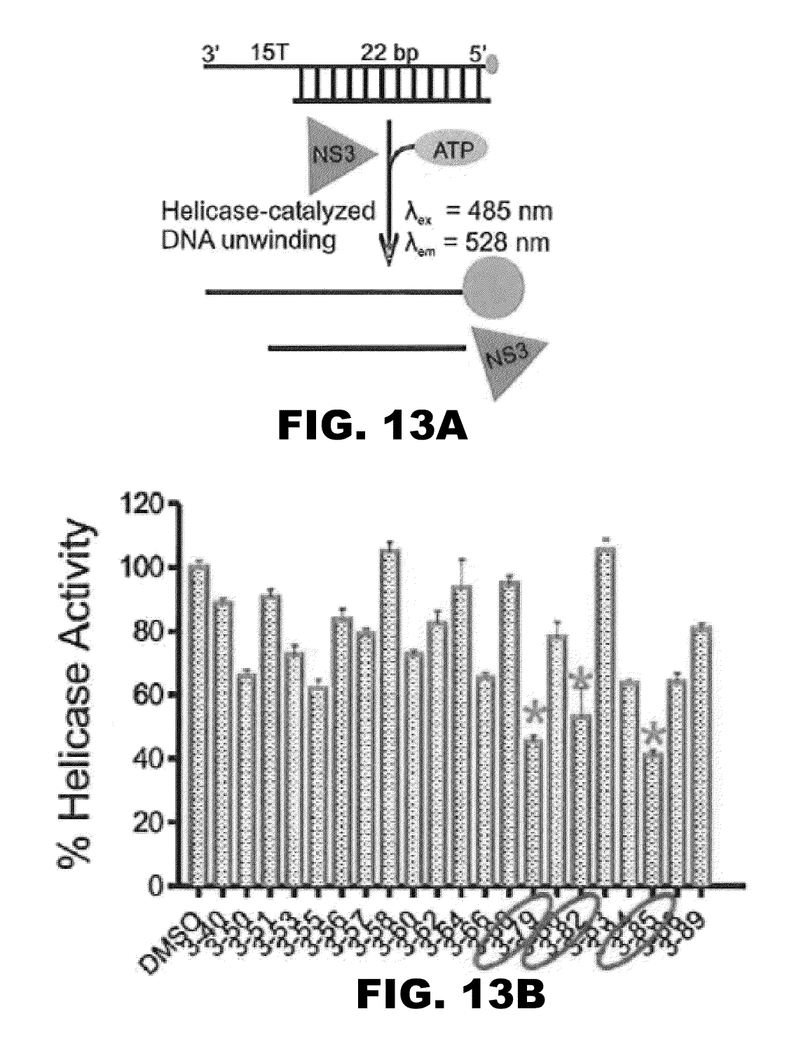

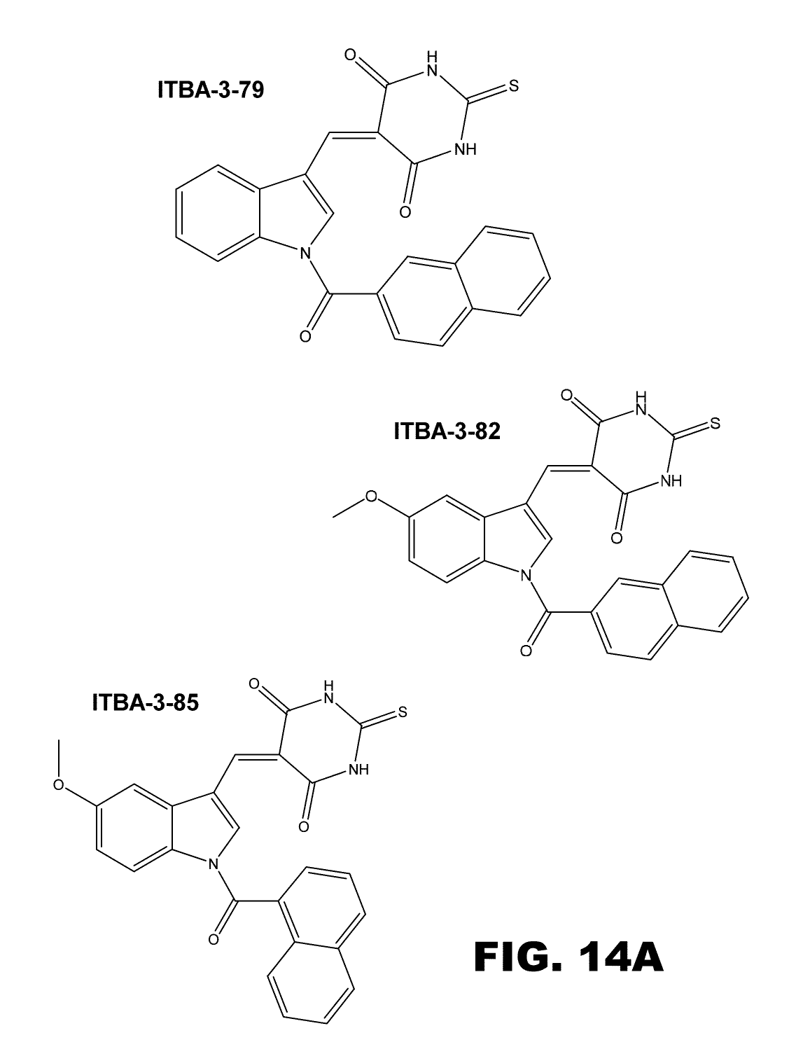

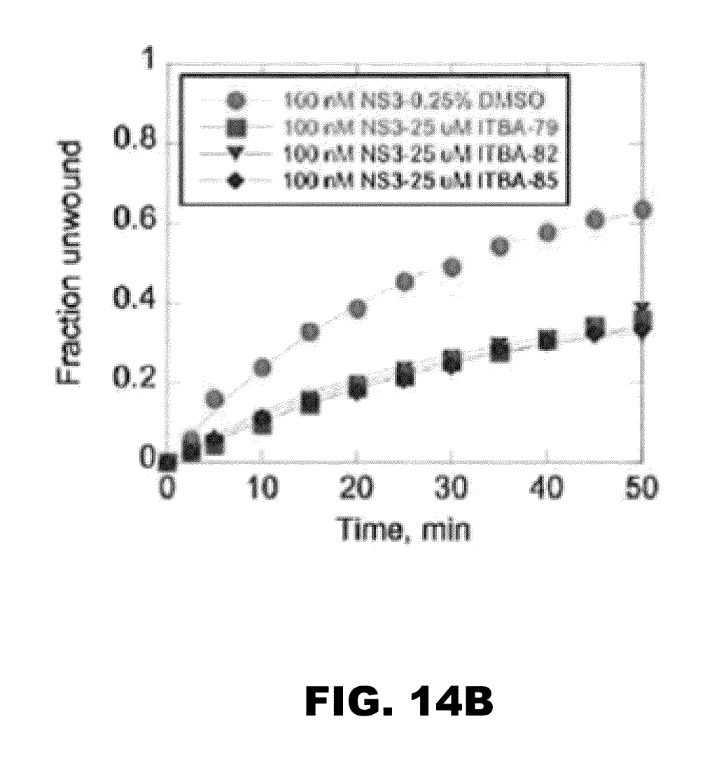

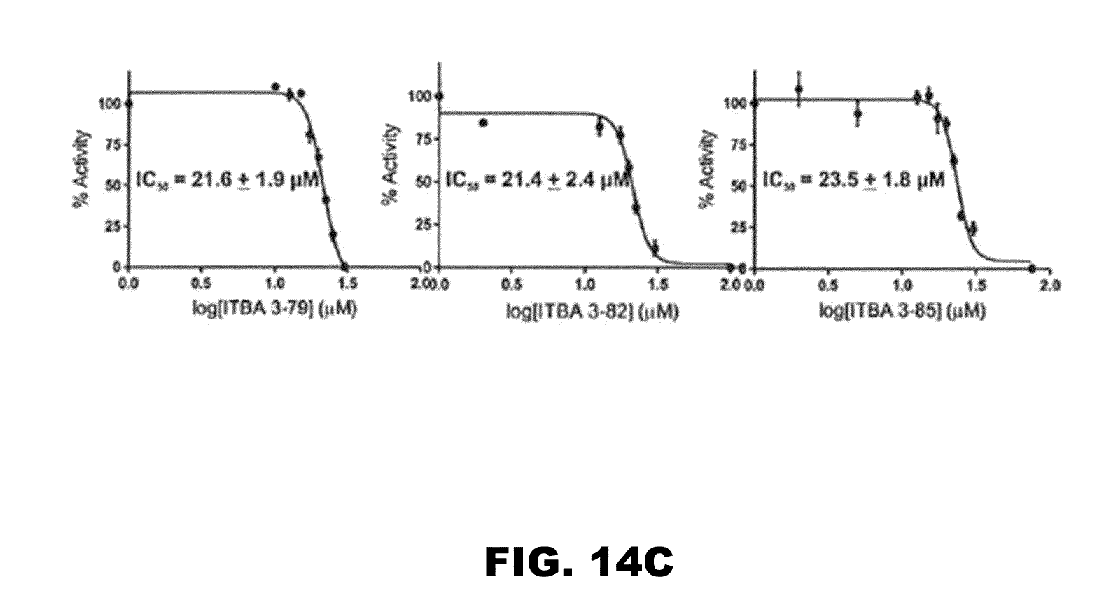

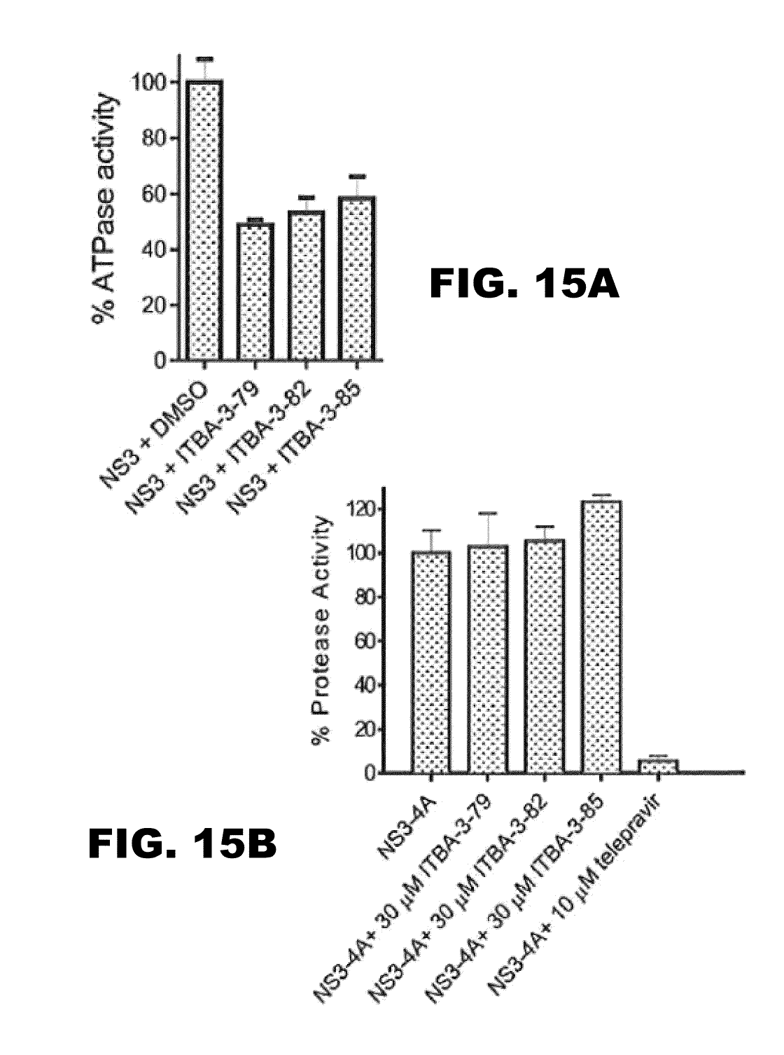

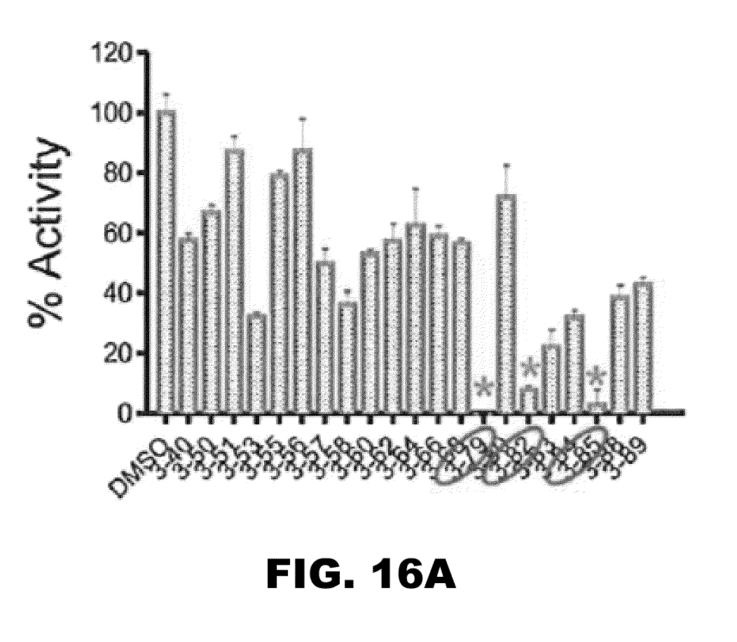

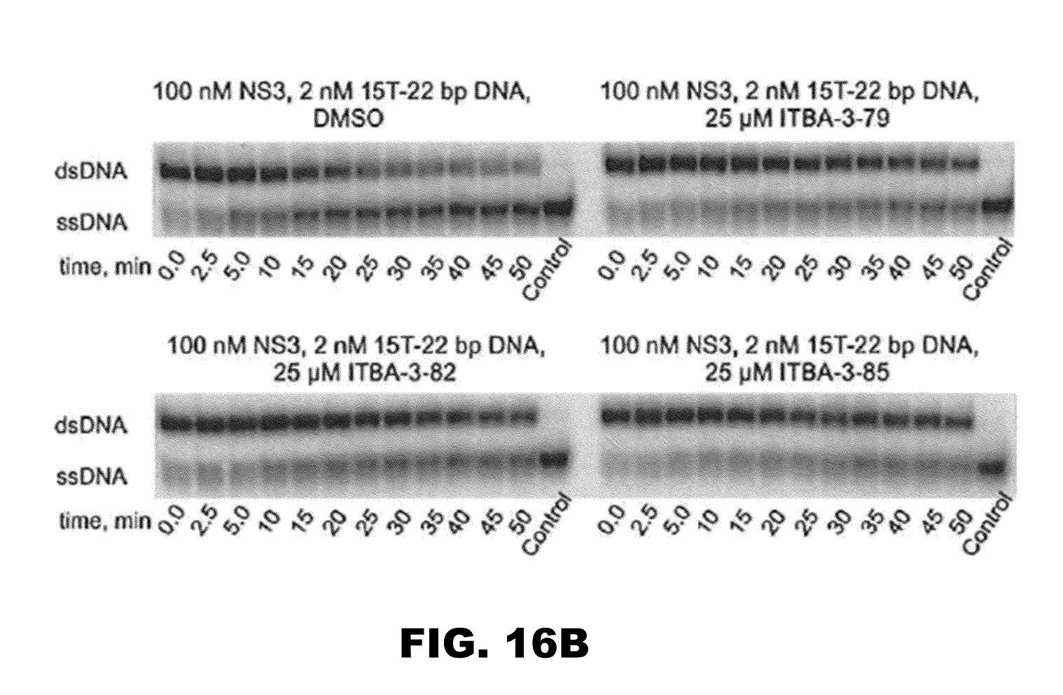

A library of compounds was screened using a previously validated quantitative assay that measures helicase activity over time. The assay relies upon helicase-catalyzed displacement of a fluorescently-labeled oligonucleotide (FIG. 13A). The concentration of enzyme and compounds used was 250 nM and 20 μM respectively. The experiments were performed in triplicate. The mean and standard deviation for helicase activity were calculated and compounds exhibiting a decrease in activity of greater than one standard deviation from the control experiment were considered as possible inhibitors (FIG. 13B). Three potential helicase inhibitors were identified using the fluorescence based assay. The fact that 15% of the compounds tested were found to inhibit HCV NS3 catalyzed duplex NA unwinding can be attributed to the targeted nature of the compound library. The inhibitor compounds identified in our screen were ITBA-3-79, ITBA-3-82 and ITBA-3-85 (FIG. 14A). We confirmed the inhibition of the helicase activity using the fluorescence assay at a higher concentration of inhibitors (30 μM) (FIG. 13A). A decrease in helicase activity was observed in the gel-based analysis (FIGS. 14B and 16B). The IC₅₀ value for ITBA-3-79, ITBA-3-82 and ITBA-3-85-mediated inhibition of NS3 helicase ranged˜20 μM (FIG. 14C). The ATPase activity of NS3 (50 μM) was analyzed in the presence of ITBA-3-79, ITBA-3-82 and ITBA-3-85 using a coupled spectrophotometric assay (Raney and Benkovic 1995, (FIG. 15). The protease activity of NS3 was analyzed in the presence of ITBA-3-79, ITBA-3-82 and ITBA-3-85 by using 50 nM NS3-4A and 100 nM substrate (Ac-Asp-Glu-Asp-EDANS-Glu-Glu-Abu-L-Lactoyl-Ser-Lys DABCYL-NH2, FIG. 15. The NS3 helicase activity was analyzed in the presence of 25 μM ITBA-3-79, ITBA-3-82 and ITBA-3-85 (FIG. 16).

### Conclusions Example 10

We identified a novel class of compounds that inhibit HCV NS3 helicase using a robust and quantitative fluorescence based helicase assay. Three indole thio-barbituric acid (ITBA) derivatives (ITBA-3-79, ITBA-3-82, and ITBA-3-85) were identified as inhibitors of the HCV NS3 helicase. The IC5c. values for ITBA-79, ITBA-82, and ITBA-85-mediated inhibition of NS3 helicase were 21.6±1.9 μM, 21.4±2.4 μM, and 23.5±1.8 μM respectively. The standard helicase assay using gel electrophoresis confirmed the inhibition of NS3 helicase activity by these compounds. These compounds do not block protease activity and their mechanism of inhibition seems to be different from the currently approved HCV drugs. We expect that the new inhibitors of HCV NS3 helicase discovered herein could be used as a starting point to design potent inhibitors against HCV.

### Experimental Procedures

### Example 10

Materials.

The oligonucleotides were purchased from Integrated DNA Technologies (Coralville, Iowa). Fluorescein-labeled oligonucleotides were purchased from Operon Technologies (Alameda, Calif.). ATP, acrylamide, MOPS, Tris, EDTA, NaC1, MgCl₂, βME, glycerol, bromophenol blue, IPTG, dextrose, PMSF, kanamycin, chloramphenicol, SDS, and NADH were from Fisher (Fairlawn, N.J.). PKILDH, BSA, PEP, pepstatin A, lysozyme, heparin, and Sephadex G-25 were obtained from Sigma (Selma, Calif.). Ethanol was purchased from Pharmco (Brookfield, Conn.). NZCYM broth and Bacto-agar were from Difco laboratories (Lawrence, Kans.). Poly(dT) was from Amersham Biosciences (Piscataway, N.J.). [γ³²-P]ATP was from PerkinEhner Life Sciences (Boston, Mass.). T4 polynucleotide kinase was purchased from New England Biolabs (Ipswich, Mass.). The chromatographic resins for NS3 helicase purification were from Bio-Rad (Hercules, Calif.).

### Assay to Screen for Inhibition of NS3 Helicase Activity

A fluorescence based quantitative assay that measures helicase activity over time was employed to screen the compounds. The concentration of enzyme and compounds used was 250 nM and 20 μM respectively. Helicase catalyzes the unwinding of a FAM-labeled dsDNA and the resulting increase in fluorescence is plotted (λₑₓ-485 nm; λₑₘ, −528 nm). The slope of the initial part of the plot was used to calculate the percentage helicase activity. The compounds exhibiting a significant decrease in activity from the control experiment were considered as possible inhibitors. The IC₅₀ value for ITBA-3-79, ITBA-3-82 and ITBA-3-85-mediated inhibition of NS3 helicase was determined using the same assay.

Gel-Based Helicase Assay.

Benchtop unwinding assays were performed to analyze the helicase activity of NS3 helicase in the presence of the compounds. The unwinding reaction was initiated by mixing the right (N53, compound, assay buffer) and left (radiolabeled substrate, ATP, assay buffer) reactions, which was then quenched. The ssDNA product formed over time is separated from dsDNA substrate by 20% native PAGE. The ratio of dsDNA substrate to ssDNA product is quantified using ImageQuant and the fraction of substrate unwound at each time-point is plotted as a function of time using Kaleidagraph software.

Analysis of the Protease Activity of NS3 in the Presence of the Compounds.

The protease activity of NS3 was analyzed in the presence of 25 μM ITBA-3-79, ITBA-3-82 or ITBA-3-85 by using 50 nM NS3-4A and 100 nM substrate (Ac-Asp-Glu-Asp-EDANS-Glu-Glu-Abu-L-Lactoyl-Ser-Lys DABCYL-NH2). The emission spectra of EDANS and the absorption spectra of DABCYL overlap making the peptide internally quenched. Cleaving of the substrate by the protease results in an increase in fluorescence that can be measured (λₑₓ−355 nm; λₑₘ, −500 nm). Telapravir (10 μM) was used as a positive control.

Analysis of the ATPase Activity of NS3 in the Presence of the Compounds.

The ATPase activity of NS3 (50 nM) was analyzed in the presence of ITBA-3-79, ITBA-3-82 and ITBA-3-85 using a coupled spectrophotometric assay (Raney and Benkovic 1995). The reaction mixture contained 50 mM MOPS, 10 mM NaCl, 10 mM MgCl2, 5 mM ATP, 4 mM PEP, 21.6 U/mL PK, 33.2 U/mL LDH, 0.9 mM NADH, and 2 mM βME. NS3 (50 nM) was added to this reaction mixture. The change in absorbance at 380 nm was monitored for 1 min following addition of DNA (100 μM poly dT). ATP hydrolysis rates were determined by measuring the conversion of NADH to NAD⁺ at 380 nm (^{G}380 of NADH is 1210 M⁻¹ cm⁻¹) and is then directly correlated to ATP hydrolysis. The oxidation of 1 mol of NADH corresponds to the hydrolysis of 1 mol of ATP.

### Example 11

### Specificity of EndoG Inhibitors to Other Enzymes

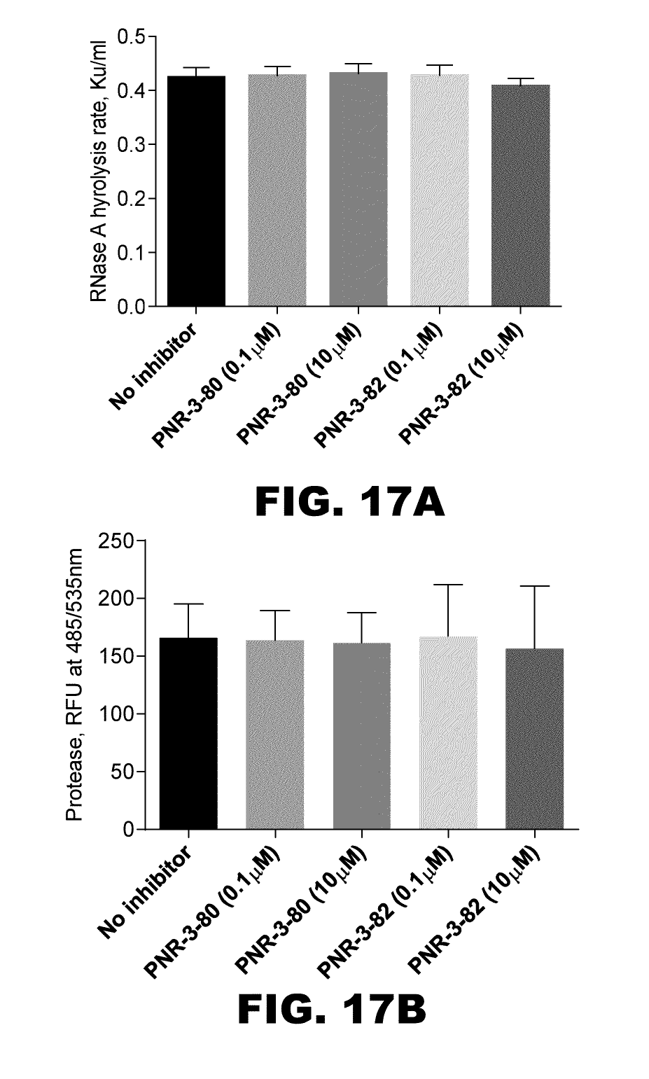

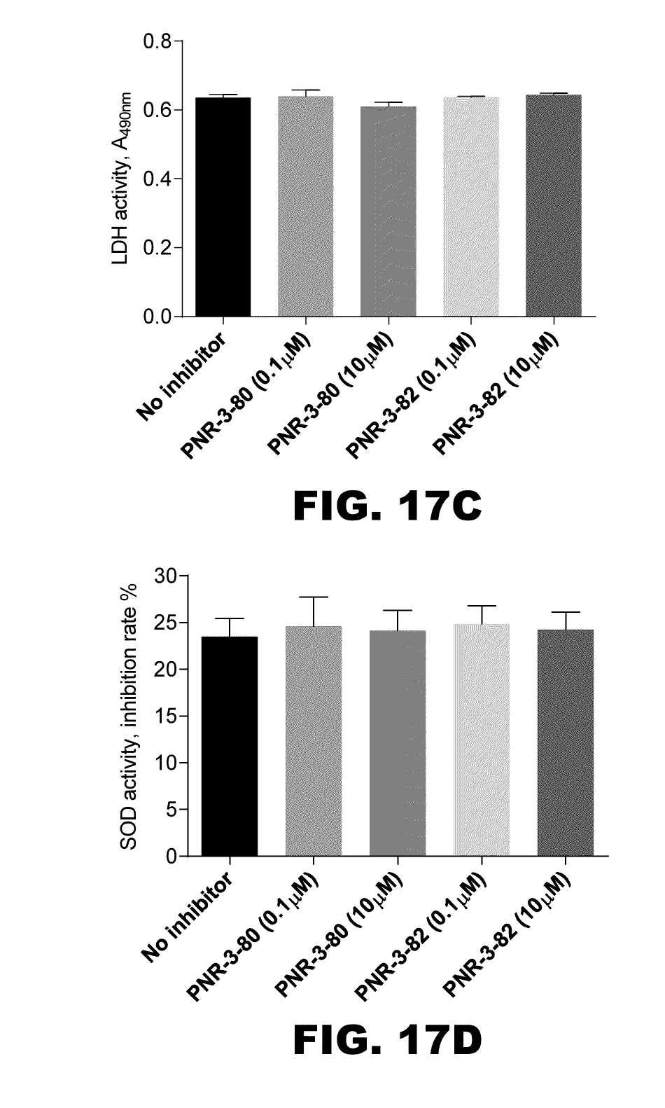

As a follow up to Example 8, the effects of the EndoG inhibitors PNR-3-80 and PNR-3-82, at 0.1 μM and 10 μM each, on the activities of four other enzymes including RNase A, protease, LDH, and SOD in NRK-52E cell extract were examined. NRK-52E cells were grown to ˜80% confluence in 10 mm culture dish. The medium was aspirated and the cells were rinsed with ice cold 1×PBS, pH 7.4. The cells were lysed in 50 mM Tris-HCl, pH 7.4, 150 mM NaCl, 1% Triton X-100 for 10 min on ice and then briefly sonicated. Cell debris was removed by centrifugation at 13,000×g for 10 min at 4° C. The supernatant was collected and stored at −80° C. until use. LDH, Protease, Superoxide dismutase (SOD), and Ribonuclease A (RNase A) activities were measured by using CytoTox 96® Non-Radioactive Cytotoxicity Assay kit (Promega, Madison, Wis.), Protease Fluorescent Detection kit (Sigma-Aldrich, Saint Louis, Mo.), SOD determination kit (Sigma-Aldrich, Saint Louis, Mo.), and Ribonuclease A Detection kit (Sigma-Aldrich, Saint Louis, Mo.), respectively, according to the manufacturer's instructions. The experiment showed that none of the two tested compounds had any inhibiting activity (FIG. 17). Taken together with Example 8, these data suggest that the identified inhibitors are highly specific to EndoG as compared to other tested enzymes, both nucleases and enzymes that are not nucleases.

### Example 12

### Cytoprotection Against Cisplatin-Induced Cell Death

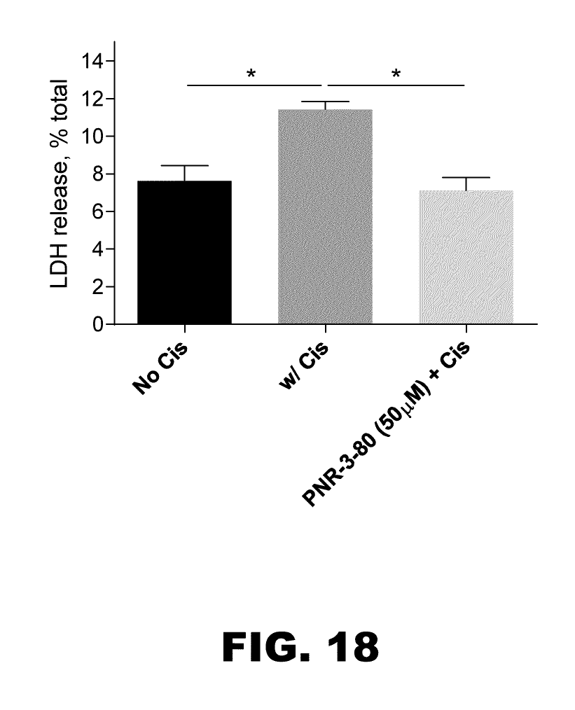

To test cytotoxic activities of the inhibitors, human prostrate carcinoma epithelial 22Rv1 cells, which naturally express EndoG, were exposed to Cisplatin (60 μM) in the presence or absence of PNR-3-80 (50 μM) and cell death was measured by the LDH release assay. In this experiment, PNR-3-80 showed significant and complete inhibition of Cisplatin-induced cell death compared to the control without inhibitor (FIG. 18).

### Example 13

### Cytoprotection Against Docetaxel-Induced Cell Death

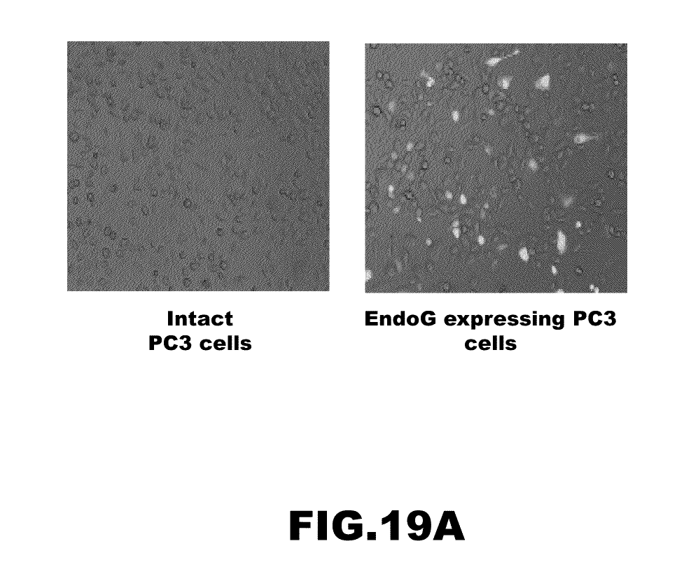

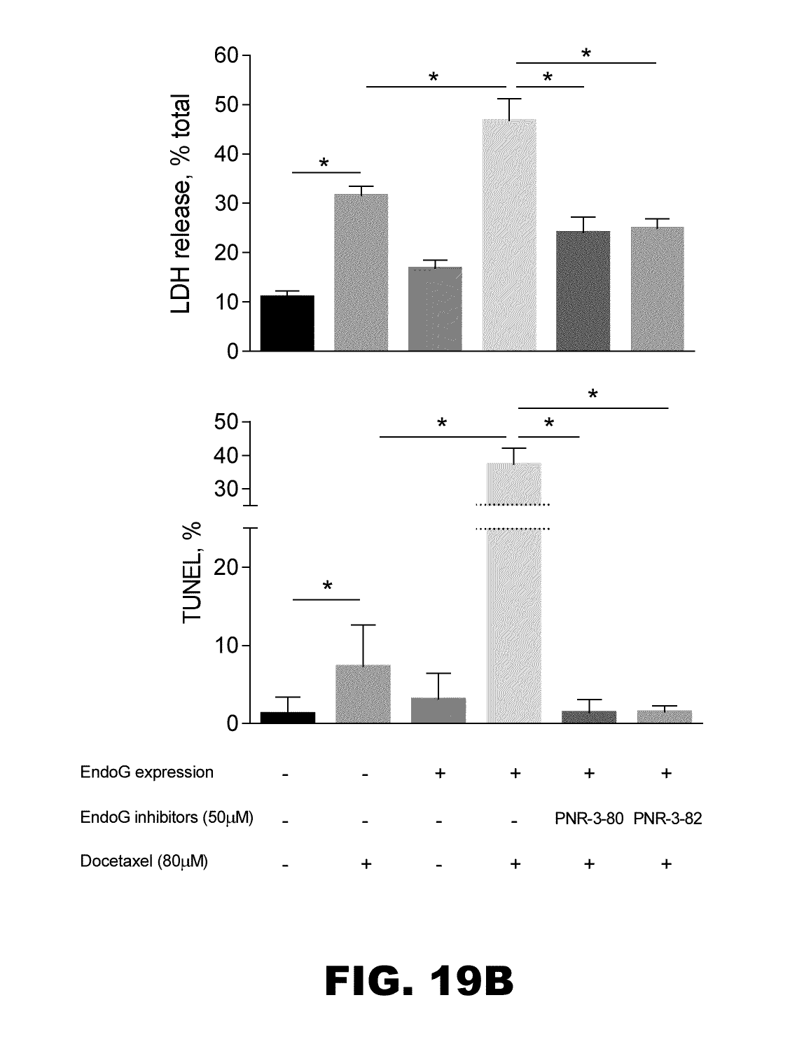

Docetaxel is an anti-cancer drug used to treat prostate cancer. Human invasive prostate cancer PC3 cells are known to be resistant to anticancer drugs. To make these cells sensitive to Docetaxel and to have a model that could be used to prove that the inhibitors act through EndoG, PC3 cells were transfected with EndoG gene bound to cyan fluorescent protein (CFP). The resulting EndoG-expressing PC3 cells were exposed with Docetaxel (80 μM) in the absence or presence of the inhibitors, PNR-3-80 or PNR-3-82 (50 μM each). The cell death was measured by using two methods, LDH release assay and TUNEL assay. The cell death was measured by using two methods, LDH release assay and TUNEL assay. The experiment showed that both inhibitors are cytoprotective and likely to act through EndoG inhibition (FIG. 19).

### Example 14

### Inhibition of EndoG Activity by Compound Homologs

To determine the activity of benzyl, napthyl substituted indole and 2-methyl indole compounds of the same scaffold as the EndoG inhibitors described herein, a number of compounds shown in TABLE 7 were synthesized and tested using the screening method. The result showed that all tested compounds had some inhibiting activity, and some of them are likely to be at least as potent as PNR-3-80 and P-NR-82.

Compounds that inhibit HCV NS3 helicase using a robust and quantitative fluorescence based helicase assay. Three indole thio-barbituric acid (ITBA) derivatives (ITBA-3-79, ITBA-3-82, and ITBA-3-85) were identified as inhibitors of the HCV NS3 helicase. The IC5c. values for ITBA-79, ITBA-82, and ITBA-85-mediated inhibition of NS3 helicase were 21.6±1.9 μM, 21.4±2.4 μM, and 23.5±1.8 μM respectively. The standard helicase assay using gel electrophoresis confirmed the inhibition of NS3 helicase activity by these compounds. These compounds do not block protease activity and their mechanism of inhibition seems to be different from the currently approved HCV drugs. We expect that the new inhibitors of HCV NS3 helicase discovered herein could be used as a starting point to design potent inhibitors against HCV.

TABLE 7
Inhibition of EndoG activity by (% EndoG
activity in the presence of the compound (1μM)

| | | |
|---|---|---|
| PNR-3-80 | 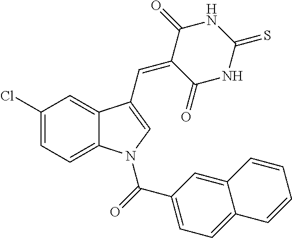 | 36.01 |
| PNR-3-82 | 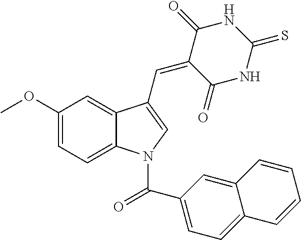 | 21.82 |
| PNR-6-89 | 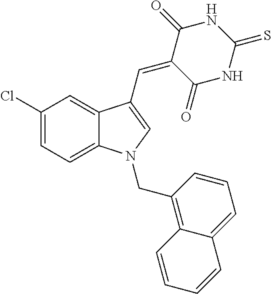 | 3.50 |
| PNR-6-92 | 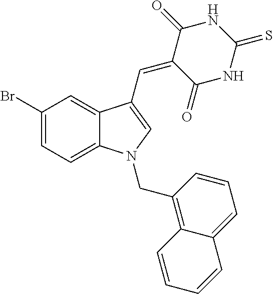 | 8.90 |
| PNR-6-86 | 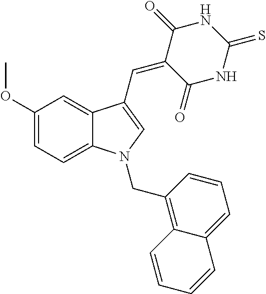 | 9.55 |
| PNR-6-91 | 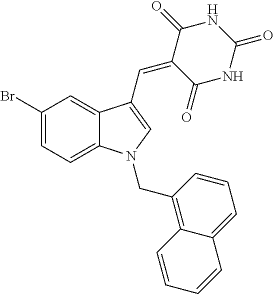 | 12.68 |
| PNR-7-5 | 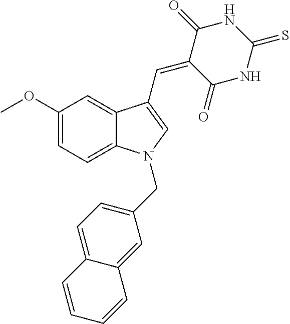 | 13.77 |
| PNR-7-7 | 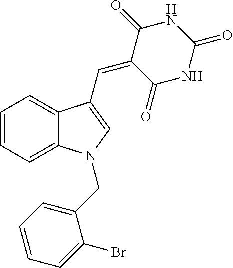 | 14.06 |
| PNR-7-18 | 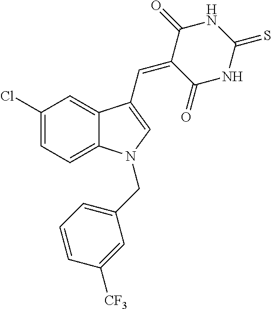 | 14.10 |
| PNR-7-1 | 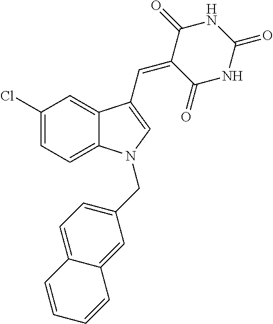 | 14.75 |
| PNR-6-98 | 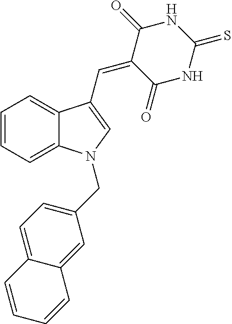 | 15.72 |
| PNR-6-83 | 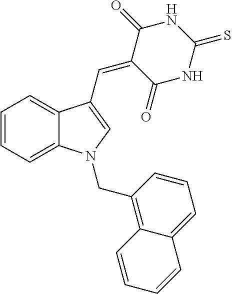 | 15.96 |
| PNR-7-16 | 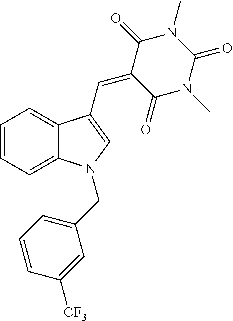 | 17.22 |
| PNR-7-2 | 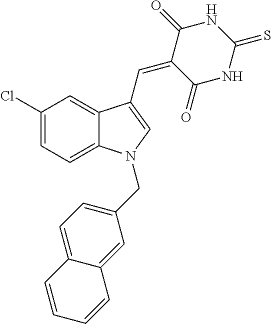 | 17.79 |
| PNR-7-27 | 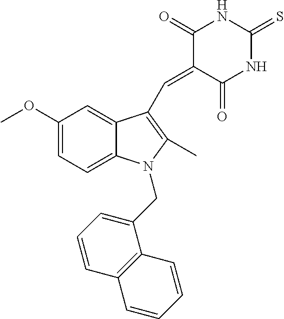 | 19.13 |
| PNR-6-88 | 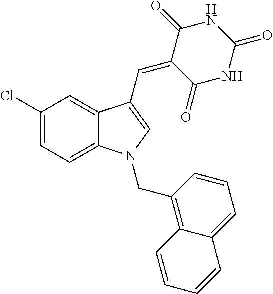 | 22.95 |
| PNR-7-4 | 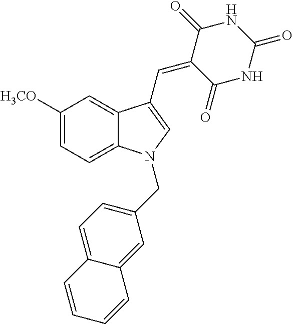 | 24.73 |
| PNR-7-21 | 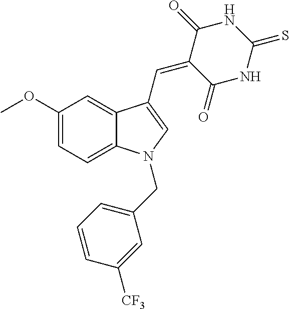 | 28.79 |
| PNR-6-69 | 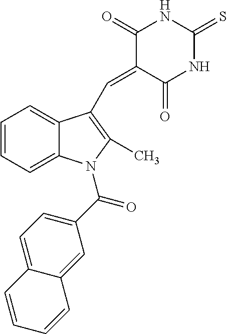 | 31.55 |
| PNR-6-90 | 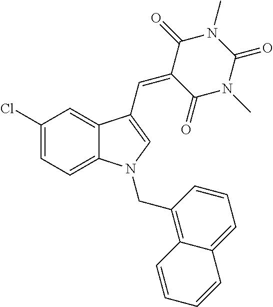 | 35.93 |
| PNR-6-93 | 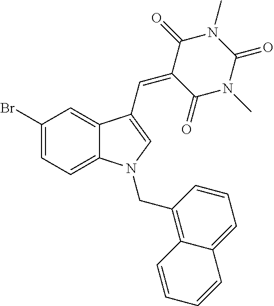 | 45.76 |
| PNR-6-59 | 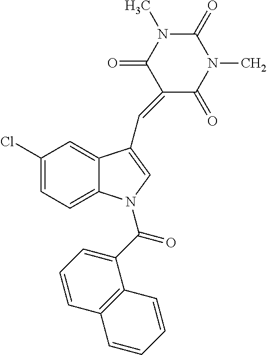 | 47.50 |
| PNR-6-85 | 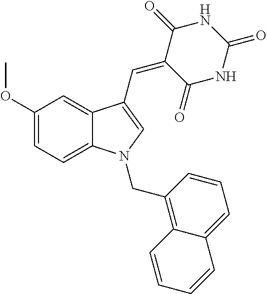 | 52.66 |
| PNR-7-17 | 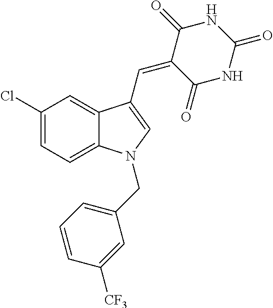 | 57.12 |
| PNR-7-8 | 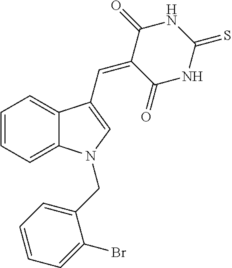 | 58.02 |
| PNR-7-3 | 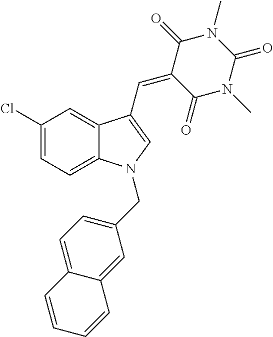 | 59.11 |
| PNR-6-74 | 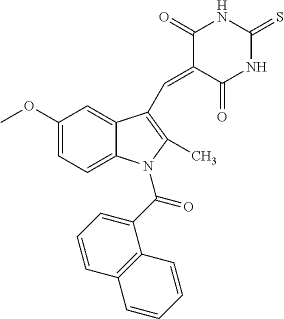 | 63.50 |
| PNR-6-65 | 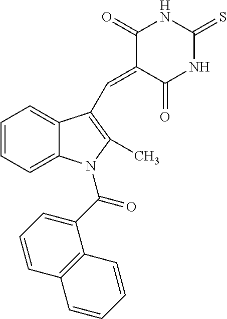 | 64.96 |
| PNR-6-60 | 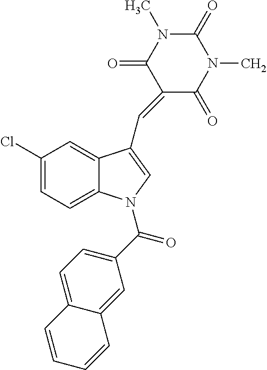 | 67.88 |
| PNR-6-17 | 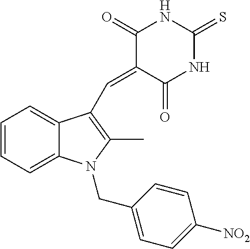 | 69.79 |
| PNR-5-96 | 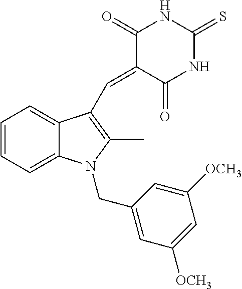 | 71.45 |
| PNR-6-64 | 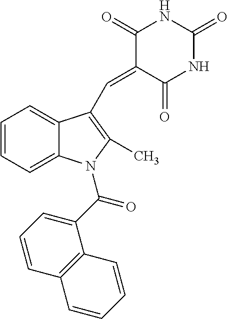 | 71.74 |
| PNR-6-99 | 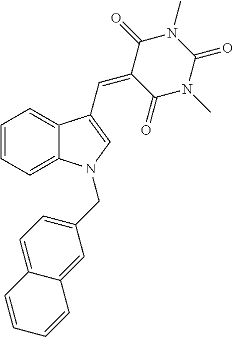 | 71.74 |
| PNR-7-24 | 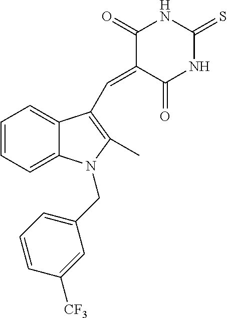 | 73.59 |

1. A compound, the compound comprising a polymerase inhibitor and/or endonuclease inhibitor of Formula (I): 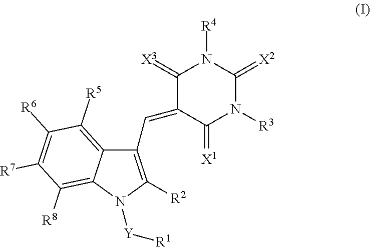
wherein:
X¹, X², and X³ are each independently selected from the group consisting of oxygen, sulfur, and sulfene (R₂C═SO₂);
Y is selected from the group consisting of CH₂, carbonyl, sulfide (R—S(—O)—R), sulfone (R—S(O₂)—R), and sulfoxide (R—S(═O)—R);
R¹ is selected from the group consisting of hydrocarbyl, substituted hydrocarbyl, and cyano;
R², R³, and R⁴ are each independently selected from the group consisting of hydrogen, hydrocarbyl, and substituted hydrocarbyl; and
R⁵, R⁶, R⁷, and R⁸ are each independently selected from the group consisting of hydrogen, hydrocarbyl, substituted hydrocarbyl, trifluoromethyl, halogen, cyano, nitro, amidine, amino, carboxyl, ester, alkylalkylamino, dialkylamino, hydroxyl, alkoxy, and arylalkoxy.

2. The compound of claim 1, the compound comprising a polymerase inhibitor and/or endonuclease inhibitor of Formula (I), wherein:
X¹ and X³ are oxygen;
X² is selected from the group consisting of oxygen, sulfur, and sulfene (R₂C═SO₂);
Y is selected from the group consisting of CH₂, carbonyl, sulfide (R—S(—O)—R), sulfone (R—S(O₂)—R), and sulfoxide (R—S(═O)—R);
R¹ is selected from the group consisting of alkyl, substituted alkyl, alkenyl, substituted alkenyl, alkynyl, substituted alkynyl, aryl, substituted aryl, cyano, and COOCH₃;
R², R³, and R⁴ are each independently selected from the group consisting of hydrogen, hydrocarbyl, and substituted hydrocarbyl; and
R⁵, R⁶, R⁷, and R⁸ are each independently selected from the group consisting of hydrogen, hydrocarbyl, substituted hydrocarbyl, trifluoromethyl, halogen, cyano, nitro, amidine, amino, carboxyl, ester, alkylalkylamino, dialkylamino, hydroxyl, alkoxy, and arylalkoxy.

3. The compound of claim 1, the compound comprising a polymerase inhibitor and/or endonuclease inhibitor of Formula (I), wherein:
X¹ and X³ are oxygen;
X² is selected from the group consisting of oxygen, sulfur, and sulfene (R₂C═SO₂);
Y is selected from the group consisting of CH₂, carbonyl, sulfide (R—S(—O)—R), sulfone (R—S(O₂)—R), and sulfoxide (R—S(═O)—R);
R¹ is selected from the group consisting of alkyl, substituted alkyl, alkenyl, substituted alkenyl, alkynyl, substituted alkynyl, aryl, substituted aryl, cyano, and COOCH₃;
R², R³, and R⁴ are selected from the group consisting of hydrogen, hydrocarbyl, and substituted hydrocarbyl;
R⁵ and Ware hydrogen; and
R⁶ and R⁷ are each independently selected from the group consisting of hydrogen, hydrocarbyl, substituted hydrocarbyl, halogen, cyano, nitro, amidine, amino, carboxyl, ester, alkylalkylamino, dialkylamino, hydroxyl, alkoxy, and arylalkoxy.

4. The compound of claim 1, the compound comprising a polymerase inhibitor and/or endonuclease inhibitor of Formula (I), wherein:
X¹ and X³ are oxygen;
X² is selected from the group consisting of oxygen, sulfur, and sulfene (R₂C═SO₂);
Y is selected from the group consisting of CH₂, carbonyl, sulfide (R—S(—O)—R), sulfone (R—S(O₂)—R), and sulfoxide (R—S(═O)—R);
R¹ is selected from the group consisting of alkyl, substituted alkyl, alkenyl, substituted alkenyl, alkynyl, substituted alkynyl, aryl, substituted aryl, cyano, and COOCH₃;
R² is selected from the group consisting of hydrogen, methyl, phenyl, and substituted phenyl;
R³ and R⁴ are selected from the group consisting of hydrogen, hydrocarbyl, and substituted hydrocarbyl;
R⁵ and R⁸ are hydrogen; and
R⁶ and R⁷ are each independently selected from the group consisting of hydrogen, hydrocarbyl, substituted hydrocarbyl, halogen, cyano, nitro, amidine, amino, carboxyl, ester, alkylalkylamino, dialkylamino, hydroxyl, alkoxy, and arylalkoxy.

5. The compound of claim 1, the compound comprising a polymerase inhibitor and/or endonuclease inhibitor of Formula (I), wherein:
X¹ and X³ are oxygen;
X² is sulfur;
Y is selected from the group consisting of CH₂, carbonyl, sulfide (R—S(—O)—R), sulfone (R—S(O₂)—R), and sulfoxide (R—S(═O)—R);
R¹ is selected from the group consisting of phenyl, substituted phenyl, biphenyl, substituted biphenyl, naphthyl, substituted naphthyl, cyano, and COOCH₃;
R² is selected from the group consisting of hydrogen, methyl, phenyl, and substituted phenyl;
R³ and R⁴ are selected from the group consisting of hydrogen, hydrocarbyl, and substituted hydrocarbyl;
R⁵ and R⁸ are hydrogen; and
R⁶ and R⁷ are each independently selected from the group consisting of hydrogen, hydrocarbyl, substituted hydrocarbyl, trifluoromethyl, halogen, cyano, nitro, amidine, amino, carboxyl, ester, alkylalkylamino, dialkylamino, hydroxyl, alkoxy, and arylalkoxy.

6. The compound of claim 1, the compound comprising a polymerase inhibitor and/or endonuclease inhibitor of Formula (I), wherein:
X¹ and X³ are oxygen;
X² is selected from the group consisting of oxygen, sulfur, and sulfene (R₂C═SO₂);
Y is selected from the group consisting of CH₂, carbonyl, sulfide (R—S(—O)—R), sulfone (R—S(O₂)—R), and sulfoxide (R—S(═O)—R);
R¹ is selected from the group consisting of alkyl, substituted alkyl, alkenyl, substituted alkenyl, alkynyl, substituted alkynyl, aryl, substituted aryl, cyano, and COOCH₃;
R² is selected from the group consisting of hydrogen, methyl, phenyl, and substituted phenyl;
R³, R⁴, R⁵, and R⁸ are hydrogen; and
R⁶ and R⁷ are each independently selected from the group consisting of hydrogen, hydrocarbyl, substituted hydrocarbyl, halogen, cyano, nitro, amidine, amino, carboxyl, ester, alkylalkylamino, dialkylamino, hydroxyl, alkoxy, and arylalkoxy.

7. The compound of claim 1, the compound comprising a polymerase inhibitor and/or endonuclease inhibitor of Formula (I), wherein:
X¹ and X³ are oxygen;
X² is selected from the group consisting of oxygen, sulfur, and sulfene (R₂C═SO₂);
Y is carbonyl;
R¹ is selected from the group consisting of phenyl, substituted phenyl, biphenyl, substituted biphenyl, naphthyl, substituted naphthyl, cyano, and COOCH₃;
R² is selected from the group consisting of hydrogen, methyl, phenyl, and substituted phenyl;
R³ and R⁴ are selected from the group consisting of hydrogen, hydrocarbyl, and substituted hydrocarbyl;
R⁵ and R⁸ are hydrogen; and
R⁶ and R⁷ are each independently selected from the group consisting of hydrogen, hydrocarbyl, substituted hydrocarbyl, trifluoromethyl, halogen, cyano, nitro, amidine, amino, carboxyl, ester, alkylalkylamino, dialkylamino, hydroxyl, alkoxy, and arylalkoxy.

8. The compound of claim 1, the compound comprising a polymerase inhibitor and/or endonuclease inhibitor of Formula (I), wherein:
X¹ and X³ are oxygen;
X² is sulfur;
Y is carbonyl;
R¹ is selected from the group consisting of alkyl, substituted alkyl, alkenyl, substituted alkenyl, alkynyl, substituted alkynyl, aryl, substituted aryl, cyano, and COOCH₃;
R² is selected from the group consisting of hydrogen, methyl, phenyl, and substituted phenyl;
R³, R⁴, R⁵, and R⁸ are hydrogen; and
R⁶ and R⁷ are each independently selected from the group consisting of hydrogen, hydrocarbyl, substituted hydrocarbyl, halogen, cyano, nitro, amidine, amino, carboxyl, ester, alkylalkylamino, dialkylamino, hydroxyl, alkoxy, and arylalkoxy.

9. The compound of claim 1, the compound comprising a polymerase inhibitor and/or endonuclease inhibitor of Formula (IV): 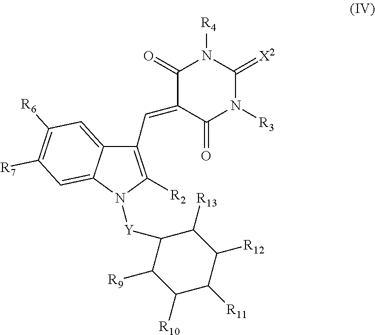
wherein:
X² is selected from the group consisting of oxygen, sulfur, and sulfene (R₂C═SO₂);
Y is selected from the group consisting of CH₂, carbonyl, sulfide (R—S(—O)—R), sulfone (R—S(O₂)—R), and sulfoxide (R—S(═O)—R);
R² is selected from the group consisting of hydrogen, methyl, phenyl, and substituted phenyl;
R³ and R⁴ are selected from the group consisting of hydrogen and methyl;
R⁶, R⁷, R⁹, R¹⁰, R¹¹, R¹², and R¹³ are each independently selected from the group consisting of hydrogen, hydrocarbyl, substituted hydrocarbyl, trifluoromethyl, halogen, cyano, nitro, amidine, amino, carboxyl, ester, alkylalkylamino, dialkylamino, hydroxyl, alkoxy, and arylalkoxy.

10. The compound of claim 9, the compound comprising a polymerase inhibitor and/or endonuclease inhibitor of Formula (IV), wherein:
X² is selected from the group consisting of oxygen, sulfur, and sulfene (R₂C═SO₂);
Y is selected from the group consisting of CH₂, carbonyl, sulfide (R—S(—O)—R), sulfone (R—S(O₂)—R), and sulfoxide (R—S(═O)—R);
R² is selected from the group consisting of hydrogen, methyl, phenyl, and substituted phenyl;
R³ and R⁴ are hydrogen;
R⁶, R⁷, R⁹, R¹⁰, R¹¹, R¹², and R¹³ are each independently selected from the group consisting of hydrogen, hydrocarbyl, substituted hydrocarbyl, trifluoromethyl, halogen, cyano, nitro, amidine, amino, carboxyl, ester, alkylalkylamino, dialkylamino, hydroxyl, alkoxy, and arylalkoxy.

11. The compound of claim 9, the compound comprising a polymerase inhibitor and/or endonuclease inhibitor of Formula (IV), wherein:
X² is selected from the group consisting of oxygen, sulfur, and sulfene (R₂C═SO₂);
Y is selected from the group consisting of CH₂, carbonyl, sulfide (R—S(—O)—R), sulfone (R—S(O₂)—R), and sulfoxide (R—S(═O)—R);
R², R³, and R⁴ are hydrogen;
R⁶, R⁷, R⁹, R¹⁰, R¹¹, R¹², and R¹³ are each independently selected from the group consisting of hydrogen, hydrocarbyl, substituted hydrocarbyl, trifluoromethyl, halogen, cyano, nitro, amidine, amino, carboxyl, ester, alkylalkylamino, dialkylamino, hydroxyl, alkoxy, and arylalkoxy.

12. The compound of claim 9, the compound comprising a polymerase inhibitor and/or endonuclease inhibitor of Formula (IV), wherein:
X² is sulfur;
Y is selected from the group consisting of CH₂, carbonyl, sulfide (R—S(—O)—R), sulfone (R—S(O₂)—R), and sulfoxide (R—S(═O)—R);
R², R³, and R⁴ are hydrogen;
R⁶, R⁷, R⁹, R¹⁰, R¹¹, R¹², and R¹³ are each independently selected from the group consisting of hydrogen, hydrocarbyl, substituted hydrocarbyl, trifluoromethyl, halogen, cyano, nitro, amidine, amino, carboxyl, ester, alkylalkylamino, dialkylamino, hydroxyl, alkoxy, and arylalkoxy.

13. (canceled)

14. The compound of claim 1,
wherein the IC50 value for the compound comprising Formula (I) against an enzyme chosen from polymerase and endonuclease is below 50 μM.

15.-16. (canceled)

17. A method of treating a tumor, the method comprising contacting a tumor cell with a composition comprising a polymerase inhibitor of Formula (I): 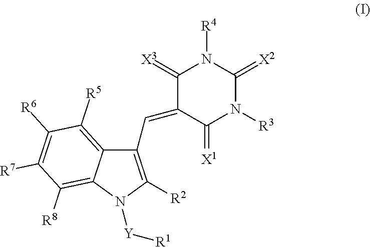
wherein:
X¹, X², and X³ are each independently selected from the group consisting of oxygen, sulfur, and sulfene (R₂C═SO₂);
Y is selected from the group consisting of CH₂, carbonyl, sulfide (R—S(—O)—R), sulfone (R—S(O₂)—R), and sulfoxide (R—S(═O)—R);
R¹ is selected from the group consisting of hydrocarbyl, substituted hydrocarbyl, cyano, and COOCH₃;
R², R³, and R⁴ are each independently selected from the group consisting of hydrogen, hydrocarbyl, and substituted hydrocarbyl; and
R⁵, R⁶, R⁷, and R⁸ are each independently selected from the group consisting of hydrogen, hydrocarbyl, substituted hydrocarbyl, trifluoromethyl, halogen, cyano, nitro, amidine, amino, carboxyl, ester, alkylalkylamino, dialkylamino, hydroxyl, alkoxy, and arylalkoxy.

18. The method of claim 17, wherein the tumor cell is selected from the group consisting of a pancreatic tumor cell, a breast cancer cell, an ovarian cancer cell, a cervical cancer cell, a uterine cancer cell, a prostate cancer cell, a lung cancer cell, a brain cancer cell, and a combination thereof.

19. The method of claim 17, wherein Formula (I) is chosen from ITBA-17 or ITBA-3.

20. (canceled)

21. The method of claim 22, wherein the compound comprising Formula (I) is chosen from PNR-3-80 and PNR-3-82.

22. A method of protecting a cell from death, the method comprising contacting a cell with a composition comprising an endonuclease inhibitor of Formula (I): 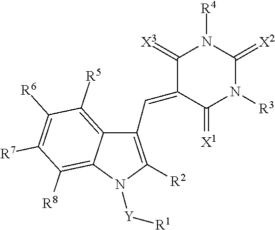
wherein:
X¹, X², and X³ are each independently selected from the group consisting of oxygen, sulfur, and sulfene (R₂C═SO₂);
Y is selected from the group consisting of CH₂, carbonyl, sulfide (R—S(—O)—R), sulfone (R—S(O₂)—R), and sulfoxide (R—S(═O)—R);
R¹ is selected from the group consisting of hydrocarbyl, substituted hydrocarbyl, cyano, and COOCH₃;
R², R³, and R⁴ are each independently selected from the group consisting of hydrogen, hydrocarbyl, and substituted hydrocarbyl; and
R⁵, R⁶, R⁷, and R⁸ are each independently selected from the group consisting of hydrogen, hydrocarbyl, substituted hydrocarbyl, trifluoromethyl, halogen, cyano, nitro, amidine, amino, carboxyl, ester, alkylalkylamino, dialkylamino, hydroxyl, alkoxy, and arylalkoxy.

23. The method of claim 22, wherein the cell expresses endonuclease G.

24. The method of claim 22, wherein cell death is induced by a cell injury selected from the group consisting of chemical poisoning, drug poisoning, radiation, hypoxia, physical injury, and chemotherapeutics.

25.-30. (canceled)
